SEQUENCE LISTING (2 sequences)

SEQ ID NO: 1 - protein, 231 aa, Homo sapiens
MAGKKVLIVYAHQEPKSFNGSLKNVAVDELSRQGCTVTVSDLYAMNLEPRATDKDITGTLSNPEVFNYGVETHEAYKQRSLASDITDEQKKVREADLVIFQFPLYWFSVPAILKGWMDRVLCQGFAFDIPGFYDSGLLQGKLALLSVTTGGTAEMYTKTGVNGDSRYFLWPLQHGTLHFCGFKVLAPQISFAPEIASEEERKGMVAAWSQRLQTIWKEEPIPCTAHWHFGQ

SEQ ID NO: 2 - protein, 274 aa, Homo sapiens
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

(19) United States
(12) Patent Application Publication
(10) Pub. No.: US 2024/0228481 A1
(43) Pub. Date: Jul. 11, 2024

(54) NOVEL SULFONAMIDE SERIES OF QR2 INHIBITORS TO TACKLE OXIDATIVE STRESS AND COGNITIVE DECLINE

(71) Applicant: CARMEL HAIFA UNIVERSITY ECONOMIC CORPORATION LTD., Haifa (IL)

(72) Inventors: Kobi ROSENBLUM, Zikhron Ya'akov (IL); Natheniel GOULD, Haifa (IL); Efrat EDRI, Kiryat Ata (IL)

(21) Appl. No.: 18/284,840

(22) PCT Filed: Mar. 30, 2022
(86) PCT No.: PCT/IL2022/050341

§ 371 Date: Sep. 28, 2023

Related U.S. Application Data
(60) Provisional application No. 63/279,486, filed on Nov. 15, 2021.
(60) Provisional application No. 63/167,721, filed on Mar. 30, 2021.

(51) Int. Cl. C07D 471/04; A61K 31/437; A61K 31/4545; A61K 31/496; A61P 25/28

(52) U.S. Cl. CPC C07D 471/04; A61K 31/437; A61K 31/4545; A61K 31/496; A61P 25/28

(57) ABSTRACT

The present invention provides an aromatic sulfonamide-based compound, a pharmaceutical composition comprising same, and methods of using same, such as for inhibiting quinone reductase 2 (QR2) or treating a QR2-related disease or disorder.

### CROSS-REFERENCE TO RELATED APPLICATIONS

This application claims the benefit of priority of U.S. Provisional Patent Application Nos. 63/167,721, titled “NOVEL SULFAMIDE SERIES OF QR2 INHIBITORS TO TACKLE OXIDATIVE STRESS AND COGNITIVE DECLINE”, filed Mar. 30, 2021, and 63/279,486, titled “NOVEL SULFAMIDE SERIES OF QR2 INHIBITORS AND USES THEREOF”, filed Nov. 15, 2021. The contents of the above applications are all incorporated by reference as if fully set forth herein in their entirety.

### FIELD OF INVENTION

The present invention relates to quinone reductase 2 (QR2) inhibitors, methods for producing same, and use thereof, such as for treating age related oxidative stress and cognitive decline, in a subject in need thereof.

### BACKGROUND

Accumulating metabolic dysfunction in the ageing brain creates chronic stressors, which result in a wide spectrum of pathologies and eventual memory deterioration. This is particularly noted in cases where these damaging and inflammatory developments end up defining neurodegenerative diseases, which lead to dementia.

The QR2 pathway was recently highlighted as an element in the process of novel memory formation, which is gradually lost with age and dementia. The QR2 enzyme was found to act downstream of dopamine (DA) and/or acetylcholine (ACh) following a novel experience or event, as a removable memory constraint within inhibitory interneurons. Although much remains unknown regarding QR2 in the brain, how it functions, or whether it is tied to the better studied molecular consolidation processes known to underpin memory formation, the pathway has been broadly described. Namely, DA/ACh drive an increase in microRNA (miR)-182, which in turn reduces QR2 expression, thus removing QR2 mediated reactive oxygen species (ROS) generation and reducing oxidation levels, resulting in decreased inhibitory interneuron activity, and a shift in excitation/inhibition within defined brain areas, 3 h following a learning experience. The result of this QR2 pathway activation is enhanced memory formation for a ‘first time’. Effectively, QR2 removal creates the distinct memory formed for novel events, allowing such important and salient experiences to stand out from continuously perceived familiar or unimportant information. Critically, elements upstream to QR2 within this pathway, which reduce QR2 expression, are lost with age, including DA, ACh and miR-182. The result is chronically elevated levels of QR2, which are most distinct in neurodegenerative diseases such as Alzheimer's (AD) and Parkinson's (PD) diseases. This is injurious twofold—first, the removal of QR2 from interneurons is critical for novel memory formation and this process is lost; and second, aberrantly elevated levels of QR2 cause oxidative stress. Thus, QR2 pathway dysregulation contributes both to the anterograde amnesia and chronic metabolic stress that define age related cognitive decline and dementia.

Conversely, in the scopolamine model of dementia, QR2 inhibition reverses memory deficits in rats completely, pointing to its centrally important role downstream of neuromodulation which is depleted with age and neurodegenerative diseases. Hence, in the case of AD, by acting downstream of ACh esterase (AChE) inhibitors that are dependent on rapidly diminishing cholinergic inputs, direct QR2 inhibition may provide a longer lasting and disease modifying route for AD treatment, replacing the dependence on ACh. Due to such promise, both in AD and others, efforts to develop QR2 inhibitors for various therapeutic approaches have been consistently made over the past two decades. However, despite the fact that QR2 has only one other closely related enzyme, NQO1, making it a simpler target to inhibit specifically, the existing inhibitors for QR2 remain either non-specific, insoluble/non-bioavailable or toxic, which raises many difficulties in studying QR2 and has so far prevented any druggable target for potential therapeutic use from being developed.

Therefore, there is a great need for specific, bioavailable, non-toxic, and effective QR2 inhibitors, and methods of using same.

### SUMMARY

According to a first aspect, there is provided a pharmaceutical composition for use in the treatment or prevention of a quinone reductase 2 (QR2)-related disease or disorder in a subject in need thereof, comprising a compound represented by or comprises Formula 1:

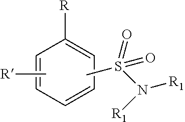

wherein:
   [symbol] represents a single or a double bond;
R′ is any one of:

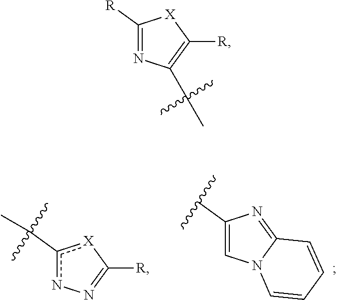

each R independently represents hydrogen, or a substituent comprising halogen, —NO₂, —CN, —OH, —CONH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof;
each X independently comprises C, CH, S, O, N or NH;
each R₁ independently comprises optionally substituted C₁-C₆ alkyl, hydroxy(C₁-C₆ alkyl), C₁-C₆ haloalkyl, optionally substituted C₃-C₈ cycloalkyl, optionally substituted C₁-C₆ alkyl-cycloalkyl, optionally substituted C₁-C₆ alkyl-aryl, optionally substituted C₁-C₆ alkyl-heteroaryl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or both R1 are interconnected, so as to form a cyclic ring; an acceptable salt thereof, or any combination thereof, or any salt thereof.

According to another aspect, there is provided a method for preventing or treating a QR2-related disease or disorder in a subject in need thereof, the method comprising administering to the subject a therapeutically effective amount of the herein disclosed pharmaceutical composition, thereby preventing or treating a QR2-related disease or disorder in the subject.

According to another aspect, there is provided a method for inhibiting or reducing activity of QR2 in a cell, the method comprising contacting the cell with an effective amount of the compound of the invention, thereby inhibiting or reducing activity of QR2 in the cell.

According to some embodiments, the pharmaceutical composition further comprises a pharmaceutically acceptable carrier.

In some embodiments, the compound is represented by or comprises Formula 2:

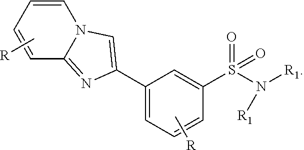

In some embodiments, each R independently represents hydrogen, or a substituent comprising halogen, —NO₂, —CN, —OH, —CONH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, or a combination thereof.

In some embodiments, each R1 independently represents optionally substituted C₁-C₆ alkyl, optionally substituted C₁-C₆ alkyl, optionally substituted C₁-C₆ alkyl-cycloalkyl, optionally substituted C₁-C₆ alkyl-heterocyclyl, optionally substituted C₁-C₆ alkyl-aryl, optionally substituted C₁-C₆ alkyl-heteroaryl, optionally substituted heteroaryl, optionally substituted aryl, optionally substituted cycloalkyl, optionally substituted heterocyclyl, optionally substituted bicyclic heteroaryl or a combination thereof.

In some embodiments, the compound is represented by or comprises Formula 3:

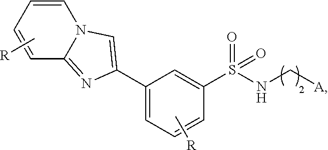

wherein A comprises optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof.

In some embodiments, the compound is represented by or comprises Formula 4:

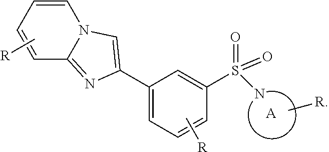

In some embodiments, A is selected from the group consisting of: pyrrolidine, oxirane, tetrahydrofuran, aziridine, pyran, dioxane, thiolane, oxathiolane, piperidine, morpholine, and any combination thereof.

In some embodiments, the compound is represented by or comprises Formula 5:

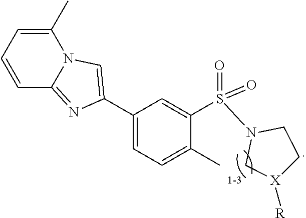

In some embodiments, X is selected from C, CH, N, and NH.

In some embodiments, R comprises any one of —C(═O)R₃, —C(═N)R₃, —C(═O)OR₃, —C(═O)NR₃, —C(═S)OR₃, —C(═S)NR₃; wherein R₃ represents hydrogen or is selected from the group consisting of: optionally substituted C₁-C₆ alkyl, optionally substituted C₃-C₁₀ cycloalkyl, optionally substituted C₃-C₁₀ heterocyclyl, optionally substituted heteroaryl, optionally substituted aryl, and any combination thereof.

In some embodiments, R is H or C(═O)R₃, and wherein R₃ represents hydrogen, or optionally substituted heteroaryl.

In some embodiments, the compound comprises any one of:

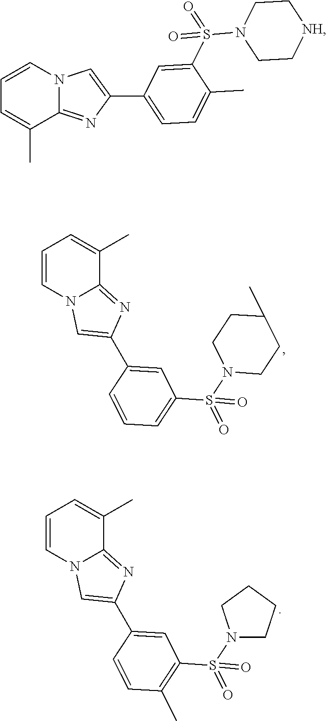 [SMILES: Cc1ccc(-c2cn3cccc(C)c3n2)cc1S(=O)(=O)N1CCNCC1 ; Cc1cccn2cc(-c3cccc(S(=O)(=O)N4CCC(C)CC4)c3)nc12 ; Cc1ccc(-c2cn3cccc(C)c3n2)cc1S(=O)(=O)N1CCCC1]

In some embodiments, the cell is a nerve cell, a glia cell, or both.

In some embodiments, the QR2-related disease or disorder is selected from the group consisting of: a neurodegenerative disease or disorder, a cell-proliferative disease or disorder, malaria, drug direct or indirect toxicity, NAD-related metabolic toxicity, epilepsy, ischemia, depression and/or anxiety, restenosis, glaucoma, immune-response related disease, and any combination thereof.

In some embodiments, the neurodegenerative disease or disorder is selected from the group comprising: Alzheimer's disease, dementia, Parkinson's disease, Huntington's disease, Down syndrome, amyotrophic lateral sclerosis (ALS), prion disease, and any combination thereof.

In some embodiments, preventing or treating comprises inhibiting QR2 function or activity in a cell of the subject.

In some embodiments, administering comprises: oral administration, topical administration, nasal administration, sublingual administration, buccal administration, a systemic administration, or any combination thereof.

In some embodiments, the method further comprises diagnosing the QR2-related disease or disorder in the subject.

In some embodiments, a subject diagnosed with QR2-related disease or disorder is characterized by increased QR2 function or activity compared to a control subject.

In some embodiments, diagnosing comprises determining the function or activity of QR2 in the subject or in a sample derived therefrom.

In some embodiments, preventing or treating comprises: reducing the amount, or level of reactive oxygen species (ROS), modulating autophagy, reducing or inhibiting inflammation, or any combination thereof, in the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1C include a non-limiting flow chart, a graph, molecular structures, and a table, showing that high throughput screen (HTS)-identified sulfonamide compounds further developed by structure-activity-relationship (SAR) medicinal chemistry, provided novel, highly potent, selective, and soluble QR2 inhibitors. (1A) Procedural overview of compound screening and inhibitor development. (1B) A QR2 activity assay based HTS against approximately 200,000 compounds was carried out. The screen includes a fluorescence assay followed by a dose dependent hit confirmation using both a fluorescence and an absorbance assay, which enabled both an orthogonal hit validation, as well as removal of possible fluorescent compounds. This was followed by a selectivity assay that identified the ability of the molecules to specifically inhibit QR2, and not the closely related NQO1 (also known as QR1; inlay). Blue circles—inhibitor control; green circles—neutral control; black circles—compounds; Red circles—sulfonamide series; Red dashed line—30% inhibition threshold. (1C) SAR evaluation of the identified sulfonamide series (top, black) showed distinct QR2 inhibition properties depending on heterocycle (left, blue) and amine (right, green) groups used. By adjusting groups, QR2 specificity and inhibition potency is achieved.

FIGS. 2A-2B include molecular structures, micrographs and a graph showing that novel inhibitors directly bind to QR2 in vitro. (2A) QR2 thermal aggregation curves in HEK293T cells showing increased levels of solubilized receptor at elevated temperatures in the presence of different inhibitors (5 μM concentration) versus vehicle. (2B) Amount of stabilized soluble QR2 in the presence of increasing concentrations of the different compounds (western blot data for QR2 as well as corresponding SOD1 levels). Isothermal dose-response fingerprints of QR2 stabilization by the different compounds tested (at 73° C.). Data are shown as mean±SEM.

FIGS. 3A-3D include illustration of the crystal structure of YB-537 bound to QR2. (3A) Ribbon representation of the human QR2 (hQR2) homodimer (monomer A—red; and monomer B—blue) with stick representation of flavin adenine dinucleotide (FAD; yellow) and YB-537 (green). (3B) Critical interactions with residues in hQR2 are absent in NQO1. These include I128, F131 and N161 (the latter of which forms an important hydrogen bond with YB-537) that are replaced with Y128, M131 and H161, respectively, in NQO1. (3C) ConSurf analysis for hQR2 (surface presentation) and its FAD and YB-537 binding site. The conserved amino acids are shown in maroon and the less conserved in turquoise. While most of the amino acids interacting with the FAD prosthetic group are conserved across hNQO1 and hQR2, the amino acids interacting with YB-537 are not. (3D) Ribbon representation of hNQO1 (monomer A—red; monomer B—blue; FAD-yellow), including the 43 amino acid residue C-terminus (white) which is absent in hQR2, hindering YB-537 (green) access to the catalytic site.

FIGS. 4A-4C include molecular structures, and graphs showing that QR2 inhibitors are non-toxic. (4A) YB-808 shows a median lethal dose (LD₅₀) of 59.7 μM when assessed in THLE cells with CellTiter Glo (middle panel) and displays no toxicity when using XTT assay on HEK293T (right panel. Kruskal-Wallis test, p=0.0180; Dunn's multiple comparisons test, Vehicle vs. YB-808 20 nM, P>0.9999; Vehicle vs. YB-808 200 nM, P>0.9999; Vehicle vs. YB-808 2 μM, p=0.3721; Vehicle vs. YB-808 20 μM, p=0.1232). (4B) YB-800 shows a LD50 of 78.4 μM when assessed in THLE cells with CellTiter Glo (middle panel) and displays no toxicity when using XTT assay on HEK293T (right panel. Kruskal-Wallis test, p=0.0044; Dunn's multiple comparisons test, Vehicle vs. YB-800 20 nM, p=0.0905; Vehicle vs. YB-800 200 nM, p=0.3277; Vehicle vs. YB-800 2 μM, p=0.3277; Vehicle vs. YB-800 20 μM, P>0.9999). (4C) YB-537 shows a LD50 of >100 μM when assessed in THLE cells with CellTiter Glo (middle panel) and displays no toxicity when using XTT assay on HEK293T (right panel. Kruskal-Wallis test, p=0.5637). Data presented as mean±Standard error of the mean (SEM).

FIGS. 5A-5H include illustrations, and graphs showing that novel QR2 inhibitors enhance cortical and hippocampal memory in mice and rats. (5A) Rats were trained to drink from pipettes and were then given 20 μM YB-808 or vehicle prior to being given a novel taste (0.3% NaCl), for which their memory was tested via choice test against water, two days later. (5B) Cannula placement in the rat anterior insular cortex (aIC) was validated. (5C) Rats that received 20 μM YB-808 to the aIC drank significantly more NaCl than the vehicle control group (Vehicle 56.410±4.905%; YB-808 68.67±3.575%; Unpaired t-test, t=2.037 df=33, p=0.0498). (5D) After being administered 5 μM YB-537 or vehicle to Cornu ammonis 1 (CA1), mice underwent delay fear conditioning (DFC), consisting of a 2 min exploration period followed by three bouts of 20 s tone and 2 s footshock upon tone termination, with a 1 min interval between bouts, and prior to removal from the chamber. (5E) Cannula placement in the mouse CA1 formation was validated. (5F) Baseline freezing in mice receiving YB-537 was the same as vehicle controls (Vehicle 1.589±0.390%; YB-537 3.595±1.244%; Mann Whitney U test, p=0.1807). (5G) Mice receiving YB-537 freeze significantly more than vehicle control in response to the context (Vehicle 23.990±3.078%; YB-537 44.740±5.484%; Unpaired t-test, t=3.432 df=11, p=0.0056). (5H) Mice receiving YB-537 showed no difference in freezing compared to vehicle control in response to the cue (Vehicle 21.690±2.453%; YB-537 31.480±7.058%; Unpaired t test, t=1.395 df=11, p=0.1904). Data presented as mean±SEM; *P<0.05; **P<0.01.

FIGS. 6A-6D include vertical bar graphs showing that QR2 inhibition reduces reactive oxygen species (ROS) and rapamycin mediated autophagy. (6A) ROS insult in HEK293 cells is reduced in a dose dependent manner with YB-800, as measured by dichlorodihydrofluorescein diacetate (DCFDA). (6B) Genetically encoded redox sensor roGFP shows QR2 inhibition mediated reduction in cytoplasmic oxidation levels, 2-3 h following ROS insult. (6C) Genetically encoded redox sensor roGFP shows QR2 inhibition mediated reduction in mitochondrial oxidation levels, 24 h following ROS insult. (6D) Autophagy is reduced by QR2 inhibition, 24 h following injurious induction of autophagic flux.

FIGS. 7A-7E include illustrations, micrograph and vertical bar graphs showing that QR2 inhibition reduces inflammation. (7A) Chronic inhibition of QR2 in the brain increases CD73 expression levels. (7B) Copied chronic inhibition to cell lines, to enable inflammation studies. (7C) Chronic inhibition of QR2 in HCT cells increases CD73 expression levels similarly to that seen in brain. (7D) IκB levels tend to increase in RAW cells following QR2 inhibition with LPS induction. (7E) phosphorylated NfκB (p-NfκB) levels tend to decrease in RAW cells following QR2 inhibition with LPS induction.

FIGS. 8A-8C include vertical bar graphs showing QR2 inhibition reduces QR2 activity and ROS following induction of oxidative stress in HEK293 cells. (8A) QR2 activity is seen and is inhibited within HEK293 cells, amongst others, as measured by the rate of fluorescent co-factor oxidation. (8B) HEK293 cells seeded at 25μ 10³ cells/well in 96-well plates were treated with 2 mM H₂O₂ with or without increasing doses or QR2 inhibitors for 3 h. Following incubation, the cells were stained with DCFDA for 45 min, and ROS levels were measured using a fluorimeter with ex/em 485/535 nm. N=3. (8C) ROS was measured in HCT cells, and similarly to HEK293, is reduced following QR2 inhibition. The same HCT cells with CRISPR-Cas9 mediated QR2 knock-out show reduced ROS signal compared to their isogenic controls, and QR2 inhibitors do not cause any further reduction in ROS in those cells that do not have QR2 expression. Data presented as mean±SEM. *p<0.05, ***p<0.0005.

FIG. 9 includes a vertical bar graphs showing that autophagy is down-regulated following QR2 inhibitor treatment in HEK293 cells. Rapamycin (RM; 0.5 μM; autophagy inducer) and compound-treated or untreated HEK293 cells were seeded at 10⁵ cells/well in 24-well pated for 16 hours. After incubation, the Autophagy Detection Kit (Abcam ab 139484) was used to measure autophagic vacuoles in living cells, according to the manufacturer's instructions. Samples were analyzed using the green channel of a flow cytometer (488 nm laser source). N=6; data presented as mean±SEM. *p<0.05.

FIG. 10 includes an illustration of a non-limiting schematic representation of an in vitro blood brain barrier (BBB) model.

FIGS. 11A-11F include graphs showing that QR2 inhibitors bind target in vitro and reproduce QR2 CRISPRi results in isogenic controls. (11A) Isothermal dose-response of QR2 stabilization by ligand-target binding of different inhibitors tested (at 73° C.) using CETSA (EC50; YB-808=13 nM; YB-800=34 nM; YB-537=129 nM). (11B) Cell viability measured with Cell-Titre-Glo assays in response to 72 h incubations with increasing doses of three different inhibitors, using THLE2 cells (LD50; YB-808=59.704 μM; YB-800=78.401 μM; YB-537>100 μM). (11C) Repeat validation of toxicity assays, using XTT in HEK293 cells at physiologically relevant concentrations of three inhibitors, shows no toxicity. (11D) A 3 h incubation with 20 μM YB-800 reduces baseline ROS levels in HCT116 (Vehicle-WT, 1.84e-017±2.554e-017 AU, n=5; YB-800-WT −0.098±0.038 AU, n=5; unpaired t test, t=2.557 df=8, p=0.0338) while no change is seen in HCT116 QR2Δ cells following the treatment (Vehicle-QR2Δ −1.13e-017±3.025e-017 AU, n=5; YB-800-QR2Δ −0.009±0.049 AU, n=5; unpaired t test, t=0.2001 df=8, p=0.8464). (11E) QR2 expression is unchanged in HCT116 cells incubated for 4 d with 2 μM YB-800 (HCT116-Vehicle 1±0.083, n=6, n=6; HCT116-NG800 1.182±0.137, n=6; unpaired t test, t=1.129 df=10, p=0.2851). (11F) CD73 expression is significantly increased in HCT116 cells following 4 d incubation with 2 μM YB-800 (HCT116-Vehicle 1.000±0.039, n=6; HCT116-YB-800 1.478±0.082, n=6; Mann-Whitney test, p=0.0022). Data are shown as mean±SEM; *p<0.05; **p<0.01.

FIGS. 12A-12H include graphs and illustrations showing that novel QR2 inhibitors enhance cortical and hippocampal learning in mice and rats. (12A) Rats were trained to drink from pipettes and were then given a novel taste (0.3% NaCl), for which their memory was tested via choice test against water, two days later. (12B) Rats that were microinjected with 20 μM YB-808 to the aIC drank significantly more NaCl than the vehicle control group (Vehicle 56.410±4.905%; YB-808 68.67±3.575%; unpaired t test, t=2.037 df=33, p=0.0498). (12C) Cannula placement in the rat aIC. (12D) After being administered 5 μM YB-537 or vehicle to CA1, mice underwent delayed fear conditioning (DFC), consisting of a 2 min exploration period followed by three bouts of 20 s tone and 2 s foot shock upon tone termination, with a 1 min interval between bouts, and prior to removal from the chamber. (12E) Baseline freezing in mice receiving 5 μM YB-537 was the same as vehicle controls (Vehicle 1.589±0.390%; YB-537 3.595±1.244%; Mann Whitney test, p=0.1807). (12F) Mice that were microinjected with 5 μM YB-537 freeze significantly more than vehicle control in response to the context (Vehicle 23.990±3.078%; YB-537 44.740±5.484%; unpaired t test, t=3.432 df=11, p=0.0056). (12G) Mice that were microinjected with 5 μM YB-537 show no difference in freezing compared to vehicle control in response to the cue (Vehicle 21.690±2.453%; YB-537 31.480±7.058%; unpaired t test, t=1.395 df=11, p=0.1904). (12H) Mice that were microinjected with 5 μM YB-537 to CA1 once a day, over 5 days show a significant increase in CD73 (Vehicle 1.03±0.1478, n=6; YB-537 1.518±0.1556, n=6; unpaired t test, t=2.278 df=10, p=0.0459).

FIGS. 13A-13G include graphs and micrographs showing that QR2 CRISPRi in a human cell line induces functional proteomic changes antagonistic to that of alzheimer's disease patients cortex. (13A) Two independent QR2Δ HCT cell lines (C3 and C5) show similar patterns of changes in protein expression compared to control cell lines (NS) with significant correlation (Pearson r=0.83, P<0.0001). (13B) Overlap between differentially expressed proteins from current study and from DLPFC tissues of AD patients compared to controls, reported in Johnson et al., (2020). Left—Venn diagram presenting numbers of overlapping proteins. Middle—Significance of enrichment of four functional categories representing contrasting effects of QR2Δ and AD, based on ENRICHR analysis of the overlapping subsets marked by squares. Enrichment was considered significant for FDR adjusted P value <0.05. BioPlanet, KEGG and Gene Ontology databases were utilized. Right—z-score and enrichment significance for the same four functional categories within each of the QR2Δ and AD sets separately. Criteria for significance of enrichment were as above. ** FDR adjusted P value <0.01; *** FDR adjusted P value <0.001. (13C) CRISPRi of QR2 in HCT116 cells significantly reduces baseline ROS levels compared to isogenic, QR2 expressing controls (HCT116, 1.840e-017±2.554e-017 AU, n=5; QR2Δ HCT116, −0.322±0.091 AU, n=5; unpaired t test, t=3.523 df=8, p=0.0078). (13D) Immunoblots of QR2 CRISPRi confirm and validate unlabeled LC-MS proteomic analysis (HCT116, 1±0.222, n=6; QR2Δ HCT116, 0.070±0.015, n=6; unpaired t test, t=4.169 df=10, p=0.0019). (13E) Immunoblot validation of LC-MS confirms QR2 CRISPRi significantly increases NDUFA9 levels (HCT116, 1±0.2406, n=6; QR2Δ HCT116, 2.295±0.278, n=6; unpaired t test, t=3.52 df=10, p=0.0055). (13F) Immunoblot validation of LC-MS confirms QR2 CRISPRi significantly increases CD73 levels (HCT116, 1±0.137, n=6; QR2Δ HCT116, 1.979±0.172, n=6; unpaired t test, t=4.448 df=10, p=0.0012). (13G) Immunoblot images of QR2, NDUFA9, CD73 and Tubulin from QR2Δ HCT116 cells and isogenic controls. Unless stated otherwise, data are shown as mean±SEM; **p<0.01.

FIGS. 14A-14B include illustration of a non-limiting proteome of QR2Δ HCT 116 cell lines opposingly overlaps key pathways in Alzheimer's disease. STRING association network for 258 higher expressed (14A) and 171 lower expressed (14B) proteins between QR2Δ and control (Confidence ≥0.8). Proteins used later for verification are marked in boldface.

FIG. 15 includes a sequence alignment analysis showing comparison of QR2 and QR1 amino-acid sequences and structural motifs. Secondary structure elements of QR2 are labeled above- and of QR1 below the corresponding sequence; α and η-helices are spirals and β-strands are arrows. The residues conserved in both proteins are in red blocks. The letter ‘T’ stands for turn. The sequence alignment was performed using MultAlin. The figure was created using ESPript.

FIGS. 16A-16B include tables showing disclosing pharmacokinetics of YB-537 Oral and intravenous administration. (16A) YB-537 has 82% bioavailability when administered p.o. 50 mg/kg in male mice, with a clearance rate half-life of 60.2 min. (16B) YB-537 arrives at the male mouse brain, peaking 1 h following p.o. administration (203 ng/g), and showing a clearance half-life of 63.5 min.

FIGS. 17A-17J include vertical bar graphs showing that Causes no Changes in General Mouse Physiology or Behavior but Improves Nesting behavior Over Time. (17A) Weight of male and female, 8-9 months old 5×FAD mice over the course of 25 days while consuming YB-537 or water (average weights—Females Vehicle 22.61±0.167 g; Females YB-537 22.76±0.192 g; Males Vehicle 31.72±0.256 g; Males YB-537 32.56±0.279 g). (17B) Drinking rate of water or YB-537 in the cages of the 5×FAD mice. Rate is calculated using the total weight of the mice in the cage, and volume drank over time, per cage (average drinking rate—Females Vehicle 0.219±0.24 ml/g/day; Females YB-537 0.170±0.011 ml/g/day; Males Vehicle 0.204±0.035 ml/g/day; Males YB-537 0.170±0.035 ml/g/day). (17C) No change is seen in the average velocity of 5×FAD mice, regardless of sex (both sexes—Vehicle 10.25±0.442 cm/s, n=17; YB-537 10.9±0.800 cm/s, n=16; Mann-Whitney test, p=0.8173; Males—Vehicle 9.88±0.552 cm/s, n=9; YB-537 10.01±0.611 cm/s, n=9; unpaired t test, t=0.1609 df=16, p=0.8742; Females—Vehicle 10.67±0.713 cm/s, n=8; YB-537 12.03±1.625 cm/s, n=7; Mann-Whitney test, p=0.7789). (17D) No change is seen in the total distance traveled by 5×FAD mice, regardless of sex (both sexes—Vehicle 3051±129.8 cm, n=17; YB-537 3244±237.9 cm, n=16; Mann-Whitney test, p=0.8173; Males—Vehicle 2944±163 cm, n=9; YB-537 2983±179.3 cm, n=9; unpaired t test, t=0.1572 df=16, p=0.8770; Females—Vehicle 3170±209.5 cm, n=8; YB-537 3581±484.5 cm, n=7; Mann-Whitney test, p=0.7789). (17E) No change is seen in meandering of 5×FAD mice, regardless of sex (both sexes—Vehicle 708.9±42.28 deg/cm, n=17; YB-537 733.4±42.8 deg/cm, n=16; Mann-Whitney test, p=0.5334; Males—Vehicle 760±61.19 deg/cm, n=9; YB-537 762.2±58.17 deg/cm, n=9; unpaired t test, t=0.02672 df=16, p=0.9790; Females—Vehicle 651.4±54.55 deg/cm, n=8; YB-537 696.3±65.26 deg/cm, n=7; unpaired t test, t=0.5324 df=13, p=0.6035). (17F) A trend toward increased maximum movement bout duration of 5×FAD mice is seen when ingesting YB-537 (both sexes—Vehicle 5.996±0.238 s, n=17; YB-537 8.061±1.322 s, n=16; Mann-Whitney test, p=0.0643; Males 6.169±0.3541 s, n=9; YB-537±6.804 0.2919 s, n=9; unpaired t test, t=1.385 df=16, p=0.1851; Females—5.803±0.324 s, n=8; YB-537 9.677±3.012 s, n=7; Mann-Whitney test, p=0.2319). (17G) No change is seen in total movement duration of 5×FAD mice, regardless of sex (both sexes—Vehicle 244.9±3.544 s, n=17; YB-537 244.3±3.402 s, n=16; Mann-Whitney test, p=0.6827; Males—Vehicle 241.9±5.474 s, n=9; YB-537 243.4±4.5 s, n=9; unpaired t test, t=0.218 df=16, p=0.8302; Females—Vehicle 248.3±4.4 s, n=8; YB-537 245.5±5.568 s, n=7; unpaired t test, t=0.3967 df=13, p=0.6981). (17H) No change is seen in the proportion of time moving in 5×FAD mice, regardless of sex (both sexes—Vehicle 81.63±1.181%, n=17; YB-537 81.44±1.134%, n=16; Mann-Whitney test, p=0.6827; Males—Vehicle 80.62±1.825%, n=9; YB-537 81.13±1.5%, n=9; unpaired t test, t=0.218 df=16, p=0.8302; Females—Vehicle 82.76±1.467%, n=8; YB-537 81.84±1.856%, n=7; unpaired t test, t=0.3966 df=13, p=0.6981). (17I) No change is seen in hind-limb rearing frequency of 5×FAD mice, regardless of sex (both sexes—Vehicle 37.38±2.86, n=17; YB-537 35.72±3.292, n=16; unpaired t test, t=0.3828 df=31, p=0.7045; Males—Vehicle 36.39±3.666, n=9; YB-537 36.06±4.703, n=9; Mann-Whitney test, p=0.9125; Females—Vehicle 38.5±4.703, n=8; YB-537 35.29±4.894, n=7; unpaired t test, t=0.4727 df=13, p=0.06443). (17J) Nest quality was significantly correlated to time under treatment for the YB-537 group, but not with controls, regardless of sex (both sexes—Vehicle Pearson r, p=0.1392; YB-537 Pearson r, p=0.0241; Males—Vehicle Pearson r, p=0.2650; YB-537 Pearson r, p=0.0345; Females—Vehicle Pearson r, p=0.1056; YB-537 Pearson r, p=0.0173). Data are shown as mean±SEM. *p<0.05

FIGS. 18A-18L include graphs showing that ingestion of YB-537 in drinking water significantly improves cognitive function in 8-9 months old 5×FAD mice. (18A) Mice drinking YB-537 show a trend toward shorter escape latency in Morris water maze compared to controls (Two Way Repeated Measures ANOVA, Interaction F (5, 155)=1.78, p=0.1201; Time F (5, 155)=24.9, p<0.0001; Treatment F (1, 31)=3.539, p=0.0694; Subjects F (31, 155)=5.139, p<0.0001). (18B) Male mice receiving YB-537 in drinking water show insignificantly faster increments of spatial learning in the Morris water maze compared to controls (Two Way Repeated Measures ANOVA, Interaction F (5, 80)=1.063, p=0.3870; Time F (5, 80)=12.09, p<0.0001; Treatment F (1, 16)=2.837, p=0.1115; Subjects F (16, 80)=5.805, p<0.0001). (18C) Female mice receiving YB-537 in drinking water show insignificantly faster increments of spatial learning in the Morris water maze compared to controls (Two Way Repeated Measures ANOVA, Interaction F (5, 65)=1.095, p=0.3719; Time F (5, 65)=11.89, p<0.0001; Treatment F (1, 13)=0.6717, p=0.4272; Subjects F (13, 65)=4.14, p<0.0001). (18D) Mice receiving YB-537 in drinking water show a trend toward significantly discriminating a novel object, while control animals show no discrimination between the familiar and novel objects (Vehicle 0.0543±0.07876, n=17; YB-537 0.1785±0.08786, n=16; unpaired t test, t=1.055 df=31, p=0.2995; one sample t test Vehicle vs. 0, t=0.6894 df=16, p=0.5004; one sample t test YB-537 vs 0, t=2.032 df=15, p=0.0603). (18E) Male mice receiving YB-537 in drinking water and their controls show no discrimination between the familiar and novel objects (Vehicle 0.06542±0.1076, n=9; YB-537 0.06432±0.1285, n=9; unpaired t test, t=0.006588 df=16, p=0.9948; one sample t test Vehicle vs 0, t=0.6081 df=8, p=0.5600; one sample t test YB-537 vs 0, t=0.5006 df=8, p=0.6302). (18F) Female mice receiving YB-537 in drinking water are able to significantly discriminate a novel object from a familiar one, whilst control animals do not (Vehicle 0.04179±0.1233, n=8; YB-537 0.3253±0.09705, n=7; unpaired t test, t=1.768 df=13, p=0.1004; one sample t test Vehicle vs 0, t=0.3389 df=7, p=0.7447; one sample t test YB-537 vs 0, t=3.352 df=6, p=0.0154). (18G) Mice receiving YB-537 in drinking water freeze significantly more than controls in response to the conditioned context (Vehicle 23.78±3.357%, n=17; YB-537 34.93±2.513%, n=16; unpaired t test, t=2.634 df=31, p=0.0131). (18H) Male mice receiving YB-537 in drinking water freeze similarly to controls in response to the conditioned context (Vehicle 30.95±4.421%, n=9; YB-537 39.51±2.666%, n=9; unpaired t test, t=1.658 df=16, p=0.1168). (18I) Female mice receiving YB-537 in drinking water freeze significantly more than controls in response to the conditioned context (Vehicle 15.71±3.465%, n=8; YB-537 29.03±3.69%, n=7; unpaired t test, t=2.632 df=13, p=0.0207). (18J) Mice receiving YB-537 in drinking water freeze similarly to controls in response to the conditioned cue (Vehicle 13.56±2.409%, n=17; YB-537 16.47±1.689%, n=16; Mann-Whitney test, p=0.1000). (18K) Male mice receiving YB-537 in drinking water freeze similarly to controls in response to the conditioned cue (Vehicle 14.97±4.12%, n=9; YB-537 19.43±2.289%, n=9; Mann-Whitney test, p=0.0939). (18L) Female mice receiving YB-537 in drinking water freeze similarly to controls in response to the conditioned cue (Vehicle 11.97±2.385%, n=8; YB-537 12.66±1.739%, n=7; unpaired t test, t=0.2266 df=13, p=0.8243). Data are shown as mean±SEM; asterisks color-matched to plotted data represent within group post hoc tests. *p<0.05; **p<0.01.

FIGS. 19A-19E include graphs and fluorescent micrographs showing that drinking YB-537 for 1 month significantly reduces brain pathologies associated with dementia in 8-9 months old 5×FAD mice. (19A) Oxidative stress, as indicated by 4-HNE volume normalized to corresponding μm³ of brain, in the CA1 of 8-9 month old 5×FAD mice is not significantly altered following 1 month drinking of YB-537, but trends to significantly alter distribution in both total and male populations, while significantly changing distribution in females, by reducing or removing extreme high and low measurements of 4-HNE volume (both sexes—Vehicle 0.053±0.005, n=16; YB-537 0.049±0.003, n=16; Mann-Whitney test, p=0.8965; Males—Vehicle 0.055±0.007, n=8; YB-537 0.053±0.005, n=9; Mann-Whitney test, p=0.6730; Females—Vehicle 0.055±0.007, n=8; YB-537 0.053±0.005, n=9; unpaired t test, t=0.2375 df=15, p=0.8155). (19B) Amyloid R volume normalized to μm³ of brain measured in CA1 of 8-9 month old 5×FAD mice is significantly reduced following 1 month drinking of YB-537 in the total population (Vehicle 0.049±0.0031, n=17; YB-537 0.040±0.003, n=16; unpaired t test, t=2.015 df=31, p=0.0526) is insignificantly reduced in the male population (Vehicle 0.041±0.002, n=9; YB-537 0.038±0.004, n=9; un[aired t test, t=0.5887 df=16, p=0.5643) and significantly reduced in the female population (Vehicle 0.059±0.003, n=8; YB-537 0.044±0.004, n=7; unpaired t test, t=2.56 df=13, p=0.0237). (19C) Volume of p-Tau normalized to μm³ of brain measured in the CA1 of 8-9 month old 5×FAD mice is unchanged following 1 month drinking of YB-537 in the total (Vehicle 0.054±0.004, n=17; YB-537 0.04±8 0.006, n=16; unpaired t test, t=0.7351 df=31, p=0.4678) and male (Vehicle 0.055±0.006, n=9; YB-537 0.064±0.006, n=9; unpaired t test, t=0.9768 df=16, p=0.3432) populations, but is significantly reduced in the female population (Vehicle 0.052±0.008, n=8; YB-537 0.027±0.006, n=7; unpaired t test, t=2.373 df=13, p=0.0337). (19D) Volume of Iba1 normalized to μm³ of brain measured in the CA1 of 8-9 month old 5×FAD mice is insignificantly changed following 1 month drinking of YB-537 in the total population (Vehicle 0.039±0.002, n=17; YB-537 0.032±0.002, n=16; unpaired t test, t=1.851 df=31, p=0.0738), is unchanged in the male population (Vehicle 0.040±0.002, n=9; YB-537 0.038±0.003, n=9; unpaired t test, t=0.5915 df=16, p=0.5625), but is significantly reduced in the female population (Vehicle 0.037±0.004, n=8; YB-537 0.025±0.002, n=7; unpaired t test, t=2.309 df=13, p=0.0380). (19E) The sum of the intensity of Iba1 positive signal normalized to μm³ of brain measured in the CA1 of 8-9 month old 5×FAD mice is insignificantly changed following 1 month drinking of YB-537 in the total population (Vehicle 77.81±5.876, n=17; YB-537 63.57±5.251, n=16; unpaired t test, t=1.799 df=31, p=0.0817), is unchanged in the male population (Vehicle 76.1±7.494, n=9; YB-537 74.09±6.198, n=9; unpaired t test, t=0.2065 df=16, p=0.8390), but is significantly reduced in the female population (Vehicle 79.74±9.722, n=8; YB-537 50.04±6.123, n=7; unpaired t test, t=2.496 df=13, p=0.0268).

FIGS. 20A-20D include fluorescent micrographs showing representative images from female 5×FAD mice following 1 month of YB-537 or vehicle ingestion. (20A) 4-HNE (green) and DAPI (blue) as imaged from 8-9 months old female 5×FAD mice hippocampal CAL. (20B) Amyloid R (green) and DAPI (blue) as imaged from 8-9 months old female 5×FAD mice hippocampal CAL. (20C) Phosphorylated Tau (red) and DAPI (blue) as imaged from 8-9 months old female 5×FAD mice hippocampal CAL. (20D) Iba1 (red) and DAPI (blue) as imaged from 8-9 months old female 5×FAD mice hippocampal CA1.

FIGS. 21A-21D include fluorescent micrographs showing representative images from male 5×FAD mice following 1 month of YB-537 or vehicle ingestion. (21A) 4-HNE (green) and DAPI (blue) as imaged from 8-9 months old male 5×FAD mice hippocampal CAL. (21B) Amyloid R (green) and DAPI (blue) as imaged from 8-9 months old male 5×FAD mice hippocampal CAL. (21C) Phosphorylated Tau (red) and DAPI (blue) as imaged from 8-9 months old male 5×FAD mice hippocampal CAL. (21D) Iba1 (red) and DAPI (blue) as imaged from 8-9 months old male 5×FAD mice hippocampal CA1.

### DETAILED DESCRIPTION

According to some embodiments, there is provided a pharmaceutical composition for use in the treatment or prevention of a quinone reductase 2 (QR2)-related disease or disorder in a subject in need thereof, comprising the compound represented by or comprises Formula 1 or any salt thereof.

In one aspect of the invention, there is a compound represented by Formula 1:

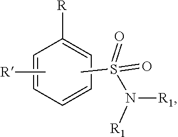

wherein:
R′ is selected from

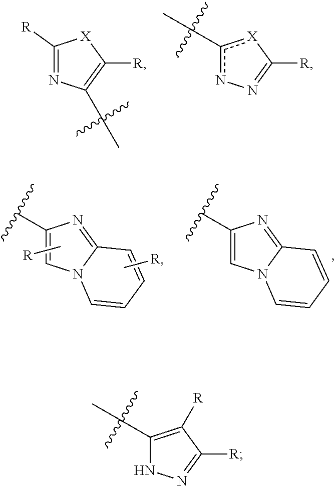

each R independently represents hydrogen, or a substituent comprising halogen, —NO₂, —CN, —OH, —CONH₂, —CONR″₂, —CNNR″₂, —CSNR″₂, —CONH—OH, —CONH—NH₂, —NHCOR″, —NHCSR″, —NHCNR″, —NC(═O)OR″, —NC(═O)NR″, —NC(═S)OR″, —NC(═S)NR″, —SO₂R″, —SOR″, —SR″, —SO₂OR″, —SO₂N(R″)₂, —NHNR″₂, —NNR″, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —C(═O)N(C₁-C₆ alkyl)₂, —CO₂H, —CO₂R″, —OCOR″, —OCOR″, —OC(═O)OR″, —OC(═O)NR″, —OC(═S)OR″, —OC(═S)NR″, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; wherein R″ represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof;
[symbol] represents a single or a double bond;
each X independently comprises C, CH, S, O, N or NH as allowed by valency;
and each R1 independently is H or comprises optionally substituted C₁-C₆ alkyl, hydroxy(C₁-C₆ alkyl), C₁-C₆ haloalkyl, optionally substituted C₃-C₈ cycloalkyl, optionally substituted C₁-C₆ alkyl-cycloalkyl, optionally substituted C₁-C₆ alkyl-aryl, optionally substituted C₁-C₆ alkyl-heteroaryl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or both R1 are interconnected, so as to form a cyclic ring. In some embodiments, the compound of the invention is represented by Formula 1, wherein both R1 are interconnected, so as to form a cyclic ring (e.g., 3-7, 3, 4, 5, 6, 7-membered aliphatic ring, heteroaromatic ring and/or an aliphatic ring optionally comprising a heteroatom), a bicyclic ring and/or fused ring. In some embodiments, the compound of the invention including any salt thereof, is represented by Formula 1. In some embodiments, the cyclic ring is devoid of a bicyclic ring and/or of a fused ring. In some embodiments, at least one X is not C or CH (e.g. is a heteroatom).

In some embodiments, the compound of the invention is represented by Formula 1, wherein R and

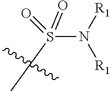

are located in ortho position, in para position or in meta position to each other. In some embodiments, the compound of the invention is represented by Formula 1, wherein R and

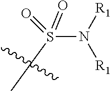

are located in in para position or in meta position to each other.

In some embodiments, the compound of the invention is represented by Formula 1′

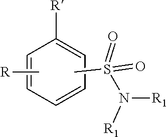

wherein R′, R, and R1 are as described herein.

In some embodiments, the compound of the invention is represented by Formula 1, or by Formula 1′, wherein R′ is selected from

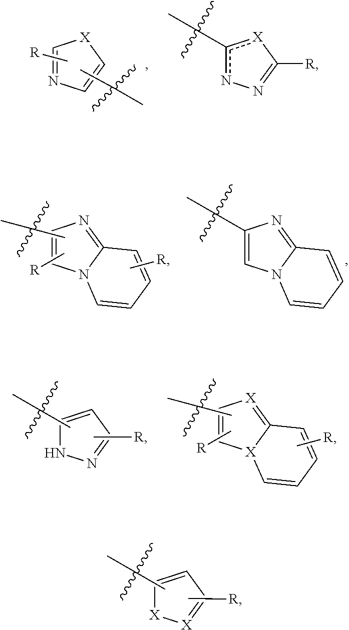

wherein R and X are as described herein. In some embodiments, each R represents one or more substituents. In some embodiments, R represents at least two substituents, optionally wherein the substituents are interconnected so as to form a 5-7 membered ring.

In some embodiments, the compound of the invention is represented by Formula 1A:

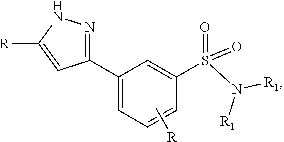

### or by Formula 1A1:

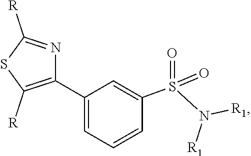

wherein R and R1 are as described herein.

In some embodiments, the compound of the invention is represented by Formula 1A or by Formula 1A1, wherein each R is or comprises optionally substituted C₁-C₆ alkyl. In some embodiments, each R1 independently represents optionally substituted C₁-C₆ alkyl, optionally substituted C₁-C₆ alkyl, optionally substituted C₁-C₆ alkyl-cycloalkyl, optionally substituted C₁-C₆ alkyl-heterocyclyl, optionally substituted C₁-C₆ alkyl-aryl, optionally substituted C₁-C₆ alkyl-heteroaryl. In some embodiments, any one of aryl, heterocyclyl, heteroaryl, and the cycloalkyl comprises one or more substituents, as described herein. In some embodiments, the cycloalkyl optionally comprises one or more heteroatoms selected from N, NH, O, and S, as allowed by valency. In some embodiments, the cycloalkyl is or comprises a lactam (e.g. a beta-, gamma-, or delta-lactam).

In some embodiments, the compound of the invention is represented by Formula 1B:

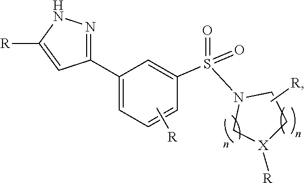

### or by Formula 1B1:

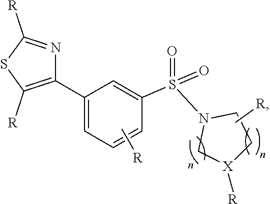

wherein R is as described herein, X comprises CH, CH2, S, O, N or NH; and each n independently represents an integer between 1 and 3 (e.g. 1, 2, or 3).

In some embodiments, the compound of the invention is or comprises any one of:

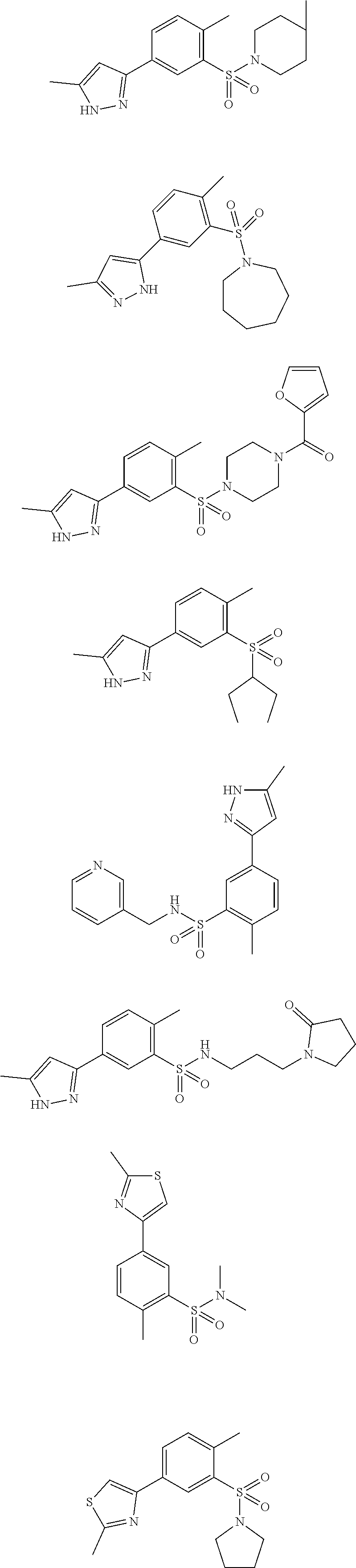

In some embodiments, the compound of the invention is represented by Formula 1C:

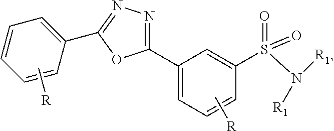

wherein R is as described herein, and wherein each R1 independently comprises optionally substituted C₁-C6 alkyl, hydroxy(C1-C6 alkyl), C₁-C6 haloalkyl, optionally substituted C₃-C8 cycloalkyl, optionally substituted C₁-C6 alkyl-cycloalkyl, optionally substituted C₁-C6 alkyl-aryl, optionally substituted C₁-C6 alkyl-heteroaryl, optionally substituted C₃-C8 heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or both R1 are interconnected, so as to form a cyclic ring. In some embodiments, the compound of the invention is represented by Formula 1C, wherein both R1 are interconnected, so as to form a cyclic ring (e.g., 3-7, 4, 5, 6-membered aliphatic ring, heteroaromatic ring and/or an aliphatic ring optionally comprising a heteroatom), a bicyclic ring and/or fused ring.

In some embodiments, the compound of the invention is represented by Formula 1C, wherein each R is or comprises optionally substituted C₁-C6 alkyl. In some embodiments, each R1 independently represents optionally substituted C₁-C6 alkyl, optionally substituted C1-C6 alkyl, optionally substituted C₁-C6 alkyl-cycloalkyl, optionally substituted C₁-C6 alkyl-heterocyclyl, optionally substituted C₁-C6 alkyl-aryl, optionally substituted C₁-C6 alkyl-heteroaryl.

In some embodiments, the compound of the invention is represented by Formula 1D:

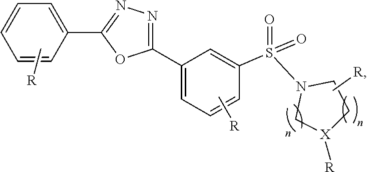

wherein R is as described herein, X comprises CH, CH2, S, O, N or NH; and n is an integer between 1 and 3.

In some embodiments, the compound of the invention is or comprises any one of:

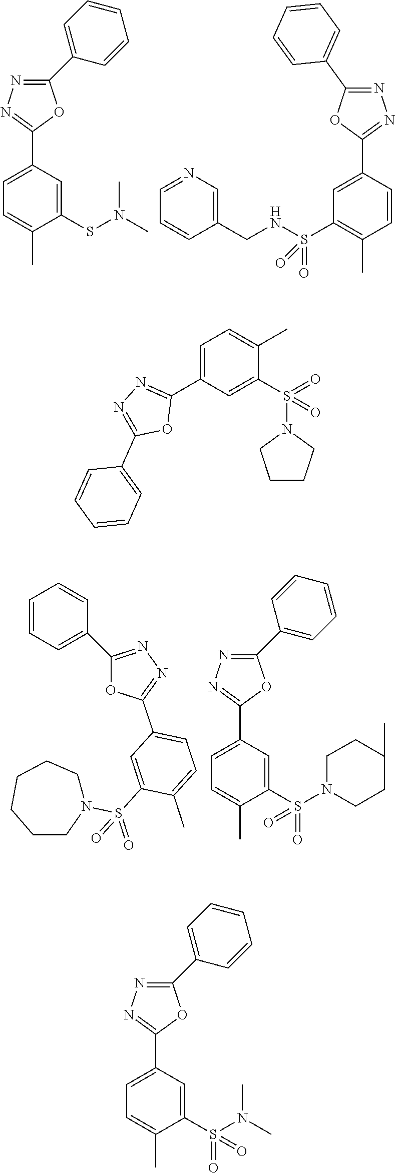

In some embodiments, the compound of the invention is represented by Formula 2:

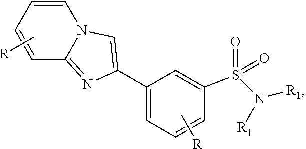

wherein R and R1 are as described herein.

In some embodiments, the compound of the invention is represented by Formula 2, wherein each R is or comprises optionally substituted C₁-C₆ alkyl. In some embodiments, each R1 independently represents optionally substituted C₁-C₆ alkyl, optionally substituted C₁-C₆ alkyl, optionally substituted C₁-C₆ alkyl-cycloalkyl, optionally substituted C₁-C₆ alkyl-heterocyclyl, optionally substituted C₁-C₆ alkyl-aryl, optionally substituted C₁-C₆ alkyl-heteroaryl. In some embodiments, each R1 independently represent optionally substituted heteroaryl, optionally substituted aryl, optionally substituted cycloalkyl, optionally substituted heterocyclyl, optionally substituted bicyclic heteroaryl.

In some embodiments, the compound of the invention is represented by Formula 3:

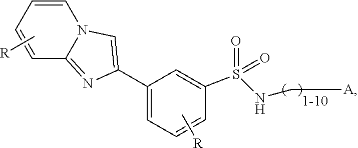

wherein each R is as described herein, and A is or comprises optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof.

In some embodiments, the compound of the invention is represented by Formula 4:

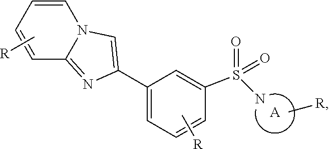

wherein A and R are as described herein. In some embodiments, A comprises an optionally substituted 3-8 membered ring. In some embodiments, A comprises an optionally substituted 3-8 membered ring comprising 1, 2, 3, or 4 heteroatoms (e.g. O, N, S). In some embodiments, A comprises 2 or more heteroatoms selected from N, NH, and S, as allowed by valency. In some embodiments, R is bound to A via a carbon atom, or via N. In some embodiments, A is selected from the group comprising pyrrolidine, oxirane, tetrahydrofuran, aziridine, pyran, dioxane, thiolane, oxathiolane, piperidine, and/or morpholine. In some embodiments, R is attached to a C—, or to a heteroatom of A.

In some embodiments, the compound of the invention is represented by Formula:

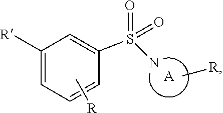

### or by Formula:

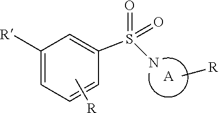

wherein R, R′, R1 and A are as described herein, and wherein R1 is not H.
In some embodiments, the compound of the invention is represented by Formula:

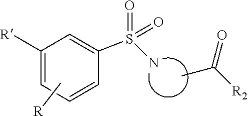

### or by Formula:

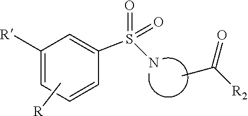

wherein R, R′, R1 and A are as described herein, and wherein R1 is not H; and wherein R2 is or comprises optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof.

In some embodiments, the compound of the invention is represented by any one of Formulae:

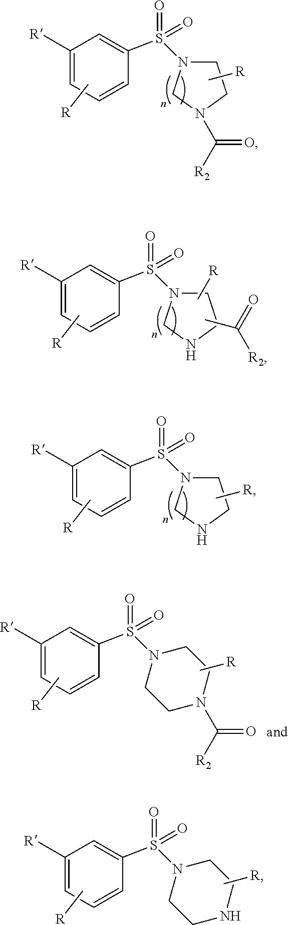

wherein n, R, R2 and R′ are as described herein.

In some embodiments, the compound of the invention is represented by Formula 4A:

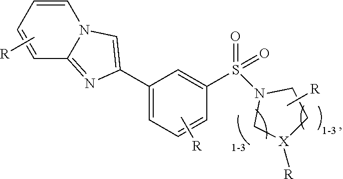

wherein R is described herein, and X comprises CH, CH2, S, O, N or NH. In some embodiments, X comprises S, N or NH.

In some embodiments, the compound of the invention is represented by Formula:

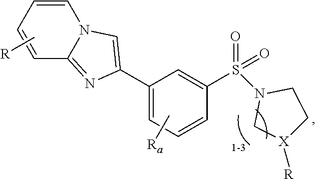

wherein R and X are as described herein, and Ra represents a substituent comprising halogen, —NO₂, —CN, —OH, —CONH₂, —CONR″₂, —CNNR″₂, —CSNR″₂, —CONH—OH, —CONH—NH₂, —NHCOR″, —NHCSR″, —NHCNR″, —NC(═O)OR″, —NC(═O)NR″, —NC(═S)OR″, —NC(═S)NR″, —SO₂R″, —SOR″, —SR″, —SO₂OR″, —SO₂N(R″)₂, —NHNR″₂, —NNR″, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —C(═O)N(C₁-C₆ alkyl)₂, —CO₂H, —CO₂R″, —OCOR″, —OCOR″, —OC(═O)OR″, —OC(═O)NR″, —OC(═S)OR″, —OC(═S)NR″, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; wherein R″ represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof as allowed by valency.

In some embodiments, the compound of the invention is represented by Formula 4B:

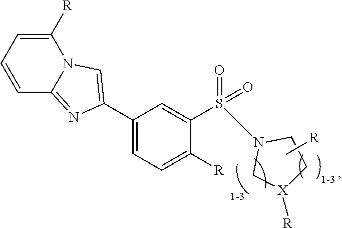

wherein R and X are as described herein.

In some embodiments, the compound of the invention is represented by Formula 4C:

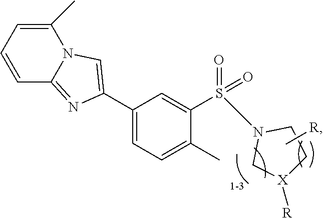

wherein R and X are as described herein.

In some embodiments, the compound of the invention is represented by Formula 5:

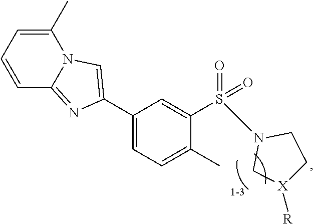

wherein R and X are as described herein. In some embodiments, X is N or NH. In some embodiments, R is or comprises optionally substituted C₁-C₆ alkyl. In some embodiments, R is or comprises —C(═O)R3, —C(═N)R3, —C(═O)OR3, —C(═O)NR₃, —C(═S)OR3, —C(═S)NR₃, wherein R3 represents hydrogen, or is selected from the group comprising optionally substituted C₁-C₆ alkyl, optionally substituted C₃-C₁₀ cycloalkyl, optionally substituted C₃-C₁₀ heterocyclyl, optionally substituted heteroaryl, optionally substituted aryl or a combination thereof.

In some embodiments, the compound of the invention is represented by Formula 6:

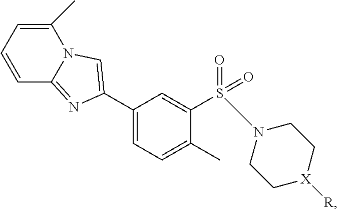

wherein R is as described herein.

In some embodiments, R is H.

In some embodiments, the compound of the invention is or comprises any one of including any salt and/or derivative thereof:

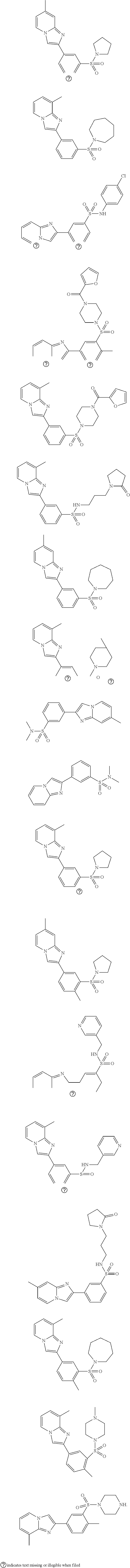

In some embodiments, the compound of the invention is represented by Formula:

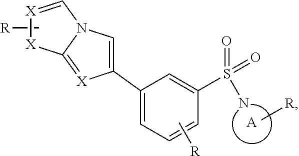

### or by Formula

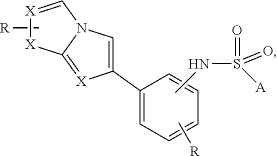

### or by Formula

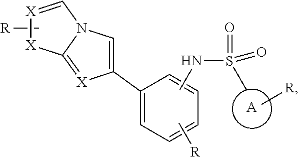

### or by Formula

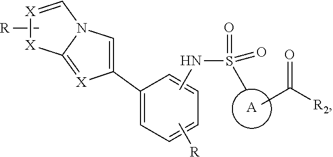

wherein X, A and R are as described herein, or wherein A is absent. In some embodiments, at least one X is a heteroatom.

In some embodiments, the compound of the invention is represented by Formula:

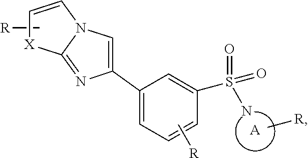

### or by Formula

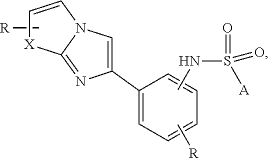

wherein X, A and R are as described herein, or wherein A is absent.

In some embodiments, the compound of the invention is represented by Formula:

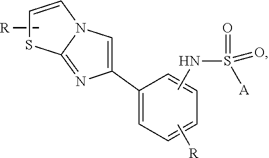

wherein A and R are as described herein.

In some embodiments, the compound of the invention is represented by Formula:

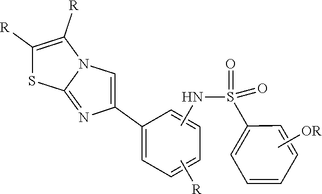

### or by Formula:

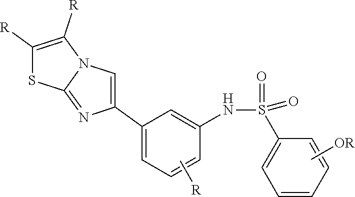

wherein R is as described herein.

Several compounds of the invention showed significant Quinone Reductase 2 (QR2) inhibition in-vitro, showing micromolar and sub-micromolar IC₅₀ values. Some of the compounds of the invention (e.g. characterized by significant QR2 inhibitory activity are presented in Table 3 below). Some of the selected compounds exhibited IC₅₀ values between 1 and 10 nM.

As used herein, the term “substituted” comprises one or more substituents (e.g., between 1 and 2, between 2 and 3, between 3 and 4, between 4 and 5, substituents including any range or value therebetween), wherein each substituent independently comprises (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, —NO₂, —CN, —OH, —CONH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, -oxy (i.e. ═O), or a combination thereof.

As used herein, the term “7-10 ring” is referred to a cyclic aliphatic or aromatic compound comprising between 7 and 10 carbon atoms. In some embodiments, 7-10 ring bicyclic ring comprises between 7 and 8, between 8 and 9, between 9 and 10 carbon atoms including any value therebetween.

As used herein the term “C₁-C₆ alkyl” including any C₁-C₆ alkyl related compounds, is referred to any linear or branched alkyl chain comprising between 1 and 6, between 1 and 2, between 2 and 3, between 3 and 4, between 4 and 5, between 5 and 6, carbon atoms, including any range therebetween. In some embodiments, C₁-C₆ alkyl comprises any of methyl, ethyl, propyl, butyl, pentyl, iso-pentyl, hexyl, and tert-butyl or any combination thereof. In some embodiments, C₁-C₆ alkyl as described herein further comprises an unsaturated bond, wherein the unsaturated bond is located at 1^{st}, 2^{nd}, 3^{rd}, 4^{th}, 5^{th}, or 6^{th} position of the C₁-C₆ alkyl.

As used herein the term “(C₃-C₁₀) cycloalkyl” is referred to an optionally substituted C₃, C₄, C₅, C₆, C₇, C₈, C₉ or C₁₀ ring. In some embodiments, (C₃-C₁₀) ring comprises optionally substituted cyclopropane, cyclobutene, cyclopentane, cyclohexane, or cycloheptane.

As used herein the term “(C₃-C₅) cycloalkyl” is referred to an optionally substituted C₃, C₄, C₅, C₆, C₇, or C₈ ring. In some embodiments, (C₃-C₁₀) ring comprises optionally substituted cyclopropane, cyclobutene, cyclopentane, cyclohexane, or cycloheptane.

As used herein the term “(C₆-C₁₂) ring” is referred to an optionally substituted C₆, C₇, C₈, C₉, C₁₀, C₁₁, or C₁₂ ring. In some embodiments, (C₆-C₁₂) ring is referred to a bicyclic ring (e.g. fused ring, spirocyclic ring, biaryl ring).

As used herein the term “bicyclic heteroaryl” referred to (C₆-C₁₂) a bicyclic heteroaryl ring, wherein bicyclic (C₆-C₁₀) ring is as described herein.

As used herein the term “bicyclic aryl” referred to (C₆-C₁₂) a bicyclic aryl ring, wherein bicyclic (C₆-C₁₂) ring is as described herein.

As used herein the term “bicyclic heterocyclyl” referred to (C₆-C₁₂) a bicyclic heterocyclic ring, wherein (bicyclic C₆-C₁₂) ring is as described herein.

As used herein the term “bicyclic cycloalkyl” referred to (C₆-C₁₂) a bicyclic cycloalkyl ring, wherein bicyclic (C₆-C₁₂) ring is as described herein.

In some embodiments, the compound of the invention comprises any one of the compounds disclosed herein, including any salt thereof. In some embodiments, the salt of the compound is a pharmaceutically acceptable salt.

In some embodiments, there is provided a composition comprising the compound of the invention, and an acceptable carrier.

As used herein, the terms or compounds referred to “YB-537“and” NG-537” are synonymous. As used herein, the terms or compounds referred to “YB-800“and” NG-800” are synonymous. As used herein, the terms or compounds referred to “YB-808“and” NG-808” are synonymous.

### Pharmaceutical Composition

According to some embodiments, there is provided a pharmaceutical composition for use in the treatment or prevention of a quinone reductase 2 (QR2)-related disease or disorder in a subject in need thereof, comprising the compound represented by or comprises Formula 1 as disclosed herein, or any salt thereof.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, nitrate, phosphate, malonate, citrate, salicylate, stearate, succinate, sulfate, tartrate, decanoate, edetate, fumarate, gluconate, and lactate including any protonated specie, or any combination thereof.

In some embodiments, the pharmaceutical composition comprises the compound of the invention and a pharmaceutically acceptable carrier. In some embodiments, the pharmaceutical composition comprises a therapeutically effective amount of the compound of the invention and the pharmaceutically acceptable carrier.

For example, the term “pharmaceutically acceptable” can mean approved by a regulatory agency of the Federal or a state government or listed in the U.S. Pharmacopeia or other generally recognized pharmacopeia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for a systemic use.

In some embodiments, the pharmaceutical composition is any of an emulsion, a liquid solution, a gel, a paste, a suspension, a dispersion, an ointment, a cream, or a foam.

As used herein, the term “carrier” refers to a diluent, adjuvant, excipient, or vehicle with which the active ingredient is administered. Such carriers can be sterile liquids, such as water-based and oils, including those of petroleum, animal, vegetable, or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, Eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate, that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has a sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition is a liquid at a temperature between 15 to 45° C. In some embodiments, the pharmaceutical composition is a solid at a temperature between 15 to 45° C. In some embodiments, the pharmaceutical composition is a semi-liquid at a temperature between 15 to 45° C. It should be understood that the term “semi-liquid”, is intended to mean materials which are flowable under pressure and/or shear force. In some embodiments, semi-liquid compositions include creams, ointments, gel-like materials, and other similar materials. In some embodiments, the pharmaceutical composition is a semi-liquid composition, characterized by a viscosity in a range from 31,000-800,000 cps.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical composition being in the form of a cream further comprises a thickener.

Non-limiting examples of thickeners include, but are not limited to microcrystalline cellulose, a starch, a modified starch, gum tragacanth, gelatin, and a polymeric thickener (e.g., polyvinylpyrrolidone) or any combination thereof.

In some embodiments, the pharmaceutical composition comprising the compound of the invention is in a unit dosage form. In some embodiments, the pharmaceutical composition is prepared by any of the methods well known in the art of pharmacy. In some embodiments, the unit dosage form is in the form of a tablet, capsule, lozenge, wafer, patch, ampoule, vial, or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral, or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition is administered via oral (i.e., enteral), rectal, vaginal, topical, sublingual, buccal, nasal, ophthalmic, transdermal, subcutaneous, intramuscular, intraperitoneal, intrathecal, or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the pharmaceutical composition is in a form of, for example, and not by way of limitation, an ointment, cream, gel, paste, foam, aerosol, suppository, pad, or gelled stick.

In some embodiments, for oral applications, the pharmaceutical composition is in the form of a tablet or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous, and intraperitoneal injection purposes.

In some embodiments, the pharmaceutical composition is for use in the treatment of a QR2-related disease or disorder.

In some embodiments, compounds of the invention inhibit 50% of QR2 activity at a concentration (e.g., IC50) of less than 500 μM, less than 400 μM, less than 150 μM, less than 50 μM, less than 200 μM, less than 10000 nM, less than 5000 nM, less than 2000 nM, less than 1000 nM, less than 200 nM, less than 500 nM, less than 50 nM, less than 10 nM, less than 5 nM, less than 1 nM. In some embodiments, inhibition of QR2 activity is evaluated in-vitro.

In some embodiments, a QR2 protein comprises the amino acid sequence:
(SEQ ID NO: 1)
MAGKKVLIVYAHQEPKSFNGSLKNVAVDELSRQGCTVTVSDLYAMNLEPRATDK
DITGTLSNPEVFNYGVETHEAYKQRSLASDITDEQKKVREADLVIFQFPLYWFSVP
AILKGWMDRVLCQGFAFDIPGFYDSGLLQGKLALLSVTTGGTAEMYTKTGVNGD
SRYFLWPLQHGTLHFCGFKVLAPQISFAPEIASEEERKGMVAAWSQRLQTIWKEEP
IPCTAHWHFGQ.
In some embodiments, a QR1 protein comprises the amino acid sequence:
(SEQ ID NO: 2)
MVGRRALIVLAHSERTSFNYAMKEAAAAALKKKGWEVVESDLYAMNFNPIISRK
DITGKLKDPANFQYPAESVLAYKEGHLSPDIVAEQKKLEAADLVIFQFPLQWFGVP
AILKGWFERVFIGEFAYTYAAMYDKGPFRSKKAVLSITTGGSGSMYSLQGIHGDM
NVILWPIQSGILHFCGFQVLEPQLTYSIGHTPADARIQILEGWKKRLENIWDETPLYF
APSSLFDLNFQAGFLMKKEVQDEEKNKKFGLSVGHHLGKSIPTDNQIKARK.

### Methods of Use

In another aspect, there provided herein is a method for preventing or treating a QR2-related disease or disorder in a subject in need thereof.

As used herein, the term “QR2-related disease or disorder” refers to any disease, disorder, or condition involving QR2 function and/or activity in the pathogenesis and/or pathophysiology of the disease, disorder, or condition.

As used herein, the term “QR2 function or activity” refers to quinone reduction. In some embodiments, QR2 function or activity is co-factor dependent. In some embodiments, the co-factor comprises or is dihydronicotinamide riboside (NRH) or other nicotinamide molecules.

In some embodiments, QR2 function or activity comprises reduction of quinones to be nontoxic.

QR2 is a flavoprotein enzyme that catalyzes the reduction of quinones using NRH. The molecular weight of QR2 is about 26,000 Da. It comprises a homodimer (comprising of two identical structures), each consisting of 231 amino acids, each of which contains flavin adenine dinucleotide (FAD) in its active site. It is also called menadione reductase, vitamin K reductase (vitamin K reductase), DT-diaphorase (DT-diaphorase), NQO2, Ribosyldihydronicotinamide dehydrogenase [quinone], NRH dehydrogenase [quinone] 2, NRH:quinone oxidoreductase 2 and the like.

Quinone reductase has several characteristics. First, NRH is used as an electron (hydride) donor, and secondly, it is able to reduce a wide variety of substrates by 1, 2 or 4 electrons to varying outcomes that can be ROS generating or toxifying, and thirdly, it is strongly inhibited by flavonoids such as resveratrol and quercetin and is fourthly induced by various foreign substances in many cells and tissues.

QR2 is over expressed in the ageing brain and in Alzheimer's (AD) and Parkinson's (PD) diseases, where it contributes to cognitive decline and generates reactive oxygen species (ROS). The inability to reduce QR2 expression prevents QR2 pathway activation, necessary for normal memory, and also causes a net increase in oxidative stress. QR2 inhibition improves cognition and reduces ROS generation, therefore targeting both brain pathogenesis and cognitive deficits associated with age related neurodegeneration.

QR2 is highly expressed in many types of cancer, is positively associated with worsening prognosis, and is involved in cancer cell proliferation and redox state, while it is an adventitious off-target for multiple anti-cancer drugs. QR2 inhibition therefore poses a promising avenue for cancer therapy, and prevention of cancerous growth, activation, and metastasis.

Anti-malarial quinolines and quinolones are non-specific QR2 inhibitors, suggesting QR2 inhibition may aid against malaria plasmodium infection, as has been previously shown with anti-malaria drug derived QR2 inhibitors.

QR2 causes toxification of certain substrates, such as mitomycin C, CB1954 and menadione, while it generates ROS via reduction of ortho-quinones, including monoamine quinones, as well as acetaminophen. Inhibition of QR2 prior to administration of certain drugs may, therefore, confer protection from the drug's side effects, the most pressing example being acute liver failure caused by acetaminophen and NAPQI.

The endogenous co-factor of QR2 is dihydronicotinamide riboside (NRH), the QR2 oxidation of which has been shown to affect the rate of NAD production, which is central to cell metabolism and function. Additionally, QR2 oxidation of NRH drives 4-pyridone-3-carboxamide-1-β-D-ribonucleoside (4PYR) product generation, which is a toxic pyridone associated with aging metabolism and renal failure. QR2 inhibition can therefore be used to correctively effect aforementioned NRH and related NAD metabolism, and can also be used to attenuate toxic outcomes of NAD and/or 4PYR overproduction.

Melatonin inhibits QR2, and the related anti-epileptic drug VLB-01 (Beprodone) binds QR. QR2 inhibition may therefore aid in attenuating epilepsy.

Inhibition of QR2 has shown neuroprotective and cardioprotective effects against ischemia-reperfusion injury following cardiopulmonary bypass and deep hypothermic circulatory arrest.

The multi-target anxiolytic drug afobazole and its active metabolite M-11 bind and inhibit QR2, conferring both neuroprotective effects against PD and ischemia as well as depression. Melatonin derivatives that bind and inhibit QR2 preferentially over other melatonin receptors confer anti-depressant effects.

Genetic QR2 removal reduces neointimal formation following arterial injury, reducing the risk of the development of restenosis. QR2 inhibition may therefore result in similar outcomes.

MCA-NAT is a non-soluble melatonin derivative and anti-depressant that selectively binds QR2 and has been experimentally shown to reduce intra-ocular pressure associated with glaucoma, presumably via blockage of chloride channels.

QR2 expression is reduced following adenovirus infection as part of transcriptional changes required for acute-phase and adaptive immune response, while increased QR2 expression is correlated with fast viral progression in vivo.

According to some embodiments, there is provided a method for inhibiting or reducing the function or activity of QR2 in a cell or a subject.

In some embodiments, the method comprises administering a therapeutically effective amount of the pharmaceutical composition of the invention to a subject.

In some embodiments, the method comprises contacting a cell with an effective amount of the compound of the invention, or a composition comprising same.

In some embodiments, treating comprises ameliorating at least one symptom associated with the QR2-related disease or disorder.

In some embodiments, a subject suitable for the treatment disclosed herein is characterized by an abnormal expression of QR2. In some embodiments, the abnormal expression is an increased expression compared to a control subject. In some embodiments, abnormal comprises dysregulated, upregulated, unregulated, or any combination thereof.

In some embodiments, the disease or disorder is selected from: a neurodegenerative disorder, a cell-proliferative disease or disorder, malaria, drug direct or indirect toxicity, NAD-related metabolic toxicity, epilepsy, ischemia, depression and/or anxiety, restenosis, glaucoma, immune-response related disease.

As used herein, direct toxicity refers to the drug being a toxic agent per se to a cell or an organism.

As used herein indirect toxicity refers to the drug being metabolized by a cell or an organism, wherein one or more intermediates or end products of the drug metabolism being toxic to the cell or the organism.

In some embodiments, a cell-proliferative disease comprises any one of cancer and inflammation.

In some embodiments, the disease or disorder is or comprises: a ROS-related disease, an autophagy related disease, an inflammatory disease or disorder.

As used herein, “ROS-related disease” refers to any disease, disorder, or condition involving or characterized by ROS involvement in the pathogenesis and/or pathophysiology of the disease, disorder, or condition.

As used herein, “autophagy-related disease” refers to any disease, disorder, or condition involving or characterized by autophagy involvement in the pathogenesis and/or pathophysiology of the disease, disorder, or condition.

In some embodiments, autophagy-related disease is characterized by excessive, abnormal, abnormally increased, upregulated, or any combination thereof, autophagy.

In some embodiments, administering is by an oral administration, a topical administration, a systemic administration, or a combination thereof.

In some embodiments, the neurological disorder to be treated with a compound and/or a composition as described herein, is selected from: multiple sclerosis, Alzheimer's Disease, dementia, Parkinson's disease, Huntington's disease, Down syndrome, Amyotrophic lateral sclerosis (ALS), brain injury, stroke, ischemic reperfusion, and prion disease or any combination thereof and acquired and inherited neuropathies in the peripheral nervous system. In some embodiments, the neurological disorder is multiple sclerosis. In some embodiments, the neurological disorder is Alzheimer's disease.

In some embodiments, treating or ameliorating comprises any one of: improving cognitive function of the subject, inhibiting cognitive dysfunction of the subject, and any combination thereof.

As used herein, the term “cognitive function” is well-known in the art and refers to multiple mental abilities, including learning, thinking, reasoning, remembering, problem solving, decision making, and attention.

In some embodiments, the method comprises inducing neuroprotective effect of a nerve cell and/or glia cell of the subject.

In some embodiments, the disease comprises brain injury, stroke, ischemic reperfusion, or any combination thereof. In some embodiments, the brain injury, stroke, ischemic reperfusion, or any combination thereof is induced by trauma. In some embodiments, the herein disclosed method is for treating or preventing brain injury (TBI), stroke, ischemic reperfusion, or any combination thereof induced by trauma.

According to some embodiments, there is provided a method for: (a) treating SARS or SARS-CoV-2 infection; or (b) inhibiting or reducing cytokine-induced inflammation or neuroinflammation comprising admistering to a subject afflicted with inflammation or neuroinflammation a compound represented by or comprises Formula 1

In some embodiments, the disease or disorder comprises an infectious disease. In some embodiments, the infectious disease comprises a viral disease. In some embodiments, the infectious disease is induced by a virus. In some embodiments, the virus comprises a coronavirus. In some embodiments, the virus induces Coronavirus disease 2019 (COVID-19). In some embodiments, the disease is SARS (Severe Acute Respiratory Syndrome) or SARS-CoV-2 disease or infection. In some embodiments, the disease is or comprises COVID-19. In some embodiments, the infectious disease induces, promotes, propagates, or any combination thereof inflammation, neuroinflammation, or both. In some embodiments, inflammation or neuroinflammation is cytokine-induced inflammation or neuroinflammation.

In some embodiments, the herein disclosed method is directed to treating SARS or SARS-CoV-2 infection.

In some embodiments, the herein disclosed method is directed to inhibiting or reducing cytokine-induced inflammation or neuroinflammation.

In some embodiments, the herein disclosed method is for treating or preventing a mitochondria-related disease. In some embodiments, the mitochondria-related disease is characterized by disrupted mitochondria or disrupted mitochondrial activity, mitochondrial loss of function, mitochondrial dysfunction, or any combination thereof. In some embodiments, mitochondrial-related disease is characterized by inhibited or reduced oxidative phosphorylation (OXPHOS). In some embodiments, the mitochondrial-related disease is induced or caused by an infection, a toxin, or both.

In some embodiments, the treating comprises activating or enabling OXPHOS, increasing OXPHOS rate, or both.

As used herein, “mitochondrial-related disease” refers to any disease, disorder, or condition characterized by reduced, inhibited, disrupted, abnormal mitochondria or activity thereof, involvement in the pathogenesis and/or pathophysiology of the disease, disorder, or condition.

In some embodiments, mitochondrial-related disease is a genetic mitochondrial-related disease. In some embodiments, a genetic mitochondrial-related disease comprises at least one mutation or any genetic aberration of the nuclear genome or in a nuclear gene which induces or promotes the genetic mitochondrial-related disease. In some embodiments, a genetic mitochondrial-related disease comprises at least one mutation or any genetic aberration of the mitochondrial genome or in a mitochondrial gene which induces or promotes the genetic mitochondrial-related disease.

In some embodiments, mitochondrial-related disease is a metabolic disease. In some embodiments, the metabolic disease is characterized by NAD/H imbalance. In some embodiments, the metabolic disease is induced by or derived from a nutrition factor. In some embodiments, the nutrition factor comprises a food supplement. In some embodiments, the nutritional factor comprises nicotinamide or a functional analog or derivative thereof.

NRH is a precursor to NAD/H, in a newly identified salvage pathway. QR2 reduces NRH to NR, in a harmful manner that concomitantly generates the endotoxin 4-pyridone and superoxide. QR2 therefore competes with NRH kinase, as the latter utilizes NRH to salvage NAD/H. NRH generates NAD/H more potently than NR, and QR2 activity can therefore predispose the use of either one of the NAD/H precursors to affect NAD/H salvage rate/level. Critically, activating QR2 and NRH mediated NAD/H salvage pathway via addition of NRH can become toxic in oxidative phosphorylation (OXPHOS)—centric cells of the liver, while inhibition of QR2 is beneficial to liver cells and the liver. QR2 is therefore a modulator/affector of NAD/H levels that generates toxic byproducts (4-pyridone and reactive oxygen species), and which can contribute detrimentally to nicotinamide supplementation and NAD/H balance.

In some embodiments, the herein disclosed disease is for treating or preventing a liver disease. In some embodiments, a liver disease is associated with or induced by a toxin, a drug, or both. In some embodiments, a liver disease is associated with or induced by liver injury. In some embodiments, a liver disease comprises or is characterized by hepatocyte cell death. In some embodiments, hepatocyte cell death is characterized by, involves, induced by, or any combination thereof, conversion of dihydro-nicotinamide riboside (NRH) to nicotinamide riboside (NR).

In some embodiments, the treating comprises increasing the amount, abundance, levels or any combination thereof, of NRH in the subject. In some embodiments, the treating comprises increasing the amount, abundance, levels or any combination thereof, of NRH in the liver of the subject. In some embodiments, the treating comprises increasing the amount, abundance, levels or any combination thereof, of NRH in at least one hepatocyte of the subject.

In some embodiments, the treating comprises reducing the amount, abundance, levels or any combination thereof, of NR in the subject. In some embodiments, the treating comprises reducing the amount, abundance, levels or any combination thereof, of NR in the liver of the subject. In some embodiments, the treating comprises reducing the amount, abundance, levels or any combination thereof, of NR in at least one hepatocyte of the subject.

In some embodiments, the method comprises preventing or reducing inflammation of a nerve cell and/or glia cell of the subject. In some embodiments, the method comprises preventing or reducing inflammation of a nerve tissue in the subject. In some embodiments, inflammation comprises or is neuroinflammation. In some embodiments, there is provided a method for preventing or treating neuroinflammation in a subject in need thereof.

In some embodiments, the method comprises preventing or reducing apoptosis of a nerve cell and/or glia cell. In some embodiments, preventing or reducing apoptosis is of a cell derived from the subject (e.g., in vitro, such as in a plate or a tube) or in vivo (e.g., in the subject).

In some embodiments, the method comprises preventing or reducing accumulation of amyloid-beta aggregates in a subject in need thereof. In some embodiments, the amyloid-beta aggregates comprise intracellular aggregates, extracellular aggregates, aggregates within a nerve tissue, and aggregates within a nervous system or any combination thereof. In some embodiments, the intracellular and/or extracellular aggregates refer to a cell selected from a nerve cell, a glia cell, a muscle cell or any combination thereof. In some embodiments, the method comprises preventing or reducing accumulation of amyloid-beta aggregates in a nerve tissue.

In some embodiments, the method comprises preventing or reducing accumulation of tau-protein aggregates. In some embodiments, the tau-protein aggregates are within a nerve cell and/or a glia cell of the subject. In some embodiments, the tau-protein aggregates are in a nerve tissue, and/or in a nerve system of the subject.

In some embodiments, the method comprises preventing or treating frontotemporal dementia, frontotemporal lobar degeneration. In some embodiments, the method comprises preventing or reducing a tauopathy selected from: corticobasal degeneration, frontotemporal dementia, frontotemporal lobar degeneration, progressive supranuclear palsy, Pick's disease, or any combination thereof.

In some embodiments, the method comprises preventing or treating a disease or disorder associated with an abnormal accumulation of amyloid-beta aggregates, wherein the amyloid-beta aggregates are as described herein. In some embodiments, the method comprises preventing or treating a disease or disorder associated with an abnormal accumulation of tau protein. In some embodiments, the accumulation of tau protein is extracellular and/or intracellular accumulation. In some embodiments, the accumulation of tau protein is in a cell and/or a tissue of the subject.

Treatment effectiveness and identification of a subject that can benefit from a compound and/or a composition as describe herein, can be monitored/identified by methods which include MRI, PET, PET-CT, cognitive tests etc. Treatment effectiveness can be evaluated by monitoring physiological parameters and/or disease related biomarkers of the subject. Such physiological parameters are well-known in the art. Types of cognitive test suitable for determining treatment effectiveness are common and would be apparent to one of skill in the art. None limiting examples for such tests, include but are not limited to, choice test, context test, cue test, and others, such as exemplified herein.

In some embodiments, the method comprises administering the pharmaceutical composition of the invention at least 1 time, at least 2 times, at least 3 times, at least 4 times, at least 5 times, at least 7 times, or at least 10 times per day or per week or per month, or any value and range therebetween. Each possibility represents a separate embodiment of the invention. In some embodiments, the method comprises administering the composition or the combination of the invention 1-2 times per day or per week or per month, 1-3 times per day or per week or per month, 1-4 times per day or per week or per month, 1-5 times per day, 1-7 times per day or per week or per month, 2-3 times per day or per week or per month, 2-4 times per day or per week or per month, 2-5 times per day or per week or per month, 3-4 times per day or per week or per month, 3-5 times per day or per week or per month, or 5-7 times per day or per week or per month. Each possibility represents a separate embodiment of the invention.

In some embodiments, the method comprises administering the pharmaceutical composition of the invention to the subject at a daily or weekly or monthly dosage of 0.05 to 20 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 1 to 2 mg/kg, 2 to 5 mg/kg, 5 to 10 mg/kg, 10 to 15 mg/kg, 15 to 20 mg/kg including any range or value therebetween.

It should be apparent to one skilled in the art, that for example in-vitro and in-vivo assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g., decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21^{st} Ed., Lippincott Williams & Wilkins, Philadelphia, Pa., (2005).

In some embodiments, the effective amount or dose of the active ingredient can be estimated initially from in vitro assays. In one embodiment, a dose can be formulated in animal models and such information can be used to determine useful doses more accurately in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13^{th} Ed., McGraw-Hill/Education, New York, NY (2017)].

In some embodiments, the subject is afflicted with a disease or disorder associated with an abnormal QR2 expression and/or activation.

In some embodiments, the subject is afflicted with a neurological disease or disorder selected from: multiple sclerosis, Alzheimer's disease, multiple sclerosis, dementia, Parkinson's disease, Huntington's disease, Down syndrome, amyotrophic lateral sclerosis (ALS), prion disease, inflammation, infection, or any combination thereof and acquired and inherited neuropathies in the peripheral nervous system. In some embodiments, the neurological disorder is multiple sclerosis. In some embodiments, the neurological disorder is frontotemporal dementia, frontotemporal lobar degeneration and/or any other tauopathy, as described herein.

In some embodiments, preventing or treating comprises: reducing the amount, abundance, or level of reactive oxygen species (ROS), modulating autophagy, reducing inflammation, or any combination thereof, in the subject.

As used herein, the term “modulating” encompasses increasing or inhibiting/reducing.

In some embodiments, preventing or treating comprises increasing autophagy. In some embodiments, preventing or treating comprises reducing or inhibiting autophagy.

In some embodiments, reducing the amount, abundance, or level of ROS is determined in the brain or any neuronal tissue of the subject. In some embodiments, reducing the amount, abundance, or level of ROS is determined in a sample obtained or derived from the brain or any neuronal tissue of the subject.

In some embodiments, treating comprises reducing the amount, abundance, or level of ROS in the brain or any neuronal tissue of the subject.

In some embodiments, treating comprises reducing the amount, abundance, or level of at least one cytokine in the brain or any neuronal tissue, in the serum, or any combination thereof, of the subject.

In some embodiments, reducing the amount, abundance, or level of at least one cytokine is determined in a sample obtained or derived from the brain or any neuronal tissue, the serum, or any combination thereof, of the subject.

In some embodiments, the method is for reducing or inhibiting: abnormal cell proliferation, tumor growth, malignancy, or any combination thereof in a subject in need thereof. In some embodiments, the method is for reducing or inhibiting proliferation of cells expressing QR2. In some embodiments, the method is for selectively reducing or inhibiting proliferation of cells expressing QR2.

In some embodiments, reducing comprises at least 50%, at least 60%, at least 70%, at least 80%, at least 85%, at least 90%, at least 92%, at least 95%, at least 97%, at least 99% reduction of the cell proliferation, including any value there between.

In some embodiments, reducing comprises at least 50%, at least 60%, at least 70%, at least 80%, at least 85%, at least 90%, at least 92%, at least 95%, at least 97%, at least 99% reduction of QR2 activity, including any value therebetween.

In some embodiments, the compound of the invention has IC50 in inhibiting QR2 activity between 0.1 and 1 nM, between 1 and 5 nM, between 5 and 10 nM, between 10 and 50 nM, between 50 and 100 nM, between 100 and 500 nM, between 500 and 1 μM, between 1 and 5 μM, between 5 and 10 μM, including any value therebetween.

In some embodiments, the compound has at least 5 times, at least 10 times, at least 15 times, at least 20 times, at least 30 times, at least 30 times, at least 50 times, at least 80 times, at least 100 times, at least 200 times, at least 300 times, at least 400 times, at least 500 times, at least 700 times, at least 1,000 times, at least 10,000 times, at least 50,000 times, at least 100,000 times lower IC50 for QR2 as compared to other quinone reductases (e.g., QR1).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### General

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range e.g., “21-100”, is stated herein, it implies that the group, in this case the alkyl group, may contain 21 carbon atom, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine, or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “mercapto” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amino” describes a —NR′R″ group, with R′ and R″ as described herein.

The term “heterocyclyl” describes a monocyclic or fused ring group having in the ring(s) one or more atoms such as nitrogen, oxygen, and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” or “carboxylate” describes a —C(O)OR′ group, where R′ is hydrogen, alkyl, cycloalkyl, alkenyl, aryl, heteroaryl (bonded through a ring carbon) or heterocyclyl (bonded through a ring carbon) as defined herein.

The term “carbonyl” describes a —C(O)R′ group, where R′ is as defined hereinabove.

The above-terms also encompass thio-derivatives thereof (thiocarboxy and thiocarbonyl).

The term “thiocarbonyl” describes a —C(S)R′ group, where R′ is as defined hereinabove.

A “thiocarboxy” group describes a —C(S)OR′ group, where R′ is as defined herein.

A “sulfinyl” group describes an —S(O)R′ group, where R′ is as defined herein.

A “sulfonyl” or “sulfonate” group describes an —S(O)₂R′ group, where R′ is as defined herein.

A “carbamyl” or “carbamate” group describes an —OC(O)NR′R″ group, where R′ is as defined herein and R″ is as defined for R′.

A “nitro” group refers to a —NO2 group.

The term “amide” as used herein encompasses C-amide and N-amide.

The term “C-amide” describes a —C(O)NR′R″ end group or a —C(O)NR′-linking group, as these phrases are defined hereinabove, where R′ and R″ are as defined herein.

The term “N-amide” describes a —NR″C(O)R′ end group or a —NR′C(O)— linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —CN group.

The term “azo” or “diazo” describes an —N═NR′ end group or an —N═N— linking group, as these phrases are defined hereinabove, with R′ as defined hereinabove.

The term “guanidine” describes a —R′NC(N)NR″R″′ end group or a —R′NC(N) NR″— linking group, as these phrases are defined hereinabove, where R′, R″ and R″′ are as defined herein.

As used herein, the term “azide” refers to a —N3 group.

The term “sulfonamide” refers to a —S(O)₂NR′R″ group, with R′ and R″ as defined herein.

The term “phosphonyl” or “phosphonate” describes an —OP(O)—(OR′)₂ group, with R′ as defined hereinabove.

The term “phosphinyl” describes a —PR′R″ group, with R′ and R″ as defined hereinabove.

The term “alkylaryl” describes an alkyl, as defined herein, which substituted by an aryl, as described herein. An exemplary alkylaryl is benzyl.

The term “heteroaryl” describes a monocyclic (e.g., C5-C6 heteroaryl ring) or fused ring (i.e. rings which share an adjacent pair of atoms) group having in the ring(s) one or more atoms, such as, for example, nitrogen, oxygen, and sulfur and, in addition, having a completely conjugated pi-electron system. In some embodiments, the terms “heteroaryl” and “C5-C6 heteroaryl” are used herein interchangeably. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazol, pyridine, pyrrole, oxazole, indole, purine, and the like.

As used herein, the terms “halo” and “halide”, which are referred to herein interchangeably, describe an atom of a halogen, that is fluorine, chlorine, bromine, or iodine, also referred to herein as fluoride, chloride, bromide, and iodide.

The term “haloalkyl” describes an alkyl group as defined above, further substituted by one or more halide(s).

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### CRISPR Cas9 Targeting of QR2

Additional details of the knockout procedure and corresponding figures have been previously published. Disruption of the NQO2 gene at positions 46 and 108 within exon 4 was done using CRISPR/Cas9 dual nickase (plasmid pSpCas9n(BB)-2A-Puro (PX462), Addgene #48141). The target sequence was selected using a CRISPR design tool (http://crispr.mit.edu/), which identified the guide sequences with the least off-targets. Vectors NQ024_46 and NQ024_108 were produced by cloning oligonucleotides corresponding to guide RNA (sgRNA) into PX462 as previously described. The vectors were then transfected into HCT 116 cells (at 70% confluence) using Lipofectamine 2000 (Life Technologies), and 24 h later 0.7 μg/mL puromycin (Alfa Aesar) was added to the media for 72 h. Cells that survived were then collected and serially diluted into 96 well plates, with puromycin supplemented media, to enable selection and expansion of plasmid containing colonies two weeks later.

### Label-Free Mass Spectrometric Analysis

To prepare for mass spectrometry the parental C1 (HCT116^{QR+}), C3, and C5 (both HCT1161^{QR2Δ}) were seeded in 10 cm plates (n=5). Cells were cultured until reaching about 80% confluency, at which point cells were collected, washed twice with PBS, lifted from plates with trypsin-EDTA (0.25%), and centrifuged at 1,000 g; cell pellets were frozen at −80° C. Frozen cell pellets were resuspended in 8 M urea, 50 mM ammonium bicarbonate (ABC), 10 mM DTT, 2% SDS and sonicated with a probe sonicator. Twenty-five g of protein lysate, as quantified by Pierce™ 660 nm Protein Assay (ThermoFisher Scientific), was reduced in 10 mM DTT for 25 min and alkylated in 100 mM iodoacetamide for 25 min in the dark, followed by methanol precipitation as described previously. The protein pellet was resuspended in 200 μL of ABC and subjected to a sequential digest first with 250 ng of LysC (Wako Chemicals, USA) for 4 h, then 500 ng of Trypsin/LysC (Promega) for 16 h, followed by 500 ng of Trypsin (Promega) for an additional 4 h. Digestions were incubated at 37° C. with interval mixing at 600 rpm (30 s mix, 2 min pause) on a Thermomixer C (Eppendorf, catalogue #2231000667). After the last digestion, samples were acidified with 10% formic acid to pH 3 to 4 and centrifuged at 14,000 g to pellet insoluble material.

Approximately 1 μg of peptide sample was injected onto a Waters M-Class nanoAcquity HPLC system coupled to an Orbitrap Elite mass spectrometer (ThermoFisher Scientific) operating in positive mode. Buffer A consisted of mass spectrometry grade water with 0.1% formic acid and buffer B consisted of acetonitrile with 0.1% formic acid (ThermoFisher Scientific). All samples were trapped for 5 min at a flow rate of 5 mL/min using 99% buffer A and 1% buffer B on a Symmetry BEH C18 Trapping Column (5 mm, 180 mm×20 mm, Waters). Peptides were separated using a Peptide BEH C18 Column (130 A°, 1.7 mm, 75 mm×250 mm) operating at a flow rate of 300 nL/min at 35° C. (Waters). Samples were separated using a non-linear gradient consisting of 1%-7% buffer B over 1 min, 7%-23% buffer B over 179 min and 23%-35% buffer B over 60 min, before increasing to 98% buffer B and washing. MS acquisition settings are provided (See table below).

TABLE 1
Mass spectrometry acquisition settings

| Instrument | Orbitrap Elite | |
|---|---|---|
| Mass Range | 400 to 1450 | m/z |
| MS1 resolution (Orbitrap) | 120K | |
| MS1 AGC target | 10⁶ | |
| MS1 injection time | 200 | ms |
| Lock mass | 445.120025 | |
| MS2 detection | IT | |
| MS2 scan rate | rapid | |
| MS2 AGC target | 10⁴ | |
| MS2 injection time | 50 | ms |
| Top N | 20 | |
| Isolation width | 2 | |
| MS2 activation | CID | |
| Normalized collision energy | 35 | |
| Dynamic exclusion | enabled | |
| Minimum signal required | 500 | |
| Exclusion duration | 30 | s |

| | |
|---|---|
| Exclusion mass width low | 0.5 |
| Exclusion mass width high | 1.5 |
| Charge exclusion | unassigned, 1, >8 |

### Differential Expression Analysis and Gene-Set Enrichment

Differential expression analysis was performed based on LFQ intensity values calculated (see above) using Perseus (version 2.0.3.0). Missing values (proteins below detection limit) were imputed to the value of 216. Then, the two QR2 KO lines and the control were pairwise compared and tested for differential expression using a non-parametric different variance adjusted t-test. A permutational false discovery rate (FDR) procedure was done to account for false positive discovery. For FDR adjusted q value <0.05 no significant difference was found between the two lines. The differentially expressed (DE) proteins included 317 proteins and 294 proteins for QR2 KO lines, C₃ and C₅, respectively. Of these, 182 proteins were DE in both lines. In order to compare the effect of QR2 CRISPRi between the two lines, Pearson correlation between QR2 CRISPRi vs. control log 2 fold change values were calculated and tested (Pearson r=0.83, P=2.2e⁻¹⁶). Since no significant differences were found between lines, and high Pearson correlation was found, the inventors continued for gene-set enrichment analyses using a combined set of DE proteins from both lines. This set included 429 proteins in total, 258 with higher expression and 171 with lower expression in QR2Δ lines compared to control. In order to predict association between proteins among higher and among lower expressed proteins, a STRING association network was calculated, with confidence parameter set to 0.8 (high confidence), omitting ‘text mining’ from the active interaction sources options and retaining only connected proteins in the network. The inventors examined the overlap between the present set of 429 DE proteins and the list of proteins DE in DLPFC tissues of Alzheimer's disease compared to control reported in Johnson et al., (2020). The latter list included 955 proteins (474 higher expressed and 478 lower expressed in AD compared to control tissues). Genes overlapping between the two lists and in opposite direction (QR2Δ down-AD up and vice versa) were examined for functional enrichment using ENRICHR inspecting results from BioPlanet, KEGG and Gene Ontology databases. Functional enrichment was considered significant for FDR adjusted P value <0.05. For selected pathways with significant enrichment in the overlapping gene-set, the inventors also verified enrichment in the dataset from the two studies separately. This was done using ENRICHR with the same criteria described above. Z-score for enrichment was calculated as (up−down)/\sqrt{N} where up and down and the number of genes DE in both direction, and N is the total number of DE genes.

### Western Blot

Samples in SDS sample buffer were loaded into 10-12.5% polyacrylamide gels, using equal protein quantities, and subjected to SDS-PAGE. Following electrophoresis, samples were transferred onto 0.2 μm nitrocellulose membranes (Bio-Rad) using Trans-Blot Turbo Transfer System (Bio-Rad), washed three times in TBST, blocked for 1 h in blocking buffer (Bio-Rad) and incubated overnight at 4° C. with primary antibodies, including Tubulin (1:40,000, Sigma, SAB4500087), QR2 (1:1,000, Santa Cruz, sc-271665), NDUFA9 (1:500, AbCam, ab14713) and CD73 (1:1,000, Cell-Signaling, D7F9A) diluted in blocking buffer. The next day, the membranes were washed three times in TBST and incubated at room temperature (RT) for 1 h with 1:10,000 secondary antibodies conjugated to horseradish peroxidase, and following a further three times washes in TBST were immunoblotted with Westar Supernova (Cyanagen), imaged using a charge-coupled device camera and analyzed using Quantity One software (Bio-Rad).

### H₂DCFDA Detection of Cellular ROS

The cells were harvested and resuspended in 1% clear medium (DMEM—with no phenol red, supplemented with 1% FBS, 1% L-Glutamine and 1% Penicillin-Streptomycin). A sterile poly-L-lysine-coated Nunc black, 96-well, clear flat-bottomed plate (Thermo Fisher Scientific) was seeded with 25,000 cells in 100 μL per well and incubated overnight. The next day, the medium was removed, and the wells carefully washed with PBS. H₂DCFDA (20 μM, Sigma-Aldrich) was added in a total volume of 100 μL to each well (a control group contained only PBS). The plate was then incubated in the dark at 37° C. for 45 min. The H₂DCFDA solution was removed, and the wells were washed with PBS. After the PBS was removed, the treatments (100 μL per well) were applied, using H₂O₂ (2 mM) for applied stress. The plate was then incubated in the dark at 37° C. for 3 h, before reading in a Tecan M200 Pro florescence microplate reader, using wavelengths Ex/Em=485 nm/535 nm.

### High Throughput Screen (HTS)

The enzymatic activity of QR2 (10 nM) was measured using menadione (both Sigma-Aldrich) as substrate and dihydrobenzylnicotamide (BNAH; Tocris) as co-substrate (Boutin et al., 2005). The reaction was performed in 50 mM Tris HCl, 150 mM NaCl 0.01% Tween-20 pH=8.5 (Sigma-Aldrich) at room temperature. Briefly, 3 μL of menadione alone (200 μM) or a mixture of menadione and QR2 (200 μM of menadione and 10 nM of QR2) were dispensed in 1536-well black Nunc plates (Thermo-Fisher) using a Multidrop™ Combi Reagent Dispenser (Thermo-Fisher) and pre-incubated with compounds for 10 min. BNAH (200 μM) was then added (3 μL) to the reaction using a BioNex Solutions BNX 1536 plate washer and the plates were incubated for another 10 min. Fluorescence intensity was followed in a Pherastar FS multi-mode plate reader (BMG Labtech) using an optic module with excitation at 360 nm and emission at 470 nm. The initial HTS was done with approximately 200,000 compounds at a single concentration of 5 μM. The chemical libraries used were Selleck Chemicals Bioactives, the Drug-like set from Enamine, HitFinder collection from Maybridge, Spectrum Collection from Microsource, Lopac from Sigma-Aldrich and the diversity sets from ChemDiv and ChemBridge. Initial “hit” compounds were defined as causing >30% reduction of enzyme activity. Compounds suspected to be fluorescent were re-assayed in an absorbance assay at 350 nm using the same reaction but in a higher volume (100 μL), in a transparent plate (Greiner Bio-One). “Hit” compounds resulting from both the fluorescence and absorbance assay were tested in a dose-response, and median inhibition concentration (IC50) values were determined. In parallel, the same dose response was used in a selectivity assay where QR2 enzyme was replaced by QR1 (also known as—NQO1, 10 nM; Sigma-Aldrich).

### Synthesis of Novel QR2 Inhibitors

### General

All reagents and solvents used for the synthesis were purchased from Sigma-Aldrich, Merck and Acros. Chemical building blocks were purchased from Enamine, Combi-Blocks and MolPort chemical Suppliers. Commercial reagents were used for synthesis without further purification. All solvents used for flash chromatography were HPLC grade. Reactions on microwave were done on Microwave reactor: Biotage Initiator+. Flash chromatography was performed using Merck Silica gel Kieselgel 60 (0.04-0.06 mm) or by atomized CombiFlash® Systems (Teledyne ESCO, USA) with RediSep Rf Normal-phase Flash Columns. Purification of the final compounds was performed using preparative HPLC; Waters Prep 2545 Preparative Chromatography System, with UV/Vis detector 2489, using XBridge® Prep C18 10 μm 10×250 mm Column (PN: 186003891, SN:161I3608512502). Reaction progress and compounds' purity was monitored by Waters UPLC-MS system: Acquity UPLC® H class with PDA detector and using Acquity UPLC® BEH C18 1.7 μm 2.1×50 mm Column (PN:186002350, SN 02703533825836). MS-system: Waters, SQ detector 2. ¹H and ¹³C NMR spectra were recorded on a Bruker Avance III −300 MHz, 400 MHz and 500 MHz spectrometer, equipped with QNP probe. Chemical shifts are reported in ppm on the δ scale and are calibrated according to the deuterated solvents. All J values are given in Hertz.

### General Synthetic Procedure of the Exemplary Compounds of the Invention

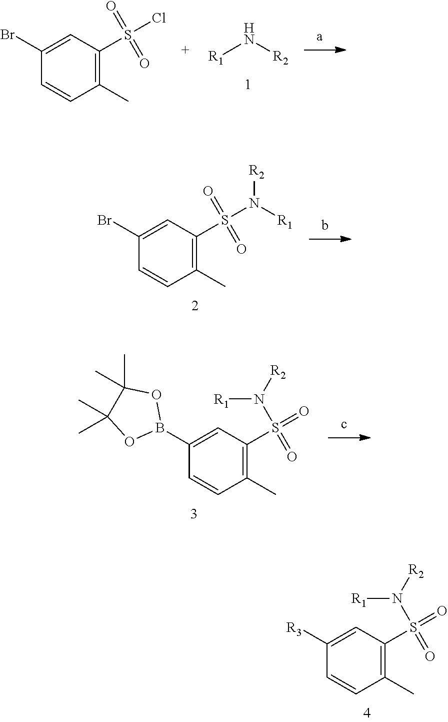

Reaction condition: (a) 5-bromo-2-methylbenzenesulfonyl chloride, DMAP, pyridine, desired amine (1), DCM, 0° C., 30 min; (b) 5-bromo-2-methyl-N-benzenesulfonamide (2), Potassium acetate, bis(pinacolato)diboron, PdCl₂(dppf), 1,4-dioxane, −80° C., 12 h; (c) desired ((N-sulfamoyl)-4-methylphenyl)boronic ester (3), desired heterocycle with leaving group (R3-Br/OTf), potassium carbonate, PdCl₂(dppf), 1,4-dioxane, 90° C., 4-12 h.

### 5-bromo-2-methyl-N-benzenesulfonamides (2)

5-bromo-2-methylbenzenesulfonyl chloride (1 eq.), DMAP (0.1 eq.) and pyridine (1.5 eq.) were added to a cooled round bottom flask with dichloromethane (0.2 M). The desired amine (1.4 eq.) was slowly added dropwise, and the mixture was stirred for 30 min. Reaction was monitored by liquid chromatography-mass spectrometry (LC-MS), after the completion of the reaction the crude washed with water, the organic layer washed with NaHCO₃ solution and brine, and then dried over Na₂SO₄. The crude product was purified on ESCO CombiFlash System using a silica gel column 12 g, dichloromethane (DCM)+1%, methanol (MeOH) to ethyl acetate (EA)+1% MeOH gradient 25 min.

### ((N-sulfamoyl)-4-methylphenyl)boronic acids (3)

Potassium acetate (3 eq.), 5-bromo-2-methyl-N-benzenesulfonamide (1 eq.) and bis(pinacolato)diboron (1.2 eq.) were added to a microwave vial and dissolved in 1,4-dioxane (0.15 M). After degassing with Ar for 15 min, PdCl₂(dppf) (0.05 eq.) was added, and the reaction mixture was heated in a microwave at 80° C. for 12 h. The reaction was monitored by LC-MS. Reaction mixture was cooled to room temperature and filtered through celite. The solvents were evaporated, and the crude product used as is in the next stage.

### N-2-methyl-5-benzenesulfonamide (4)

The desired ((N-sulfamoyl)-4-methylphenyl)boronic ester (crude) (1 eq.), the desired heterocycle with leaving group (1.5 eq.), and potassium carbonate (3 eq.) were dissolved in 1,4-dioxane:water 2.5:1 (90 mM). The reaction mixture degassed for 15 min with argon, then PdCl₂(dppf) (0.05 eq.) was added. The vial was heated in a sand bath to 90° C., for 4-12 h. The reaction was monitored by LC-MS. The reaction mixture was cooled to room temperature and filtered through celite and washed several times with EtOAc. The solvents were evaporated, and the crude product was purified on ESCO CombiFlash System using a silica gel column 12 g, DCM+1% MeOH to EA+1% MeOH gradient 25 min. Some of the final products were then subjected to preparative HPLC; Waters Prep 2545 Preparative Chromatography System, the solvents were evaporated to yield a range of white/off-white solids.

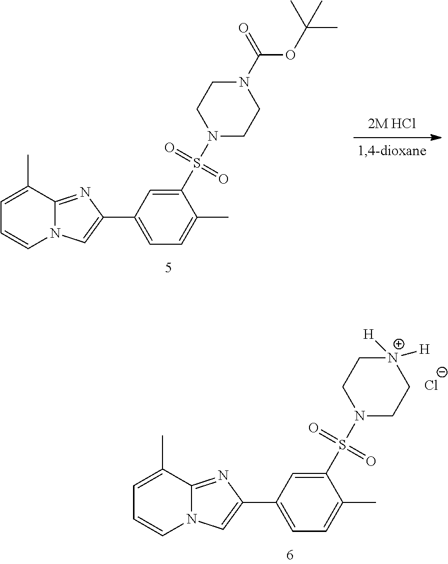 [SMILES: Cc1ccc(-c2cn3cccc(C)c3n2)cc1S(=O)(=O)N1CCN(C(=O)OC(C)(C)C)CC1 ; Cc1ccc(-c2cn3cccc(C)c3n2)cc1S(=O)(=O)N1CC[NH2+]CC1 ; [Cl-]]

### 4-((2-methyl-5-(8-methylimidazo[1,2-a]pyridin-2-yl)phenyl)sulfonyl)piperazin-1-ium (6)

tert-butyl 4-((2-methyl-5-(8-methylimidazo[1,2-a]pyridin-2-yl)phenyl)sulfonyl)piperazine-1-carboxylate (5) (3.00 g, 6.37 mmol) was dissolved in 15 mL 1,4-dioxane then added HCl 4 M in 1,4-dioxane (15 mL) and stirred for 6 h, then an additional HCl 4 M in 1,4-dioxane (5 mL) and stirred 12 h. The reaction was then heated to 60° C. for 4 h and cooled to 0° C., and the reaction was diluted with 50 mL ether, and filtered of the white solid which was dried overnight under high vacuum to give 8-methyl-2-(4-methyl-3-(piperazin-1-ylsulfonyl)phenyl)imidazo[1,2-a]pyridine hydrochloride (6) with a 77.5% yield.

### Cell Lines

Cell lines were grown at 37° C. with 5% CO2. HEK293T and HCT116 cells were cultured in DMEM (Gibco) supplemented with 10% FBS (Biological Industries), 100 U/mL penicillin/streptomycin (Biological Industries) and 2 mM of L-Glutamine (Biological Industries). Cells were grown in 10 mm plates until 80-90% confluency and passaged every 3-7 days at dilution ratios ranging from 1:5 to 1:30, depending on the initial density. The number of total passages did not exceed more than 5 in HCT116, or 20 in HEK293T, prior to the start of an experiment. THLE-2 (CRL-2706™) were grown in the BEGM Bullet Kit (CC-3170) from Lonza. Besides the additives contained in the kit, the medium was further supplemented with 5 ng/mL EGF (Sigma), 70 ng/mL phosphoethanolamine (Sigma) and 10% FBS (Biological Industries). The plates for the THLE-2 needed to be pre-coated with a mixture of 0.01 mg/mL fibronectin, 0.05 mg/mL of PureCol™ EZ Gel Solution (Sigma) and 0.01 mg/mL of BSA dissolved in BEBM medium (Lonza). The coating medium was aspirated before seeding.

### Cell Toxicity Assay

THLE-2 cells were exposed to the compounds in a 9-point, 2-fold dilution dose response series with a 100 μM as upper limit, for 72 h. Following the 72 h of exposure to the test compounds, cell viability was determined by measuring the concentration of cellular ATP (CellTiter Glo, Promega). The luminescence signal was measured on a Pherastar FS multi-mode plate reader (BMG Labtech). Each data point was tested in a triplicate.

### XTT Cell Viability Assay

HEK293FT cells were cultured with penicillin-streptomycin antibiotics in Invitrogen DMEM (Thermo Fisher Scientific) with 10% fetal bovine serum and L-glutamine and grown to 80-90% confluence in 100 mm plates, while being passaged every 2-3 days. For the XTT assay (Biological Industries), cells were then collected, centrifuged, and resuspended in fresh minimum essential medium, and seeded at 50,000 cells/well in 96-well, poly-L-lysine-coated Nunc black, clear flat-bottomed plates (Thermo Fisher Scientific). The next day, the cells were treated with increasing doses of QR2 inhibitors or vehicle for 3 h, and the XTT assay was then carried out as according to the manufacturer's instructions.

### Cellular Thermal Shift Assay (CETSA)

The CETSA assay was performed as described (Miettinen and Björklund, 2014) with some modifications. Briefly, HEK293T cells were trypsinized (Biological Industries), washed in PBS (Sigma-Aldrich), and then suspended in PBS containing protease inhibitor cocktail (Roche). The suspended cells were then divided into two Eppendorf tubes and were treated with either: a compound or DMSO (Sigma-Aldrich) for 1 h at 37° C. under shaking. Following treatment, each sample was divided into PCR tubes (100 μL/tube) and subjected to a temperature gradient (ranging from 63 to 80° C.) for 3 minutes. Cell lysates were obtained by 3-cycles of freeze-thaw using liquid nitrogen and a thermal block set to 25° C. Samples were then centrifuged at 15,000 rpm at 4° C. for 20 min and were subsequently analyzed by western blot targeting QR2 (1:100, Santa Cruz; sc-271665). The isothermal dose response fingerprint (ITDRF) experiments were done using a constant temperature of 73° C. Band intensities were normalized to the highest concentration and superoxide dismutase 1 (SOD1) levels (1:200, Santa Cruz; sc-17767). Analysis of the results were performed according to Jafari et al., (2014) using GraphPad Prism software.

### Cloning, Expression & Purification of QR2

Full length QR2 (1-232) with an N-terminal Avi-tag was cloned into the expression vector pET28-bdSumo. This vector was constructed by transferring the His14-bdSUMO cassette from the expression vector (designated K151) generously obtained from Prof. Dirk Gorlich from the Max-Planck-Institute, Gottingen, Germany into the expression vector pET28-TevH. Cloning was performed by the Restriction-Free (RF) method. The plasmid was co-transformed with pACYC184-BirA into BL21 (DE3). A 5 L culture was induced with 200 mM IPTG and grown at 15° C. ON. The culture was harvested and lysed by a cooled cell disrupter (Constant Systems) in lysis buffer (50 mM Tris pH 8, 0.5 M NaCl, 20 mM Imidazole) containing 200 KU/100 ml lysozyme, 20 μg/ml DNase, 1 mM MgCl₂, 1 mM phenylmethylsulphonyl fluoride (PMSF) and protease inhibitor cocktail. After clarification of the soup by centrifugation, the lysate was incubated with 5 ml washed Ni beads (Adar Biotech, Israel) for 1 h at 4° C. After removing the soup, the beads were washed 4 times with 50 ml lysis buffer. Avi-tag-QR2 eluted by incubation of the beads with 5 ml cleavage buffer (50 mM Tris pH 8, 0.5 M NaCl and 0.4 mg bdSumo protease) for 2 h at RT. The supernatant containing the cleaved Avi-tag-RQ2 was removed, and an additional 5 ml cleavage buffer was added to the beads for 2 h at RT. The two elution solutions were combined, concentrated and applied to a size exclusion (SEC) column (HiLoad_16/60_Superdex200 prep-grade, GE Healthcare) equilibrated with 50 mM Tris 8.5, 150 mM NaCl. The pure biotinylated Avi-tag-QR2 which migrates as a single peak at 85 ml, was pooled and flash frozen in aliquots using liquid nitrogen and was stored at −80° C.

Crystallization, Data Collection and Refinement of hQR2

Purified hQR2 was co-crystallized in the presence of FAD and YB-537, using the hanging drop vapor diffusion method and a Mosquito robot (TTP LabTech) at 19° C. The hQR2 crystals grew utilizing the precipitants 0.7 M ammonium tartrate dibasic and 50 mM Tris pH 8.5 and formed in the orthorhombic space group P2₁2₁2₁, with one dimer per asymmetric unit. Data to 2.25 Å resolution was collected in-house, using a Rigaku RU-H3R X-ray instrument. All diffraction images were indexed and integrated using the XIA2 program (Evans and Murshudov, 2013), and the integrated reflections were scaled using the SCALA program (Evans, 2006). Structure factor amplitudes were calculated using TRUNCATE (French and Wilson, 1978) from the CCP4 program suite. The structure of hQR2 was solved by molecular replacement with the program PHASER (McCoy, 2006), using the hQR2 in complex with CL097 (PDB-TD code 5LBU). All steps of the atomic refinements were performed with the PHENIX.refine, Parallel PHENIX.phaser programs (Afonine et al., 2012). The model was built into 2mFobs-DFcalc, and mFobs-DFcalc maps using COOT (Emsley and Cowtan, 2004). The model was optimized using PDB_REDO (Joosten et al., 2011), and was evaluated with MOLPROBIDITY (Chen et al., 2010). Electron density revealed unambiguous density for the bound FAD and YB-537. Details of the data collection and refinement statistics of the hQR2 structure are described in Table 2.

TABLE 2
Data collection and refinement statistics for hQR2
Data Collection

| Space group | P2₁2₁2₁ | |
|---|---|---|
| Cell dimensions: | | |
| a, b, c (Å) | 57.03 83.36 106.49 | |
| α, β, γ (°) | 90, 90, 90 | |
| No. of copies in a.u. | 2 | |
| Resolution (Å) | 47.07-2.25 | |
| Upper resolution shell (Å) | 2.33-2.25 | |
| Unique reflections | 24,796 | (2,419) |
| Completeness (%) | 99.90 | (99.38) |
| Multiplicity | 10.2 | (10.2) |
| Average I/σ(I) | 8.82 | (3.51) |
| R-pim | 0.06999 | (0.2235) |
| CC1/2 | 0.993 | (0.902) |

| | |
|---|---|
| Refinement | |
| Resolution range (Å) | 47.07-2.25 |
| No. of reflections (I/σ(I) > 0) | 24,777 |
| No. of reflections in test set | 2419 |
| R-working/R-free | 0.1748/0.2256 |
| No. of protein atoms | 3599 |
| No. of water molecules | 116 |
| Overall average B factor (Å²) | 19.24 |
| Root mean square deviations: | |
| bond length (Å) | 0.011 |
| bond angle (°) | 1.54 |
| Ramachandran Plot | |
| Most favored (%) | 96.71 |
| Additionally allowed (%) | 2.85 |
| Disallowed (%) | 0.44 |

* Values in parentheses refer to the data of the corresponding upper resolution shell

### Subjects

Male Sprague Dawley rats 225-400 g and 8 week-old, and 20-35 g C57BL/6 (Envigo) male mice were used. All animals were housed in the University of Haifa core facilities, in a temperature controlled environment (22-24° C.), on a 12 h light/12 h dark cycle (light phase 07:00-19:00), with food and water provided ad libitum. All cages were enriched with cotton wool bedding and sections of piping, to provide additional hiding and nesting areas within the cage. All experiments were approved by the University of Haifa Animal Care and Use committee (license numbers 437, 488, 631, 635, 642). Animals were given 7 days of acclimatization before experimentation, and during the entire period, animals were handled in accordance with University of Haifa practices and standards, in compliance with the National Institutes of Health guidelines for the ethical treatment of animals.

### Blinding Measures for Animal Experiments

For double blind experiments with animals, one experimenter prepared inhibitors and vehicle (both completely translucent liquids resembling water). Both vehicle and inhibitors were made using the same volume in identical vessels, once a week, and labelled with a code (e.g.,—bottle AY, bottle XZ). A second experimenter then received bottles A and B, blind to their identity, and moved their contents into bottles with a different code, to which the first experimenter was blind (e.g.,—bottle 1, bottle 2). Finally, another experimenter/s blind to the identity of either codes was/were given bottles AY and XZ, gave the animals the inhibitors and vehicle and carried out the behavioral experiments and analysis. Once data was collected and analyzed, the animal and treatment identity was revealed. Subsequent immunohistochemistry image analysis was done by another experimenter, blind and unaware of any of the groups, treatments, sex or other identities of the subjects.

### Microinjections

### Rats

Anesthesia was performed with 0.3 ml/100 g body weight equithesin (2.12% MgSO₄, 10% ethanol, 39.1% 1,2-propanolol, 0.98% sodium pentobarbital, and 4.2% chloral hydrate (Sigma-Aldrich). Using a stereotaxic device (Stoelting), 10 mm, 23-gauge steel guide cannulas were bilaterally installed over the anterior insular cortex (aIC), according to the coordinates (with reference to bregma): AP 1.2 mm, ML±5.5 mm, DV 5.5 mm (Paxinos & Watson, 2006). Acrylic dental cement was applied to the cannulas as well as over two anchoring screws fastened to the skull, in order to fix the cannulas in place. A 7-day period of recovery was then provided to the rats, during which they received antibiotics (0.5 mg/kg of Baytril©, enrofloxacin) and analgesic treatment (0.5 mg/kg norocarp) for the 3 days following the surgery. In order to infuse QR2 inhibitor YB-808 20 min prior to taste learning, the guide cannula stylus was removed, and a 28-gauge injection cannula inserted, up to 0.5 mm beyond the end of the guide cannula. The injection cannula was fitted with PE20 tubing to a Hamilton microsyringe and 1 μL of vehicle (0.002% DMSO) or YB-808 (20 μM) was delivered at 0.5 L/min. In order to prevent withdrawal of the injected content from the injection site, cannulas were kept in place for an additional minute prior to removal.

### Mice

Mice were anesthetized under 2% isoflurane, using an induction box (HME109, Highland Medical Equipment). They were placed in a stereotaxic device (Kopf Stereotaxic Alignment System, model 1900) under continuous 1% isoflurane anesthesia. Guide cannulas were implanted bilaterally to CA1 (from bregma: −1.9 mm AP, ±1.4 mm ML, −1.6 mm DV), cemented to the skull and fitted with 28-gauge dummy cannulas extending 0.2 mm beyond the tip of the 1.2 mm guide cannulas. The mice were allowed at least 7 days of recovery before experimentation. The QR2 inhibitor YB-808 was dissolved in DMSO and further diluted in saline to a final DMSO concentration of 0.1%. YB-537 was dissolved in saline (0.9%). A total of 1 μl of 5 μM of either compound or vehicle was infused bilaterally to CA1, via a 28-gauge infusion cannula projecting 0.4 mm (drug delivery depth bregma: −1.6 mm DV) beyond the guide cannula, connected by polyethylene tubing to a 10 μl syringe (Hamilton) over the course of 1 min. The injection cannula was kept for 60 s inside the guide cannula in order prevent osmotic seepage of the doses upward through the cannula tract. Twenty minutes following the injection, animals underwent delay fear conditioning. Following experimentation, animals were sacrificed, brains were excised and sectioned in coronal sections, and cannula implantation was validated by imaging.

### Pharmacokinetic Study

Pharmacokinetic (PK) and acute toxicity analysis following intravenous (i.v.) 10 mg/kg or per os (p.o.) 50 mg/kg in C57BL/6J mice was carried out by Enamine Biology Services.

### Behavior

### Incidental Taste Learning

As previously described, rats were taught to drink from two pipettes filled with 10 mL water during a 20 min period, over the course of 3 days. On the fourth day, the pipettes were filled with NaCl (0.3%), whereupon the rats experience and incidentally learn the novel taste. On the fifth day, rats were once again given water in the pipettes. On the sixth day, the rats were presented with a choice test, in which they are given two pipettes of water and two pipettes of NaCl (10 mL, each). The memory for the novel taste is then assessed following 20 min of liquid consumption, by calculating a preference index thus: [novel taste consumed/(novel taste consumed+water consumed)]×100.

### Delay Fear Conditioning

Mice are transported to the conditioning room, which is lit by red light only, and kept there for 2 min. They were then placed inside a Habitest Operant Cage, within a Habitest Isolation Cubicle (Coulbourn), on a modular shock floor made of 16 metal grids, connected to Precision Animal Shockers (Coulbourn) with illumination inside the cage coming from a 20 W bulb. The mice were given 2 min to explore, during which baseline freezing was measured. Then, a 20 s, 4 kHz, 80 dB tone was given, co-terminating with the start of a 2 s, 0.5 mA foot-shock, which was repeated a further two times, each having 1 min interval. Following the third, and last of these bouts, 1 min was given prior to the animals being removed from the chambers. The next day, the animals were returned to the room under the same conditions, and were placed back into the chambers, and freezing to the context was measured over the course of 5 min. The next day, the mice were once again returned to the conditioning room, except the room was lit with white light, while the chambers were darkened, the chamber floor was covered with a flat, smooth plastic cover, one of the walls was fitted with a paper sticker and the chamber was scented with diluted (10%) window cleaner (Sano). In this unfamiliar context, the protocol from the first day was repeated, except without the foot-shocks. Freezing for the cue was recorded during the tone. All measurements were taken with a Sentec stc-tb33usb-at camera, and analysis was performed with Freeze Frame software (Actimetrics).

### Morris Water Maze

Morris water maze (MWM) was carried out as previously described (Vorhees et al., 2006). At the same time each consecutive day, mice were placed in the dimly lit room containing the maze for 10 min, within cages containing dry bedding (not the home cages). The maze was obscured by a non-light-permeable curtain. The maze consisted of a 1.2 m pool, with RT (21-23° C.) water colored light grey, to hide the submerged (under 1-2 cm) transparent, 10 cm in diameter plastic escape platform, which was placed at the south-western quadrant of the pool. Mice were trained 4 times a day, using 60 s trials every 30 min during which they were placed into the pool, each time from a different quadrant, and allowed to swim and find the escape platform. Upon reaching the platform, mice were removed from the pool. If the mice failed to find the pool within 60 s, they were carefully placed on the escape platform and held there for 15 s prior to being taken out of the pool. All trials were filmed with a video tracking system using EthoVision 14 (Noldus Information Technology), and escape latency (time to find the submerged escape platform) was determined by manual video analysis (due to automated detection settings unable to discern all mice coat colors—black, white and brown—against the opaque water).

### Open Field

Mice were individually placed within a cage and taken to a dimly lit room containing an open field arena 50×50 cm in size. They were given 10 min prior to being placed within the arena, where for a period of 5 min they were allowed to explore. Two weeks later, this was repeated. The mice were filmed with an Ikegami ICD-49E camera with EthoVision 14 (Noldus Information Technology). The floor was either white or black, depending on mouse coat color, to allow automatic analysis of movement parameters, apart from rearing which was manually counted.

### Novel Object Recognition

Following the first exploration of the open field arena (described above), mice were returned to the same arena the next day, while the arena now contained two identical objects. Mice were allowed to explore the objects and the arena 3 times for 10 min, with an inter trial interval of 10 min. The following day one of the objects was replaced with a novel object, and the mice were returned to the arena and allowed to explore for 10 min. Mouse movement, exploration and nuzzling was automatically recorded with an Ikegami ICD-49E camera with EthoVision 14 (Noldus Information Technology). Discrimination of the novel object was assessed by calculating (time exploring novel object−time exploring familiar object)/(time exploring novel object+time exploring familiar object).

### Nesting

Each home cage was given six identical portions of cotton wool tubes and 24 h later the nest made was photographed from above. The images were given the names of the respective cages, which were coded (see ‘Blinding Measures for Animal Experiments’ section) and were analyzed by an experimenter that was blind to the conditions, groups and cage codes. Scores were given at a scale of 1 to 5, as previously reported.

### Immunohistochemistry

A week after the completion of all behavioral experiments, 5×FAD mice were anesthetized with isoflurane, and once fully anesthetized, were transcardially perfused with 4% paraformaldehyde (PFA), dilutes in 0.1% phosphate buffered saline (PBS, Sigma-Aldrich). Brains were then briefly removed and placed in chilled, 4% PFA for 48 h, followed by immersion in 30% sucrose in 0.1 M PBS for a further 48 h. Brains were then stored at −80° C., until they were sliced into 40 μm coronal sections using a Leica CM 1950 cryostat. Slices were then washed ×3 in PBS and blocked for 1 h at RT using 10% normal donkey serum (DNS) and 0.2% triton (Sigma-Aldrich) in PBS. Antibodies, including 4-HNE (1:500, AbCam, ab48506), Iba1 (1:2,000, AbCam, ab5076), phospho-tau (1:1,000, Thermo, MN1020) and amyloid R (1:1,000, AbCam, ab201060) diluted in PBS with 10% DNS were incubated at 4° C. overnight. The next day, the slices were washed ×3 times in PBS, and secondary antibodies including donkey anti goat Alexa Fluor 568 (AbCam, ab175704), donkey anti-mouse Cy 5 (Jackson Immuno Research, 715-175-151) and donkey anti-rabbit DyLight 488 (AbCam, ab98488) all diluted 1:500 in PBS with 1% BSA, were applied to the slices at RT for 2 h. Following the incubation, the slices were washed ×3 times in PBS, mounted onto glass slides, were uniformly covered in DAPI containing Vectashield (H-1200) and coverslips were added. Images were then taken using a confocal microscope (Olympus IX83), with identical acquisition parameters across every sample per antibody. Three slices were used per mouse per antibody, with images taken of dorsal CA1 (Bregma: −1.58 mm to −2.30 mm; with each antibody having a slice from anterior, medial and posterior regions of this range) using a ×20 objective, acquiring two 800×800 pixel images over as many Z levels possible per slice (Z-stack of the whole section). Analysis of the images was done blind, using Imaris (Bitplane) software. Surface reconstruction module was used to extract the data as volumes and signal intensities. Marker volume or intensity were normalized to the corresponding brain volume. This was done in order to normalize the marker volumes to variations in Z-stack volumes and/or changes to shape and size that needed to be performed during analysis due to debris, vasculature, corpus callosum elimination, or any other obstructions or changes in shape. The normalized value was averaged for each triplicate.

### Statistical Analysis

Experimental grouping was randomly allocated, in both rats and mice. The size for each group was based on previously published results by means of similar methods, with the use of an online power calculator (https://www.stat.ubc.ca/-rollin/stats/ssize/n2.html). Shapiro-Wilk normality tests were done for the collected data. Analysis of normally distributed data was done using parametric tests (i.e.,—unpaired students t-test, one-way ANOVA followed by Tukey's or Sidak post hoc analysis) and for data not normally distributed, non-parametric tests (e.g.,—Mann-Whitney tests or Kruskal-Wallis followed by Dunn's multiple comparisons tests). Data are presented as means with SEM. All statistical analysis were done using GraphPad Prism 7 software, unless stated otherwise.

TABLE 3
Complete list of sulfonamide HTS hits and newly synthesized QR2 inhibitors

| | | QR2 | | QR1 | | |
|---|---|---|---|---|---|---|
| | | IC50 (μM) | | IC50 (μM) | | THLE2 |
| | | Absor- | Fluor- | Absor- | | IC50 |
| Compound | Structure | bance | escence | bance | Fluorescence | (μM) |
| PCM-0119249*^{/&} | 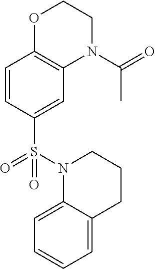 [SMILES: CC(=O)N1CCOc2ccc(S(=O)(=O)N3CCCc4ccccc43)cc21] | >9.99 | >9.90 | 3.48 | 4.7 | — |
| PCM-0119438*^{/&} | 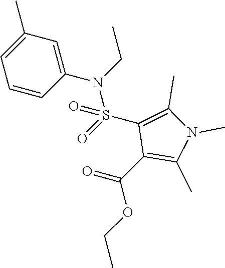 [SMILES: CCOC(=O)c1c(S(=O)(=O)N(CC)c2cccc(C)c2)c(C)n(C)c1C] | 8.98 | 3.59 | >9.99 | 5.39 | — |
| PCM-0119478*^{/&} | 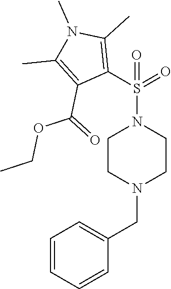 [SMILES: CCOC(=O)c1c(S(=O)(=O)N2CCN(Cc3ccccc3)CC2)c(C)n(C)c1C] | >9.99 | >9.90 | >9.99 | >9.90 | — |
| PCM-0120203*^{/&} | 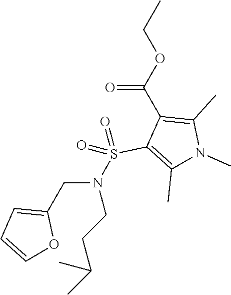 [SMILES: CCOC(=O)c1c(S(=O)(=O)N(CCC(C)C)Cc2ccco2)c(C)n(C)c1C] | 8.53 | 8.21 | >9.90 | 3.79 | — |
| PCM-0120247*^{/&} | 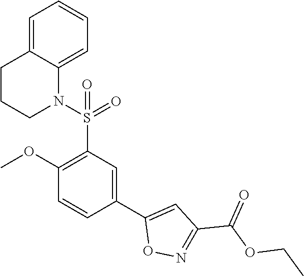 [SMILES: CCOC(=O)c1cc(-c2ccc(OC)c(S(=O)(=O)N3CCCc4ccccc43)c2)on1] | >9.99 | >9.90 | >9.99 | >9.90 | — |
| PCM-0121306*^{/&} | 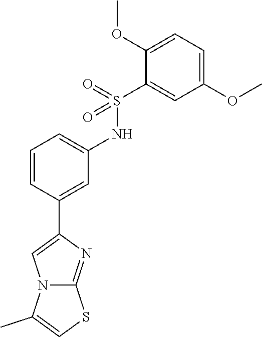 | 2.03 | 0.83 | >9.99 | 8.77 | — |
| PCM-0124575*^{/&} | 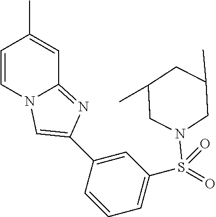 [SMILES: Cc1ccn2cc(-c3cccc(S(=O)(=O)N4CC(C)CC(C)C4)c3)nc2c1] | 1.22 | 2.18 | >9.99 | >9.90 | — |
| PCM-0124615*^{/&} | 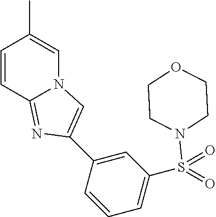 [SMILES: Cc1ccc2nc(-c3cccc(S(=O)(=O)N4CCOCC4)c3)cn2c1] | 1.65 | 3.25 | >9.99 | >9.90 | — |
| PCM-0124655*^{/&} | 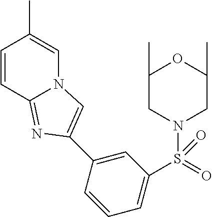 [SMILES: Cc1ccc2nc(-c3cccc(S(=O)(=O)N4CC(C)OC(C)C4)c3)cn2c1] | 3.03 | 4.42 | >9.99 | >9.90 | — |
| PCM-0124772*^{/&} | 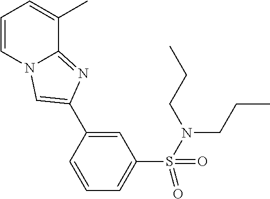 [SMILES: CCCN(CCC)S(=O)(=O)c1cccc(-c2cn3cccc(C)c3n2)c1] | 0.75 | 0.83 | >9.99 | >9.90 | — |
| PCM-0127546*^{/&} | 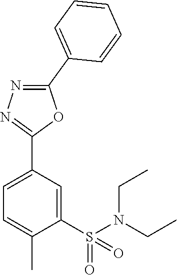 [SMILES: CCN(CC)S(=O)(=O)c1cc(-c2nnc(-c3ccccc3)o2)ccc1C] | 3.12 | 4.82 | >9.99 | >9.90 | — |
| PCM-0128885*^{/&} | 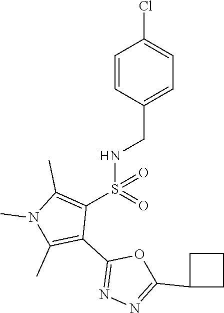 [SMILES: Cc1c(-c2nnc(C3CCC3)o2)c(S(=O)(=O)NCc2ccc(Cl)cc2)c(C)n1C] | >9.99 | >9.90 | >9.99 | >9.90 | — |
| PCM-0131156*^{/&} | 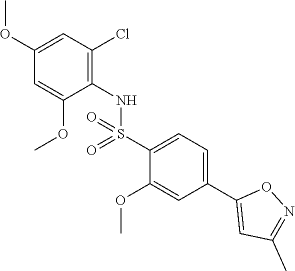 [SMILES: COc1cc(Cl)c(NS(=O)(=O)c2ccc(-c3cc(C)no3)cc2OC)c(OC)c1] | 2.73 | 3.82 | >9.99 | >9.90 | — |
| PCM-0136246*^{/&} | 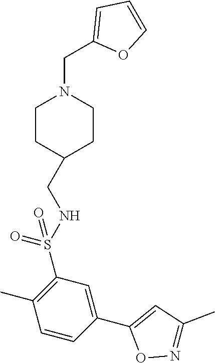 | 2.56 | 3.98 | >9.99 | >9.90 | — |
| PCM-0154188*^{/&} | 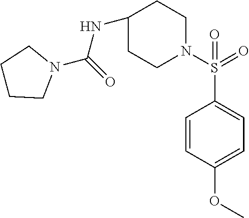 [SMILES: COc1ccc(S(=O)(=O)N2CCC(NC(=O)N3CCCC3)CC2)cc1] | 3.83 | 2.06 | >9.99 | >9.90 | — |
| PCM-0184891*^{/&} | 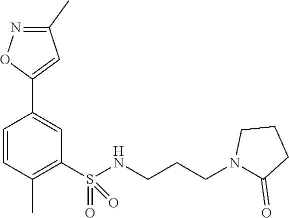 [SMILES: Cc1cc(-c2ccc(C)c(S(=O)(=O)NCCCN3CCCC3=O)c2)on1] | 2.37 | 3.26 | >9.99 | >9.90 | 218.6 |
| PCM-0184959*^{/&} | 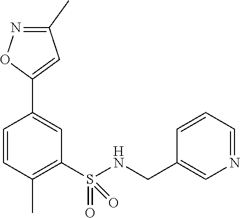 [SMILES: Cc1cc(-c2ccc(C)c(S(=O)(=O)NCc3cccnc3)c2)on1] | 2.16 | 2.72 | >9.99 | >9.90 | 249.4 |
| PCM-0185051*^{/&} | 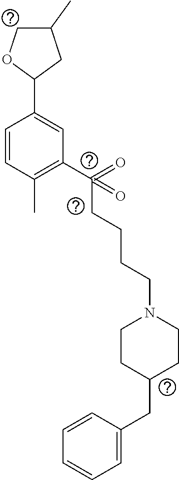 | 3.19 | 6.20 | >9.99 | >9.90 | — |
| PCM-0190999*^{/&} | 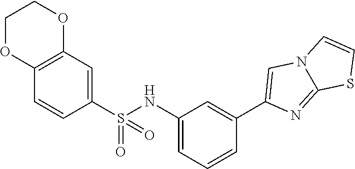 [SMILES: O=S(=O)(Nc1cccc(-c2cn3ccsc3n2)c1)c1ccc2c(c1)OCCO2] | 4.03 | 5.62 | <0.750 | <0.83 | — |
| PCM-0197024*^{/&} | 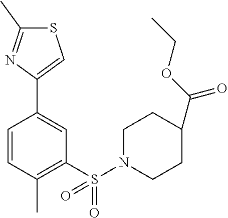 [SMILES: CCOC(=O)C1CCN(S(=O)(=O)c2cc(-c3csc(C)n3)ccc2C)CC1] | 3.20 | 4.16 | >9.99 | >9.90 | — |
| PCM-0200337*^{/&} | 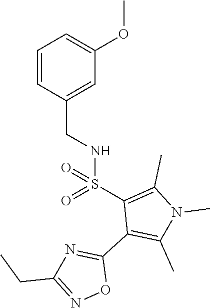 [SMILES: CCc1noc(-c2c(S(=O)(=O)NCc3cccc(OC)c3)c(C)n(C)c2C)n1] | 8.09 | >9.90 | >9.99 | >9.90 | — |
| PCM-0025239* | 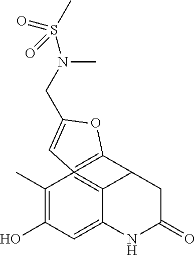 | — | 8.51 | — | 8.16 | — |
| PCM-0071569* | 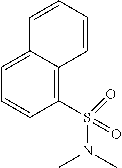 [SMILES: CN(C)S(=O)(=O)c1cccc2ccccc12] | — | >9.80 | — | >9.80 | — |
| PCM-0072788* | 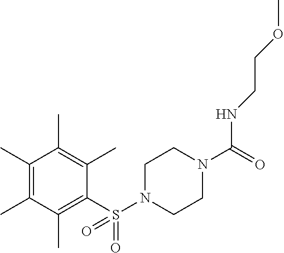 | — | >9.80 | — | >9.80 | — |
| PCM-0083647* | 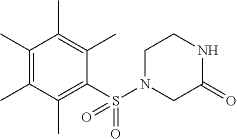 [SMILES: Cc1c(C)c(C)c(S(=O)(=O)N2CCNC(=O)C2)c(C)c1C] | — | 7.04 | — | >9.80 | — |
| PCM-0090313* | 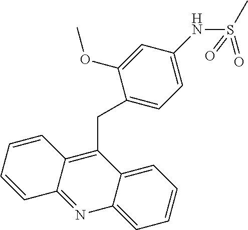 [SMILES: COc1cc(NS(C)(=O)=O)ccc1Cc1c2ccccc2nc2ccccc12] | — | >9.80 | — | >9.80 | — |
| PCM-0105406* | 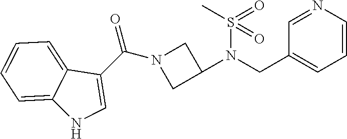 [SMILES: CS(=O)(=O)N(Cc1cccnc1)C1CN(C(=O)c2c[nH]c3ccccc23)C1] | 8.43 | >9.90 | >9.99 | >9.90 | — |
| PCM-0105813* | 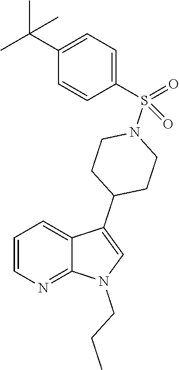 [SMILES: CCCn1cc(C2CCN(S(=O)(=O)c3ccc(C(C)(C)C)cc3)CC2)c2cccnc21] | >9.99 | >9.90 | >9.99 | >9.90 | — |
| PCM-0105851* | 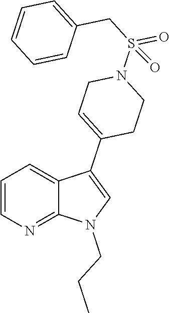 [SMILES: CCCn1cc(C2=CCN(S(=O)(=O)Cc3ccccc3)CC2)c2cccnc21] | 9.05 | >9.90 | >9.99 | >9.90 | — |
| PCM-0109036* | 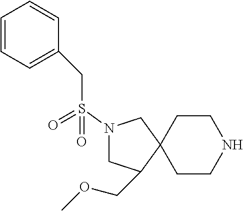 [SMILES: COCC1CN(S(=O)(=O)Cc2ccccc2)CC12CCNCC2] | >9.99 | >9.90 | >9.99 | >9.90 | — |
| PCM-0120253* | 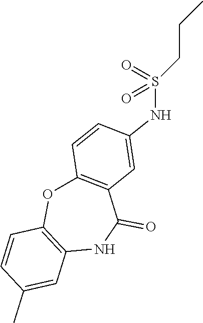 [SMILES: CCCS(=O)(=O)Nc1ccc2c(c1)C(=O)Nc1cc(C)ccc1O2] | 8.89 | >9.90 | >9.99 | 4.60 | — |
| PCM-0120563* | 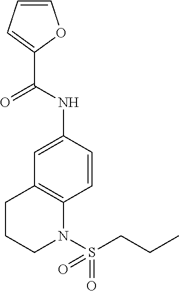 [SMILES: CCCS(=O)(=O)N1CCCc2cc(NC(=O)c3ccco3)ccc21] | >9.99 | >9.90 | >9.99 | >9.90 | — |
| PCM-0140113* | 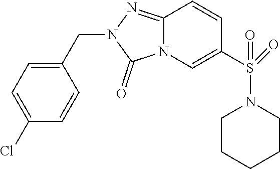 [SMILES: O=c1n(Cc2ccc(Cl)cc2)nc2ccc(S(=O)(=O)N3CCCCC3)cn12] | >9.99 | >9.90 | >9.99 | >9.90 | — |
| PCM-0172736* | 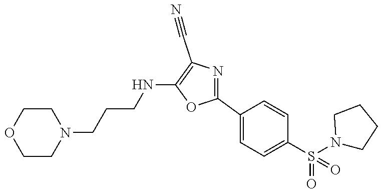 [SMILES: N#Cc1nc(-c2ccc(S(=O)(=O)N3CCCC3)cc2)oc1NCCCN1CCOCC1] | >9.99 | <0.83 | >9.99 | <0.83 | — |
| PCM-0172816* | 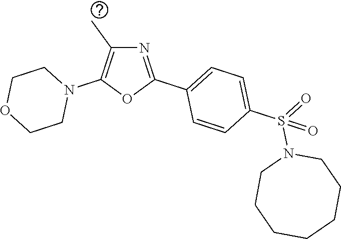 [SMILES: Cc1nc(-c2ccc(S(=O)(=O)N3CCCCCCC3)cc2)oc1N1CCOCC1] | >9.99 | <0.83 | >9.99 | <0.83 | — |
| PCM-0181527* | 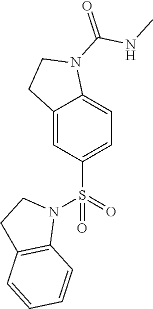 [SMILES: CNC(=O)N1CCc2cc(S(=O)(=O)N3CCc4ccccc43)ccc21] | >9.99 | >9.90 | >9.99 | >9.90 | — |
| PCM-0193675* | 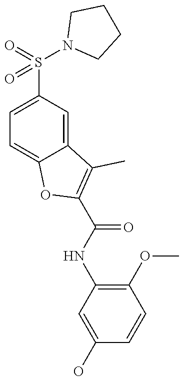 [SMILES: COc1ccc(O)cc1NC(=O)c1oc2ccc(S(=O)(=O)N3CCCC3)cc2c1C] | >9.99 | >9.90 | >9.99 | >9.90 | — |
| PCM-0195577* | 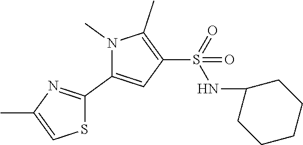 [SMILES: Cc1csc(-c2cc(S(=O)(=O)NC3CCCCC3)c(C)n2C)n1] | >9.99 | >9.90 | >9.99 | >9.90 | — |
| PCM-0206067* | 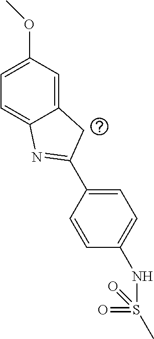 [SMILES: COc1ccc2c(c1)CC(c1ccc(NS(C)(=O)=O)cc1)=N2] | >9.99 | <0.83 | >9.99 | <0.83 | — |
| PCM-0207818* | 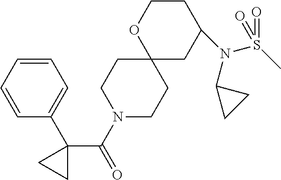 [SMILES: CS(=O)(=O)N(C1CC1)C1CCOC2(CCN(C(=O)C3(c4ccccc4)CC3)CC2)C1] | >9.99 | >9.90 | >9.99 | >9.90 | — |
| PCM-0124535*^{/#/&} | 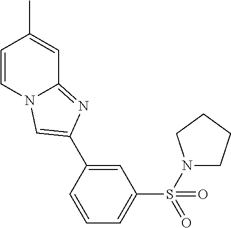 [SMILES: Cc1ccn2cc(-c3cccc(S(=O)(=O)N4CCCC4)c3)nc2c1] | 0.16 | 0.59 | >9.99 | >9.99 | 88.9 |
| PCM-0124812*^{/#/&} | 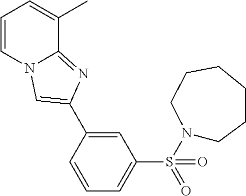 [SMILES: Cc1cccn2cc(-c3cccc(S(=O)(=O)N4CCCCCC4)c3)nc12] | 0.01 | 0.004 | 7.24 | 5.83 | 45.4 |
| PCM-0127506*^{/#/&} | 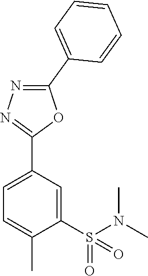 [SMILES: Cc1ccc(-c2nnc(-c3ccccc3)o2)cc1S(=O)(=O)N(C)C] | 1.620 | 2.202 | >9.99 | >9.99 | — |
| PCM-0127586**^{/#/&} | 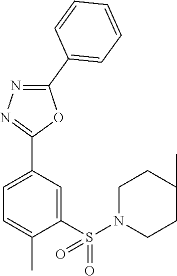 [SMILES: Cc1ccc(-c2nnc(-c3ccccc3)o2)cc1S(=O)(=O)N1CCC(C)CC1] | 1.20 | 5.97 | >9.99 | >9.99 | — |
| PCM-0186036*^{/#/&} | 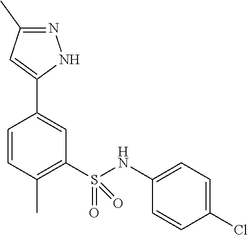 [SMILES: Cc1cc(-c2ccc(C)c(S(=O)(=O)Nc3ccc(Cl)cc3)c2)[nH]n1] | 1.16 | 2.36 | >9.99 | >9.99 | — |
| PCM-0197066*^{/#/&} | 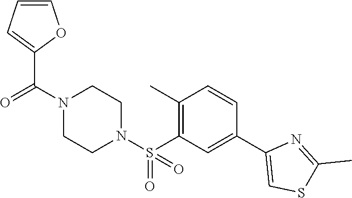 [SMILES: Cc1nc(-c2ccc(C)c(S(=O)(=O)N3CCN(C(=O)c4ccco4)CC3)c2)cs1] | 1.28 | 4.65 | >9.99 | >9.99 | — |
| PCM-0197104*^{/#/&} | 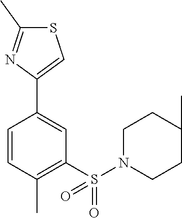 | 4.58 | 7.40 | >9.99 | >9.99 | — |
| PCM-0212385**^{/#/&} | 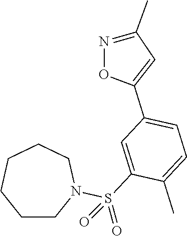 [SMILES: Cc1cc(-c2ccc(C)c(S(=O)(=O)N3CCCCCC3)c2)on1] | 1.24 | 1.92 | >9.99 | >9.99 | 23.8 |
| PCM-0212386**^{/#/&} | 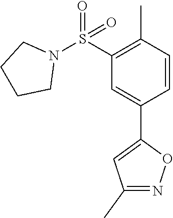 [SMILES: Cc1cc(-c2ccc(C)c(S(=O)(=O)N3CCCC3)c2)on1] | 0.75 | 0.42 | >9.99 | >9.99 | 65.1 |
| PCM-0212387**^{/#/&} | 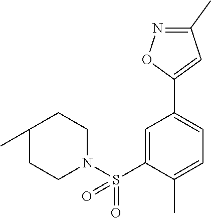 | 1.83 | 2.89 | >9.99 | >9.99 | 50.0 |
| PCM-0212388**^{/#/&} | 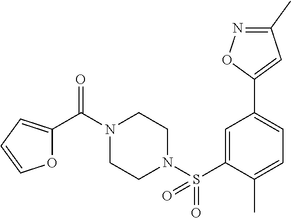 [SMILES: Cc1cc(-c2ccc(C)c(S(=O)(=O)N3CCN(C(=O)c4ccco4)CC3)c2)on1] | 1.25 | 1.19 | >9.99 | >9.99 | 123.5 |
| PCM-0212389**^{/#/&} | 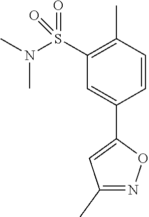 [SMILES: Cc1cc(-c2ccc(C)c(S(=O)(=O)N(C)C)c2)on1] | 1.71 | 2.31 | >9.99 | >9.99 | 215.1 |
| PCM-0213788**^{/#/&} | 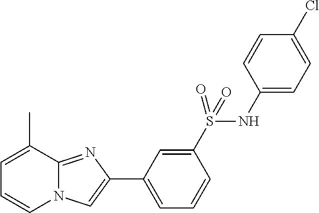 [SMILES: Cc1cccn2cc(-c3cccc(S(=O)(=O)Nc4ccc(Cl)cc4)c3)nc12] | 0.05 | 3.04 | >9.99 | >9.99 | — |
| PCM-0213790*^{/#/&} | 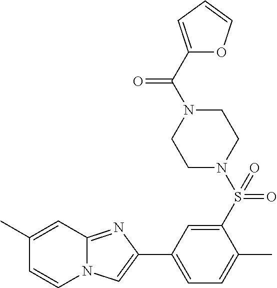 [SMILES: Cc1ccn2cc(-c3ccc(C)c(S(=O)(=O)N4CCN(C(=O)c5ccco5)CC4)c3)nc2c1] | 0.11 | 0.27 | 7.15 | >9.99 | — |
| PCM-0213791**^{/#/&} | 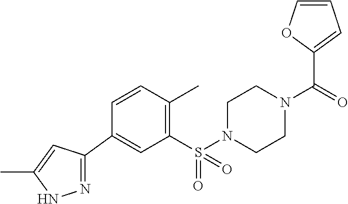 [SMILES: Cc1cc(-c2ccc(C)c(S(=O)(=O)N3CCN(C(=O)c4ccco4)CC3)c2)n[nH]1] | 0.08 | 0.26 | >9.99 | >9.99 | — |
| PCM-0213792**^{/#/&} | 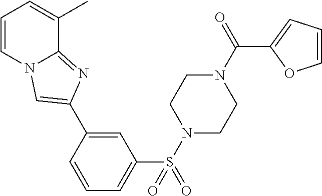 [SMILES: Cc1cccn2cc(-c3cccc(S(=O)(=O)N4CCN(C(=O)c5ccco5)CC4)c3)nc12] | 0.01 | 0.01 | 4.22 | 3.33 | 64.1 |
| PCM-0213793**^{/#/&} | 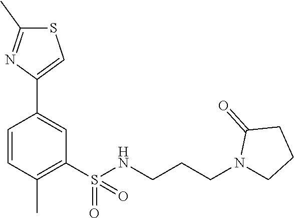 [SMILES: Cc1nc(-c2ccc(C)c(S(=O)(=O)NCCCN3CCCC3=O)c2)cs1] | 2.91 | 5.45 | >9.99 | >9.99 | — |
| PCM-0213794**^{/#/&} | 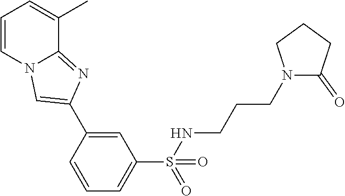 [SMILES: Cc1cccn2cc(-c3cccc(S(=O)(=O)NCCCN4CCCC4=O)c3)nc12] | 0.11 | 0.25 | >9.99 | >9.99 | — |
| PCM-0213795**^{/#/&} | 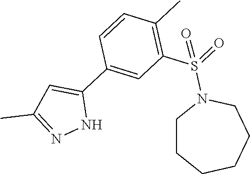 [SMILES: Cc1cc(-c2ccc(C)c(S(=O)(=O)N3CCCCCC3)c2)[nH]n1] | 0.28 | 0.64 | >9.99 | >9.99 | — |
| PCM-0213796**^{/#/&} | 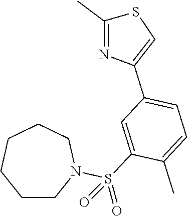 [SMILES: Cc1nc(-c2ccc(C)c(S(=O)(=O)N3CCCCCC3)c2)cs1] | 2.44 | >9.99 | >9.99 | >9.99 | — |
| PCM-0213797**^{/#/&} | 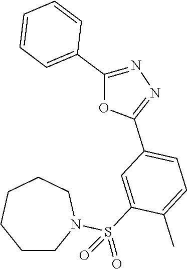 [SMILES: Cc1ccc(-c2nnc(-c3ccccc3)o2)cc1S(=O)(=O)N1CCCCCC1] | 1.06 | 3.38 | >9.99 | >9.99 | — |
| PCM-0213798**^{/#/&} | 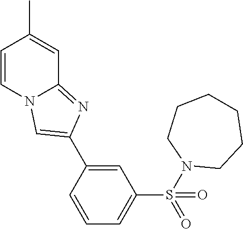 [SMILES: Cc1ccn2cc(-c3cccc(S(=O)(=O)N4CCCCCC4)c3)nc2c1] | 0.41 | 1.27 | >9.99 | >9.99 | — |
| PCM-0213799**^{/#/&} | 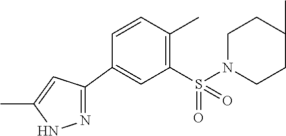 | 0.27 | 0.90 | >9.99 | >9.99 | — |
| PCM-0213800/ YB-800**^{/#/&/$} | 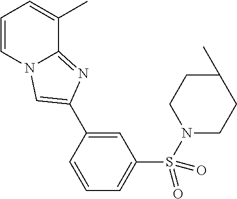 [SMILES: Cc1cccn2cc(-c3cccc(S(=O)(=O)N4CCC(C)CC4)c3)nc12] | 0.02 | 0.03 | >9.99 | >9.99 | 78.4 |
| PCM-0213801**^{/#/&} | 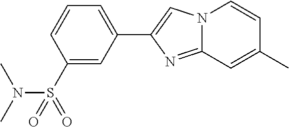 | 0.35 | 0.70 | >9.99 | >9.99 | — |
| PCM-0213802**^{/#/&} | 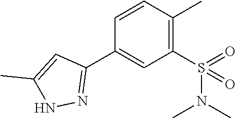 [SMILES: Cc1cc(-c2ccc(C)c(S(=O)(=O)N(C)C)c2)n[nH]1] | 0.16 | 0.39 | >9.99 | >9.99 | — |
| PCM-0213803**^{/#/&} | 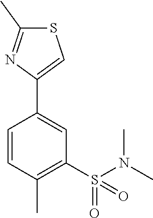 | 0.10 | 2.80 | >9.99 | >9.99 | — |
| PCM-0213804**^{/#/&/$} | 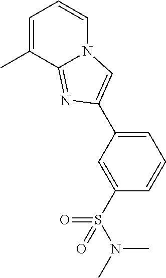 [SMILES: Cc1cccn2cc(-c3cccc(S(=O)(=O)N(C)C)c3)nc12] | 0.02 | 0.19 | >9.99 | >9.99 | >100.0 |
| PCM-0213805*^{/#/&} | 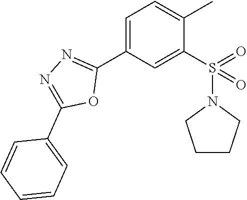 [SMILES: Cc1ccc(-c2nnc(-c3ccccc3)o2)cc1S(=O)(=O)N1CCCC1] | 0.25 | 1.48 | >9.99 | >9.99 | — |
| PCM-0213806**^{/#/&/$} | 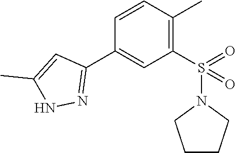 [SMILES: Cc1cc(-c2ccc(C)c(S(=O)(=O)N3CCCC3)c2)n[nH]1] | 0.05 | 0.17 | >9.99 | >9.99 | — |
| PCM-0213807**^{/#/&} | 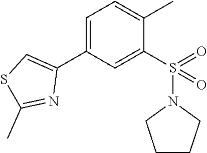 [SMILES: Cc1nc(-c2ccc(C)c(S(=O)(=O)N3CCCC3)c2)cs1] | 0.37 | 1.55 | >9.99 | >9.99 | — |
| PCM-0213808/ YB-808**^{/#/&/$} | 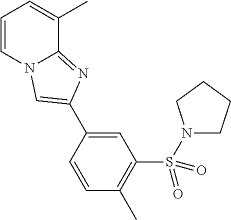 [SMILES: Cc1ccc(-c2cn3cccc(C)c3n2)cc1S(=O)(=O)N1CCCC1] | 0.01 | 0.01 | >9.99 | >9.99 | 59.7 |
| PCM-0213809**^{/#/&} | 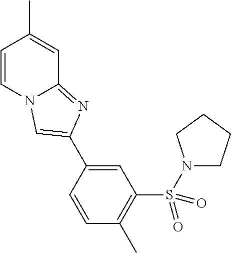 [SMILES: Cc1ccn2cc(-c3ccc(C)c(S(=O)(=O)N4CCCC4)c3)nc2c1] | 0.18 | 0.51 | >9.99 | >9.99 | — |
| PCM-0213811**^{/#/&} | 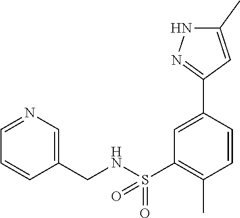 [SMILES: Cc1cc(-c2ccc(C)c(S(=O)(=O)NCc3cccnc3)c2)n[nH]1] | 0.98 | 3.16 | >9.99 | >9.99 | — |
| PCM-0213812**^{/#/&} | 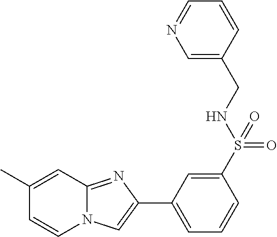 [SMILES: Cc1ccn2cc(-c3cccc(S(=O)(=O)NCc4cccnc4)c3)nc2c1] | 0.75 | 9.62 | 8.60 | >9.99 | — |
| PCM-0213813**^{/#/&} | 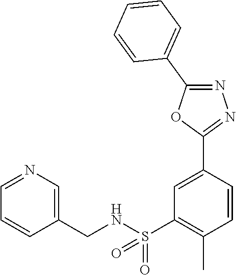 [SMILES: Cc1ccc(-c2nnc(-c3ccccc3)o2)cc1S(=O)(=O)NCc1cccnc1] | 1.17 | 6.49 | >9.99 | >9.99 | — |
| PCM-0213814**^{/#/&/$} | 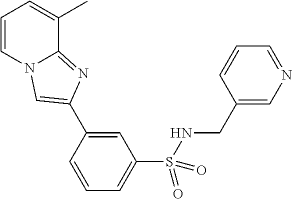 [SMILES: Cc1cccn2cc(-c3cccc(S(=O)(=O)NCc4cccnc4)c3)nc12] | 0.02 | 0.02 | 7.18 | >9.99 | >99.0 |
| PCM-0213815**^{/#/&} | 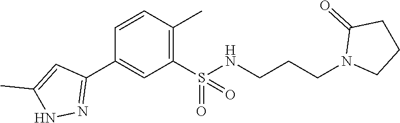 [SMILES: Cc1cc(-c2ccc(C)c(S(=O)(=O)NCCCN3CCCC3=O)c2)n[nH]1] | 0.28 | 0.93 | >9.99 | >9.99 | — |
| PCM-0213817**^{/#/&} | 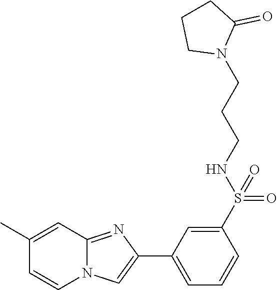 [SMILES: Cc1ccn2cc(-c3cccc(S(=O)(=O)NCCCN4CCCC4=O)c3)nc2c1] | 0.32 | 0.70 | 3.44 | 5.28 | — |
| PCM-0214338**^{/#/&/$} | 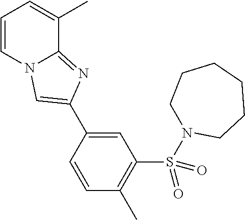 [SMILES: Cc1ccc(-c2cn3cccc(C)c3n2)cc1S(=O)(=O)N1CCCCCC1] | 0.03 | 0.02 | >9.99 | >9.99 | >99.0 |
| PCM-0214540/ YB-540**^{/#/&/$$} | 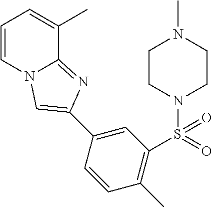 [SMILES: Cc1ccc(-c2cn3cccc(C)c3n2)cc1S(=O)(=O)N1CCN(C)CC1] | — | 0.425 | — | >9.99 | — |
| PCM-0220537/ YB-537**^{/#/*/$$} | 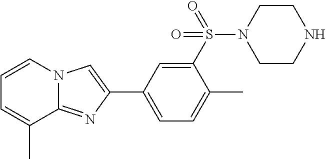 [SMILES: Cc1ccc(-c2cn3cccc(C)c3n2)cc1S(=O)(=O)N1CCNCC1] | — | 0.03 | — | 18.4 | >99.0 |
| PCM-0214339**^{/#/$} | 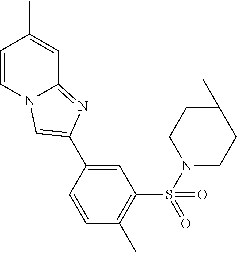 [SMILES: Cc1ccn2cc(-c3ccc(C)c(S(=O)(=O)N4CCC(C)CC4)c3)nc2c1] | 4.49 | 3.96 | — | | 59.4 |

### Example 1

### UTS-Identified Sulfonamide Compounds as Novel, Highly Potent, Selective and Soluble QR2 Inhibitors

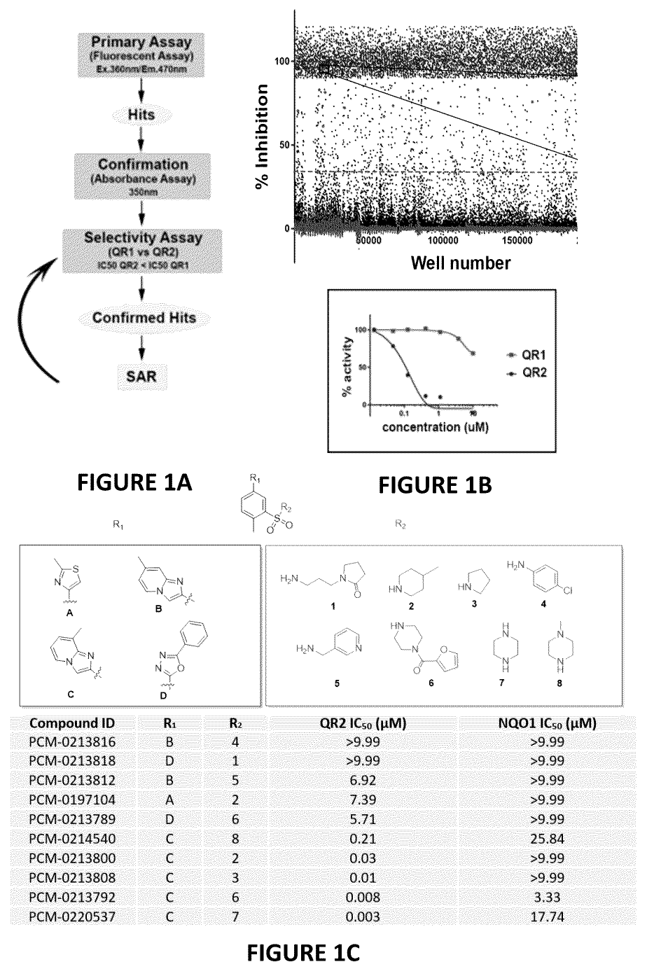

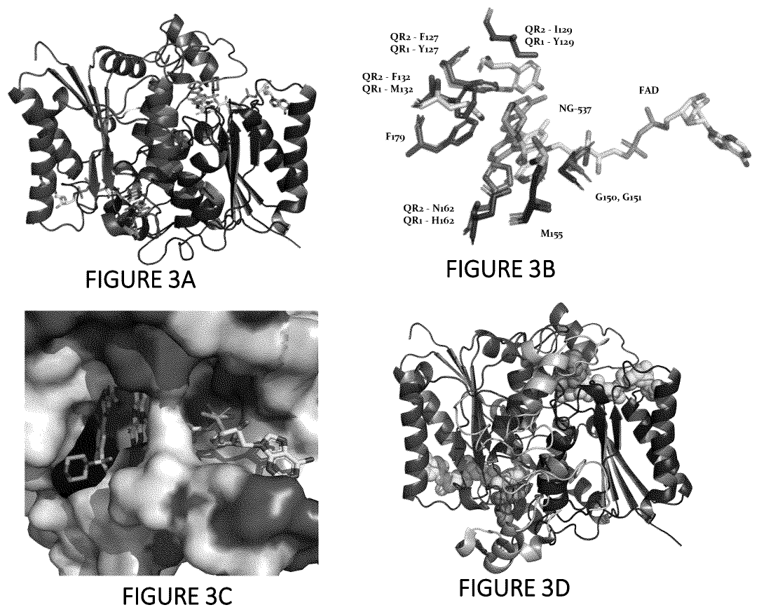

A drug discovery campaign strategy was selected, in which initially screened chemicals would be evaluated for specific cell free QR2 inhibition and in vitro activity, selected for SAR development and then cyclically evaluated (FIG. 1A) to achieve highly selective and potent QR2 inhibitor synthesis. In order to establish a varied, flexible and chemically amenable starting point from which to develop such inhibitors, the inventors ran a standardized QR2 assay (see methods) against ˜200,000 compounds, from a varied assortment of chemical libraries. Compounds that induced >30% inhibition were then selected for validation, using a dose response in both the standard assay using BNAH co factor fluorescence decay as a readout, as well as an orthogonal assay, in which BNAH absorption was measured instead (see methods). Compounds that replicated the initial hit result were then assayed similarly, against NQO1, to evaluate compound specificity (FIG. 1B). From the HTS, a series of compounds containing a sulfonamide central linker, with an amine on one side and a heterocycle on the other, were identified and selected for SAR development (FIG. 1C). Analysis of the SAR data revealed that the imidazopyridine heterocycle was essential for activity of the sulfonamide series and proved to be superior to other heterocycles that were evaluated (FIG. 1C), forming crucial Pi-Pi interaction with the protein, attributing to its potency, as was later found (FIG. 3). Variation of the amine in the sulfonamide series was much more tolerable and aliphatic primary and secondary amines gave good activity and selectivity, noting that anilines proved to be less active. Thus, a number of highly selective and potent QR2 inhibitors were made, allowing breadth and scope for future modifications and further improvement, based on the structures identified and developed.

### Example 2

### QR2 Inhibitors Bind Target In Vitro and Reproduce QR2 KO Results in Isogenic Controls

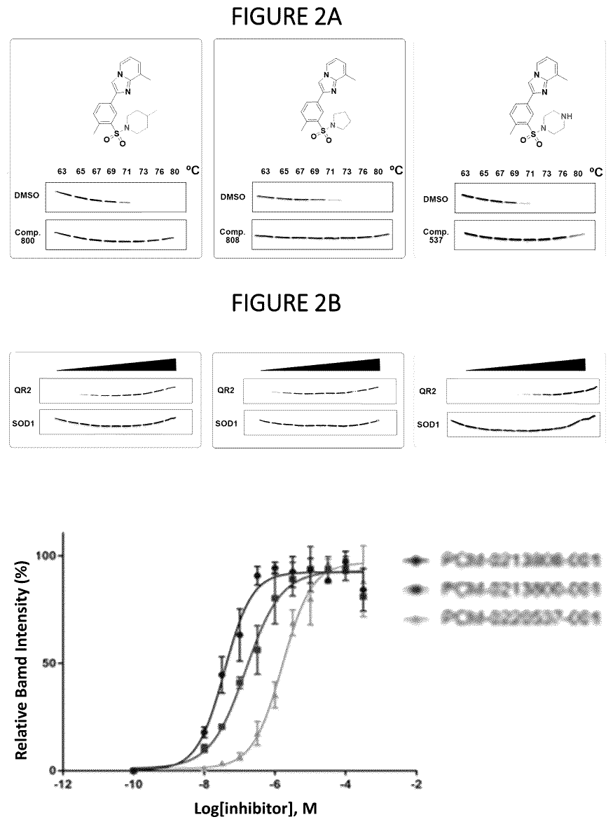

To establish direct target engagement, as well as cell membrane permeability, a cellular thermal shift assay (CETSA) was carried out (see methods), using the leading compounds YB-537, YB-800, and YB-808. Following 1 h preincubation with 5 μM of each of the compounds, or vehicle, thermal aggregation curves (Tagg) of QR2 expressed in HEK293T were measured in increasing temperatures. All compounds showed shifts of thermal stability of QR2 when compared to the vehicle (FIG. 2A). Based on the Tagg curves, the inventors selected 73° C. for isothermal dose-response fingerprint (IDTRF) experiments (FIG. 2B). The ITDRF allowed the relative quantification of the binding of each of the compounds to QR2, measured as the half-maximal effective concentration (EC50), to be 129, 34 and 13 nM for YB-537, 800 and 808, respectively. This shows that the novel inhibitors are both able to penetrate the cell membrane and directly bind the target QR2 protein within.

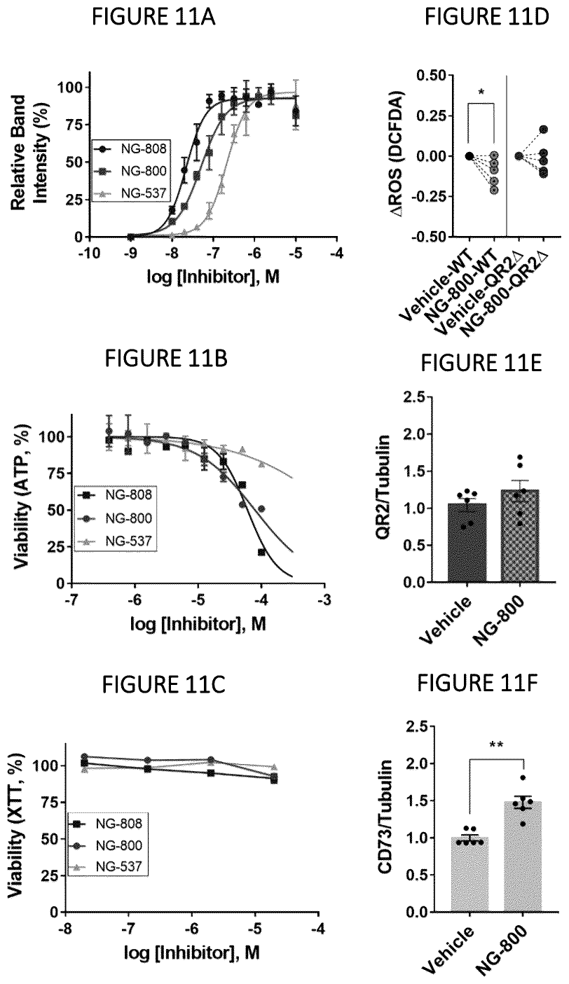

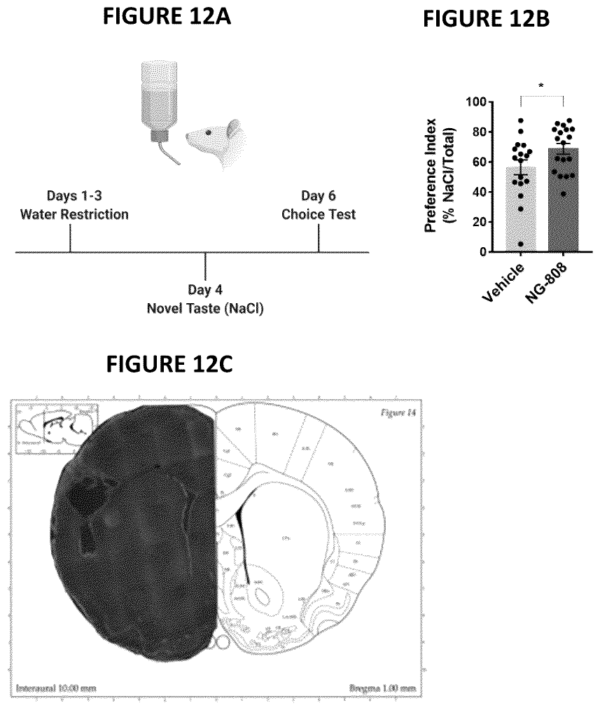

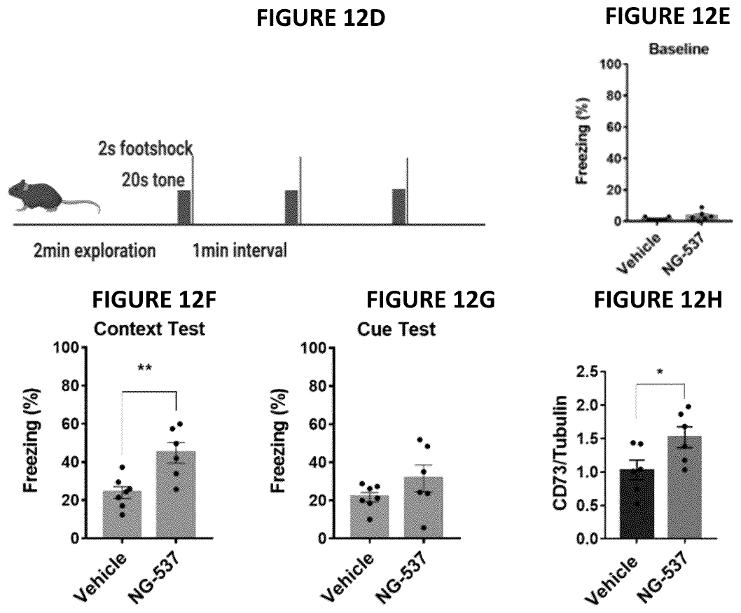

The inventors then aimed to evaluate the safety of the inhibitors with the use of cell toxicity and viability assays. First, toxicity was assessed in an ATP depletion assay, using THLE-2 cells following a 72 h exposure to a dose response of each of the tested compounds. Only one of the tested compounds, exhibited an IC50<10 μM (PCM-0212354; Table 3), while the leading inhibitors displayed much higher values (FIG. 11B), exemplified by YB-537. Additionally, cell viability was assessed in HEK239T cells, using the XTT assay, with increasing doses of the leading inhibitors showing no sign of toxicity following 3 h of incubation, using relevant doses (FIG. 11C). Together, these results demonstrate that the newly synthesized QR2 inhibitors possess extremely low toxicity and are able to effectively engage the target in vitro. Next, the inventors wanted to assess the ability of the inhibitors to replicate the effect of QR2 KO in the WT isogenic control HCT116 cells, and to see whether any observable effect is occluded in the QR2Δ HCT116 cells. To do so, the inventors used YB-800, which showed an EC50 intermediate to YB-808 and YB-537 (FIG. 11A). The inventors found that ROS levels were significantly reduced 3 h following acute treatment with 20 μM YB-800 in the WT but not in the QR2Δ HCT116 cells (FIG. 3d), indicating a QR2 inhibition specific effect of the inhibitor, which recaptured the ROS phenotype found in the QR2Δ cells. The inventors then assessed WT HCT116 cells by immunoblot, following a 4 day, daily treatment with YB-800 (2 μM). QR2 expression (antibody: Santa Cruz, sc-271665) was unaltered (FIG. 11E), however CD73 (antibody: Cell-Signaling, D7F9A) showed a significant increase (FIG. 11F), similarly to QR2Δ (Supplementary FIG. 3), in which CD73 was one of the most strongly up-regulated proteins. Thus, the QR2 inhibitors are able to bind native QR2 in human cells and replicate the effects seen using QR2 genetic interference.

### Example 3

### Crystal Structure of YB-537 Bound to QR2

To determine ligand-target interactions, purified human QR2 was co-crystalized in the presence of YB-537 (FIG. 3A). The resolved structure shows QR2 is a physiological homodimer composed of 231 amino acids per monomer, and has a/P folds with flavodoxin topology, with two FAD molecules situated at the two extremes of the dimer interface, as previously described. YB-537 is bound to each of the monomers, and interacts with amino acids from both monomers, as well as with FAD. The plane of YB-537 stacks up parallel to the isoalloxazine ring of the FAD and the average distance between the planes of the two rings is 3.5 Å. Each FAD moiety forms 17 contacts within 3.5 Å to atoms from YB-537 and 48 contacts to 17 amino acids from one monomer H12, S17, F18, N19, S21, P103, L104, Y105, W106, F107, T148, T149, G150, G151, Y156, E194, and R201. YB-537 binds to the catalytic site through a series of hydrophobic and hydrogen bonds with both FAD and amino acids from both QR2 monomers, in a manner unique to it (FIG. 3B). Specifically, YB-537 forms 6 contacts to G149, G150, M154 and a hydrogen bond with N161 from one monomer A and 8 contacts to F126, I128, F131, and F178 from monomer B.

To estimate the evolutionary conservation of QR2 amino acids based on the phylogenetic relation between homologous sequences, the inventors applied the ConSurf server (Ashkenazy et al., 2016). The server produced multiple sequence alignment of 150 human QR2 related proteins, clearly detecting the high conservation among the amino acids interacting with FAD (represented in maroon in FIG. 3C), while showing far less conservation with those interacting with YB-537 (represented in turquoise in FIG. 3C). Superposition of the catalytic site of QR2, in complex with YB-537, with NQO1 (PDB-ID code 2F10; FIG. 3B) reveal that while some catalytic site amino acids are strictly conserved, 3 amino acids are conserved among the QR2 family members but not in NQO1 (FIG. 3B). Specifically, I128, F131 and N161 in QR2 are Tyr, Met and His respectively in NQO1. This may confer selectivity to QR2, as the Y128 and H161 amino acids in NQO1 are in close proximity to YB-537, and therefore, present a physical obstacle for inhibitor binding. Additionally, the F131 ring in QR2 is parallel to the 6-member ring (6 membered ring with 2 nitrogen atoms) of YB-537, while M131 in NQO1 lacks these interactions. This, as well as the presence of a flexible 43-residue C-terminal tail in NQO1 which might obstruct access to the catalytic site (FIG. 3D), may explain the observed many fold higher binding affinity of YB-537 to QR2 compared to that of NQO1.

### Example 4

### QR2 Inhibitors are Non-Toxic

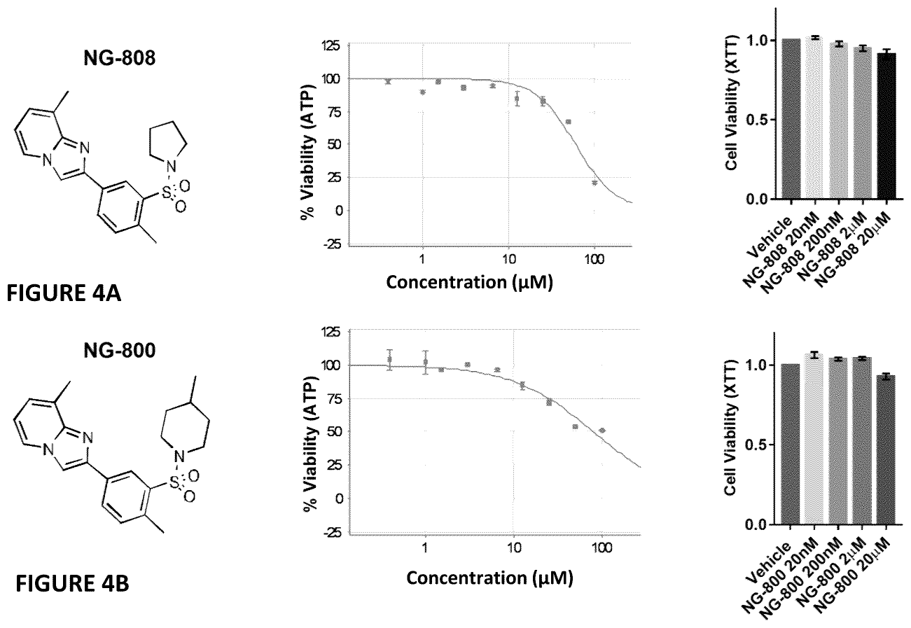

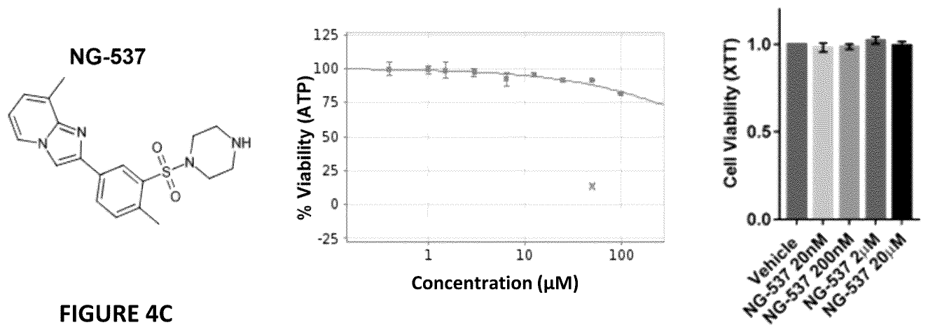

Next, the inventors examined the safety of the inhibitors with the use of cell toxicity and viability assays. First, toxicity was assessed in an ATP depletion assay, using THLE2 following exposure to a dose response of each of the tested compounds following a 72-hour period. Only one of the tested compounds, exhibited an IC50<10 μM (PCM-0212354). Additionally, cell viability was assessed in HEK239T cells, using the XTT assay, with increasing doses of the inhibitors showing no sign of toxicity following 3 h of incubation. Together, these results demonstrated that the newly synthesized QR2 inhibitors possess extremely low toxicity, and are therefore, safe to be used in preclinical trials (FIG. 4).

### Example 5

### Novel QR2 Inhibitors Enhance Cortical and Hippocampal Learning In Vivo

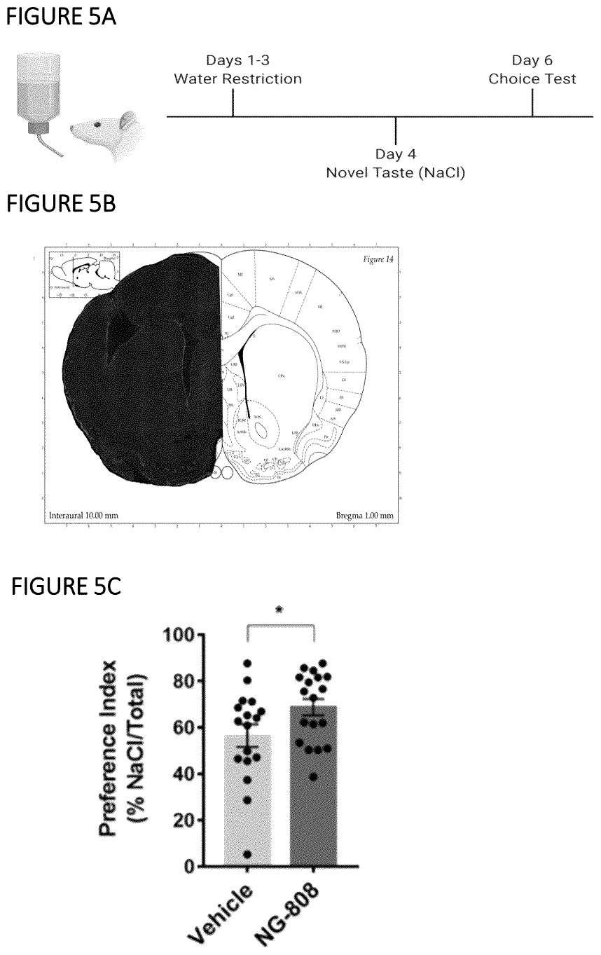

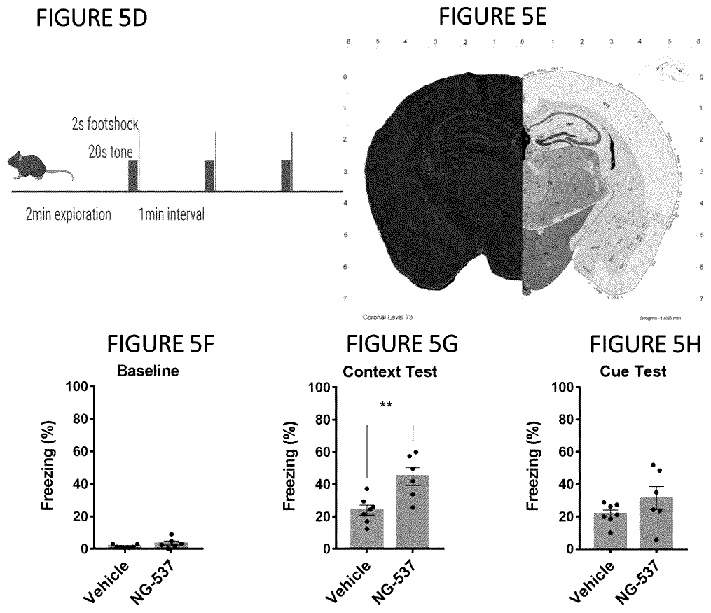

To evaluate the efficacy of the leading inhibitors in vivo, separate double blind experiments were carried out, in two different modalities, involving different brain areas. First, cortical memory was tested using incidental novel taste learning (Yiannakas and Rosenblum, 2017). In this paradigm, the innate neophobic response exhibited toward a new, unfamiliar taste is utilized. Taste neophobia is highly conserved across species, as it is the first line of defense against potentially poisonous, newly discovered foodstuffs. Hence, when an animal first encounters a new taste, it consumes very little. However, once a memory for the taste is formed and no associated ill effect is learned, the taste becomes familiar, and the animal consumes it more freely. Thus, the stronger the memory for the safe taste, the more likely the animal will consume it. Therefore, in order to test whether the novel inhibitors could help rats remember a novel taste and enhance their memory for it, rats were cannulated to the anterior insular cortex (aIC; FIG. 5B) and allowed 7 days to recover. Then, the animals were taught to drink from pipettes, during a restricted period of 20 min, and were then given a novel taste (0.3% NaCl). Two days later, the memory for the incidentally learned novel taste was evaluated by measuring the preference index of the taste (see methods; FIG. 5A). Animals that received YB-808 (20 μM) drank significantly more NaCl than the control group, therefore displaying a stronger memory of the safe taste (FIG. 5C). Next, hippocampal dependent learning was evaluated in response to infusion of the novel inhibitors. First, mice were cannulated to CA1 (FIG. 5E) and were given 7 days to recover, prior to undergoing delay fear conditioning (DFC) 20 min following infusion of YB-537 (5 μM, FIG. 5D). This conditioning paradigm involves hippocampal CA1 dependent learning of the context, and amygdala dependent learning of the cue (Phillips and LeDoux, 1992). Namely, animals are allowed to explore the conditioning chamber for 2 min, during which time baseline freezing is measured (FIG. 5F). Then, a tone (conditioned stimulus, CS) is played for 20 s and ends as a foot shock (unconditioned stimulus, US) is given. A 1 min interval separates this CS-US pairing, as it occurs for a total of three times. Mice, which have now associated the foot shock with the tone and the context can be tested for both hippocampal and amygdala dependent memory, the latter of which provides an internal control, as mice only received YB-537 to the hippocampus, and not the amygdala. The day after conditioning, mice were returned to the chamber and freezing in response to the context was measured, providing a proxy for the strength of the memory formed. Animals that received YB-537 to CA1 froze significantly more in response to the conditioned context, displaying stronger recollection and hippocampal dependent memory (FIG. 5G). The following day, the animals were tested for memory of the cue, in a different context. No significant difference in the level of increased freezing was seen between the groups (FIG. 5H), indicating no change to amygdala dependent memory. Together, these double blind behavioral experiments in rats and mice demonstrated that the novel QR2 inhibitors are able to enhance long-term memory across brain regions, in different learning modalities.

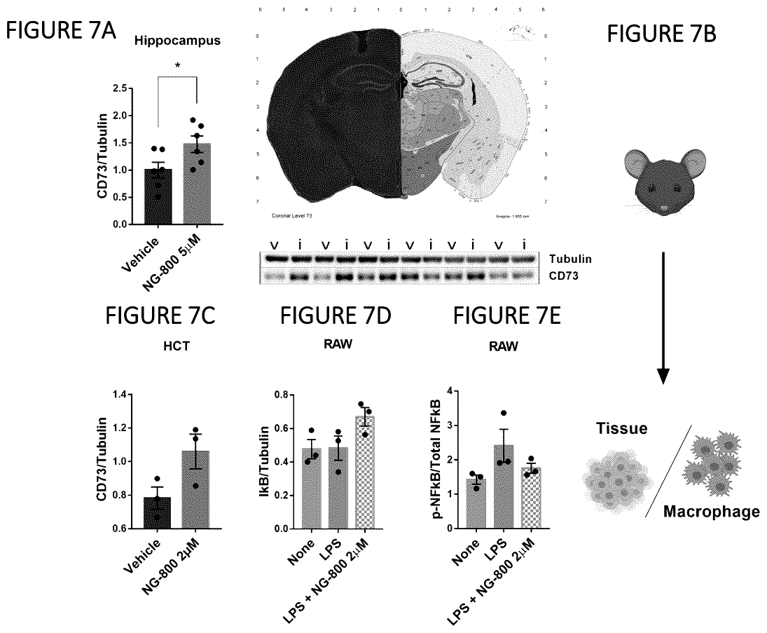

The mice were then given a week to rest hand were then microinjected with 1 μL of 5 μM YB-537 to CA1 for a further 5 days, once a day at the same time. At the end of this period, the mice were sacrificed and the brains were flash frozen in liquid nitrogen. CA1 samples were dissected and immunoblots for CD73 were done in order to compare with the results obtained in human cells, except for one brain that was used to validate cannula placement (FIG. 7A). Similarly to previous experiments, QR2 inhibition in the mouse CA1 also significantly increased CD73 levels (FIGS. 7A, and 7C, antibody: Cell-Signaling, D7F9A), corroborating the proteomics result and in vitro validation in human cells. These experiments demonstrate the ability of the novel QR2 inhibitors to replicate a result previously obtained in vitro in the brain, whilst improving learning and memory tasks similarly to genetically induced QR2 elimination, across species.

### Example 6

QR2 KO in a Human Cell Line Induces Functional Proteomic Changes Antagonistic to that of Alzheimer's Disease Patients Cortex

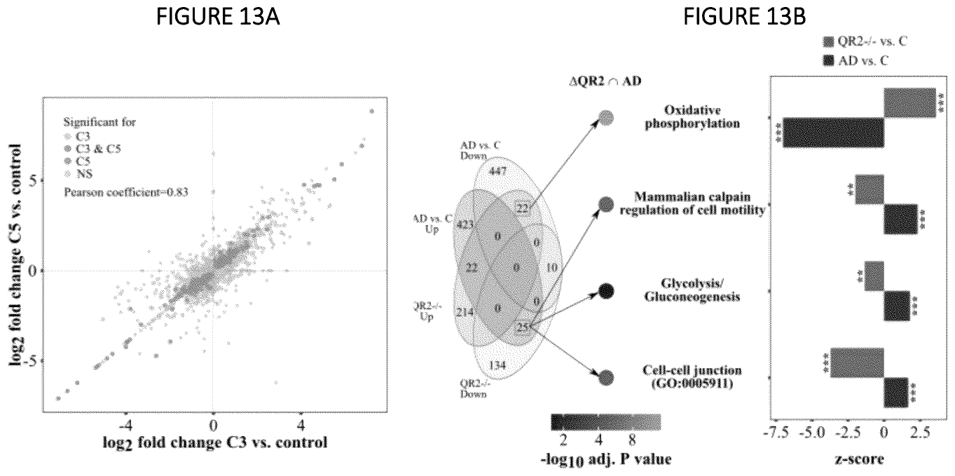

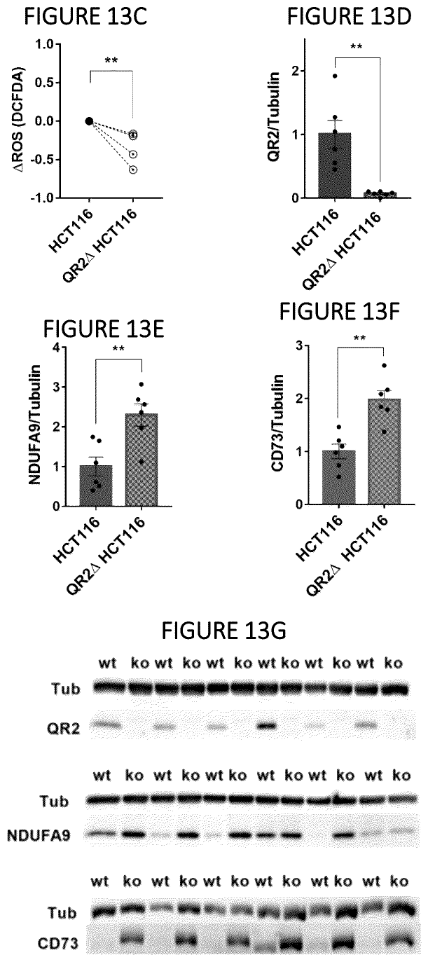

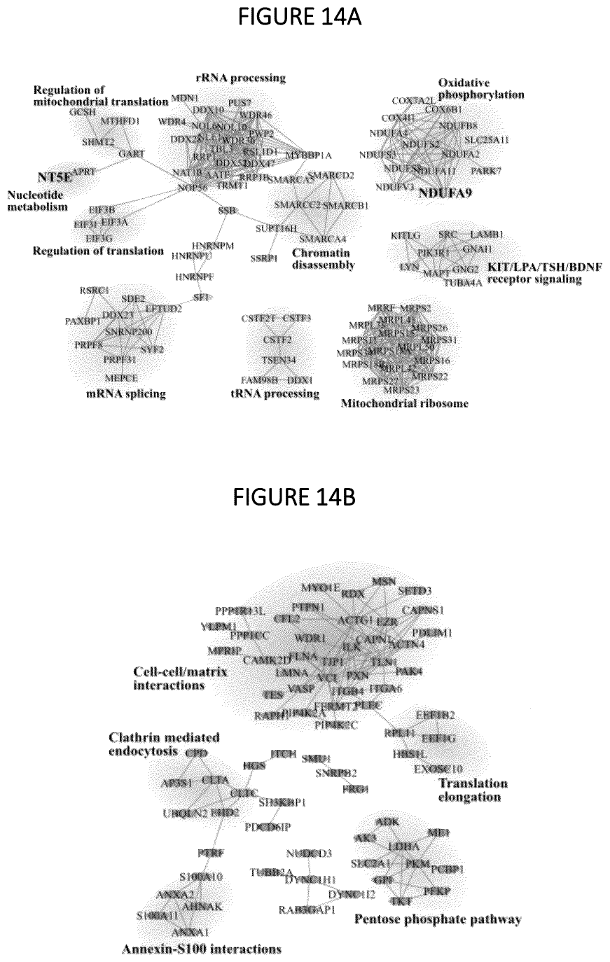

There is evidence of QR2 mediated metabolic stress in human cells, however it is not yet known how QR2 activity may affect the proteome in response to the mild chronic stress it generates. The inventors therefore used CRISPR mediated QR2 knockout (KO) in HCT116 cells (QR2Δ cells). Briefly, exon 4 of QR2 was disrupted using a dual CRISPR-Cas9 nickase system to reduce off-target cleavage. After treatment with the CRISPR-Cas9 nickase system, individual cells were amplified to yield 5 cell lines. One of these, Clone 1 (C1) had an intact QR2 exon 4 and expressed QR2 protein; C1 was retained as an isogenic “parental” QR2-expressing control. The other 4 cell lines, C2 through C5, lacked QR2 by western blot (WB); of these, C3 and C5 had a clearly defined deletion in QR2 exon 4, but in C2 and C4 exon 4 appeared completely absent, possibly due to a chromosomal translocation. Details of the knockout procedure and corresponding figures have been previously published (reference). Proteomic analysis (see methods) showed that both C3 and C5 produced highly similar results when compared to the isogenic control, C1 (FIG. 13A). The comparison provided a number of functional groups of proteins that significantly differed following QR2 KO. QR2 KO affected expression of mitochondrial proteins (FIG. 14) including increased expression of mitochondrial mRNA translation (i.e., mitochondrial ribosome proteins, regulation of mitochondrial mRNA translation) and oxidative phosphorylation gene products. The majority of the latter are NADH dehydrogenase proteins. Other groups of proteins with increased expression in QR2Δ were related to mRNA transcription and translation. In parallel to increased oxidative phosphorylation protein expression, proteins involved in glycolysis and pentose phosphate pathway had decreased expression in QR2Δ (Supplementary FIG. 1). Many other QR2Δ downregulated proteins were involved in cell-cell junctions and cell-matrix interactions. Interestingly, while mRNA translation initiation regulators were upregulated in QR2A, mRNA translation elongation factors were downregulated. Since impairment of oxidative phosphorylation as well as cell-cell interactions are among the leading processes involved in AD, the inventors sought to compare the QR2Δ proteome to proteomes of human brain tissue that test AD vs. control. A distinct antagonistic profile is seen between QR2Δ and AD (FIG. 13B), with oxidative phosphorylation-related proteins being the dominant group of proteins with contrasting effects between QR2Δ and AD. Importantly, among overlapping cell-cell junction proteins were the two Calpain 1 subunits (CAPNS1 and CAPN1). This protease is of critical importance for synaptic plasticity and is suggested to be involved in Alzheimer's disease pathogenesis (e.g., functioning as a tau protease). The gene-set enrichment information found here therefore points to a possible contribution of QR2 to the AD phenotype. In agreement with the CRISPRi manipulation, QR2 (antibody: Santa Cruz, sc-271665) was not detected by immunoblot in the QR2Δ cells (FIG. 13D), and in agreement with previous QR2 interference studies, a significant reduction in cellular ROS levels is seen in the QR2Δ cells compared to the isogenic controls (FIG. 13C). In order to further validate the results found in the proteomic analysis, two targets corresponding to some of the functional groups identified were chosen, based on availability of reliable antibodies, including NDUFA9 (mitochondrial respiration, complex I subunit, antibody: AbCam, ab14713) and CD73 (nucleotide metabolism and inflammation, antibody: Cell-Signaling, D7F9A). Using WB and direct measurement, the inventors found that, in agreement with the LC-MS results obtained, both NDUFA9 (FIG. 13E) and CD73 (FIG. 13F) were significantly increased in QR2Δ cells compared to their isogenic controls. Since genetic interference of QR2 provides a proteomic profile antagonistic to AD, causes a reduction in metabolic stress and QR2 has been previously linked with AD and cognitive function, it follows that QR2 inhibition is an attractive and novel candidate for AD drug development.

### Example 7

YB-537 Bound to Human QR2 Shows Conserved Ligand-Target Interactions that are Absent in the Closely Related QR1

In order to enable water-solubility so that an inhibitor could be easily applied to crystalized QR2, and to enable oral administration of the inhibitor and eliminate undesirable formulations, HCl was conjugated to YB-537 (see step 6 in methods section ‘Synthesis of Novel QR2 Inhibitors’ and Table 3). This resulted in complete solution of YB-537 in water. Using the water-soluble YB-537 to determine ligand-target interactions, purified human QR2 was co-crystalized in the presence of the inhibitor (FIG. 3A). The resolved structure shows QR2 is a physiological homodimer composed of 231 amino acids per monomer, and has a/P folds with flavodoxin topology, with two FAD molecules situated at the two extremes of the dimer interface, as previously described. YB-537 is bound to each of the monomers, and interacts with amino acids from both monomers, as well as with FAD. The plane of YB-537 stacks up parallel to the isoalloxazine ring of the FAD and the average distance between the planes of the two rings is 3.5 Å. Each FAD moiety forms 17 contacts within 3.5 Å to atoms from YB-537 and 48 contacts to 17 amino acids from one monomer, namely—H12, S17, F18, N19, S21, P103, L104, Y105, W106, F107, T148, T149, G150, G151, Y156, E194, and R201. YB-537 binds to the catalytic site through a series of hydrophobic and hydrogen bonds with both FAD and amino acids from both QR2 monomers, in a manner unique to it (FIG. 3B). Specifically, YB-537 forms 6 contacts to G150, G151, M155 and a hydrogen bond with N162 from one monomer (A) and 8 contacts to F127, 1129, F132, and F179 from the other monomer (B).

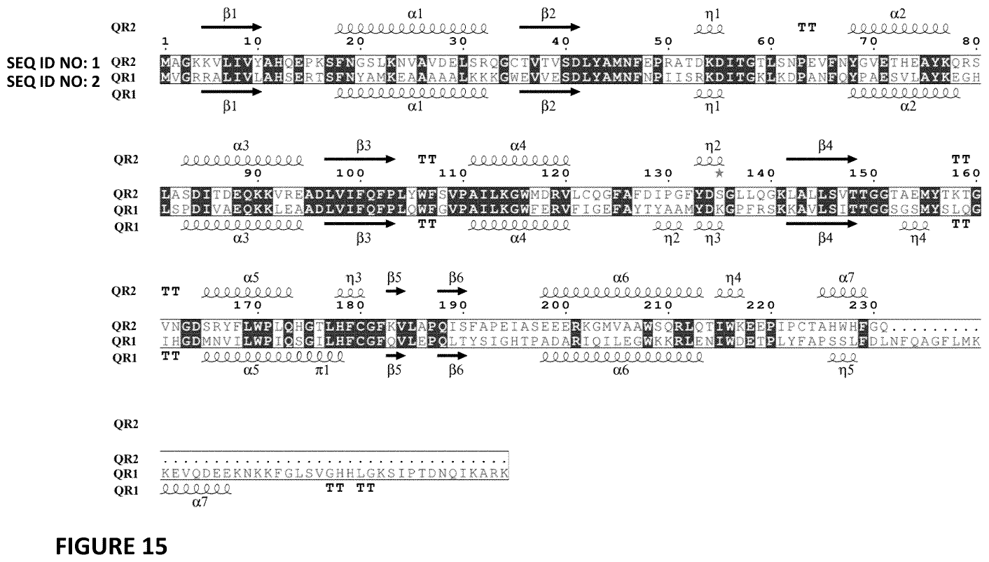

To estimate the evolutionary conservation of QR2 amino acids based on the phylogenetic relation between homologous sequences the inventors applied the ConSurf server. The server produced multiple sequence alignment of 150 human QR2 related proteins, clearly detecting the high conservation among the amino acids interacting with FAD (represented in maroon in FIG. 3C), while showing far less conservation with those interacting with YB-537 (represented in turquoise in FIG. 3C). Superposition of the catalytic site of QR2, in complex with YB-537, with QR1 (PDB-ID code 2F10; FIG. 3B) reveal that while some catalytic site amino acids are strictly conserved, three amino acids are conserved among the QR2 family members but not in QR1 (FIG. 3B). Specifically, 1129, F132 and N162 in QR2 are Tyr, Met and His respectively in QR1. This may confer selectivity to QR2, as the Y129 and H162 amino acids in QR1 are in close proximity to YB-537 and therefore present a physical obstacle for inhibitor binding. Additionally, the F132 ring in QR2 is parallel to the 6-member ring (which includes 2 nitrogen atoms) of YB-537, while M132 in QR1 lacks these interactions. This, as well as the presence of a flexible 43-residue C-terminal tail in QR1 (FIG. 15) which might obstruct access to the catalytic site (FIG. 5d), may explain the observed >6,000-fold higher binding affinity of YB-537 to QR2 compared to QR1.

### Example 8

### Ingestion of YB-537 in Drinking Water Significantly Improves Cognitive Function in 8-9 Months Old 5×FAD Mice

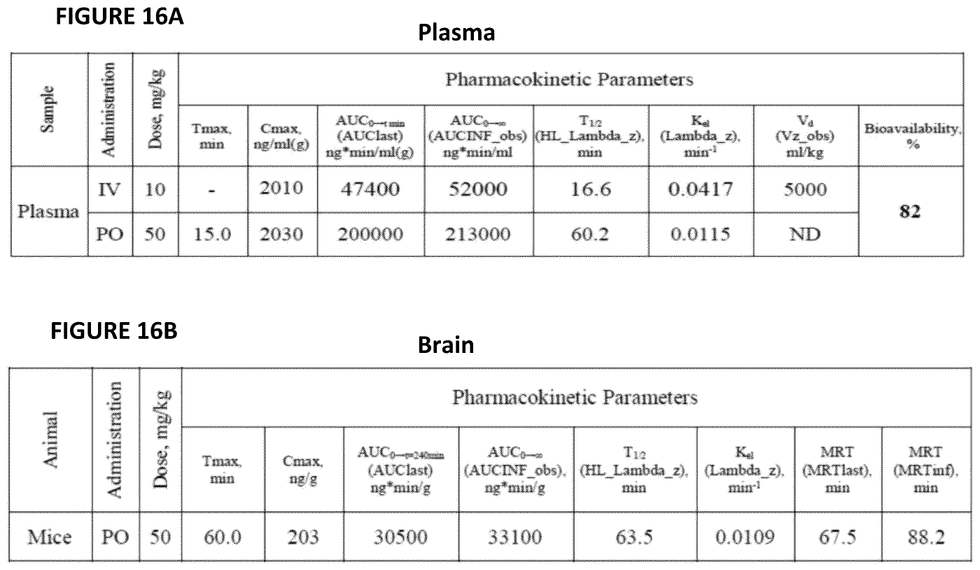

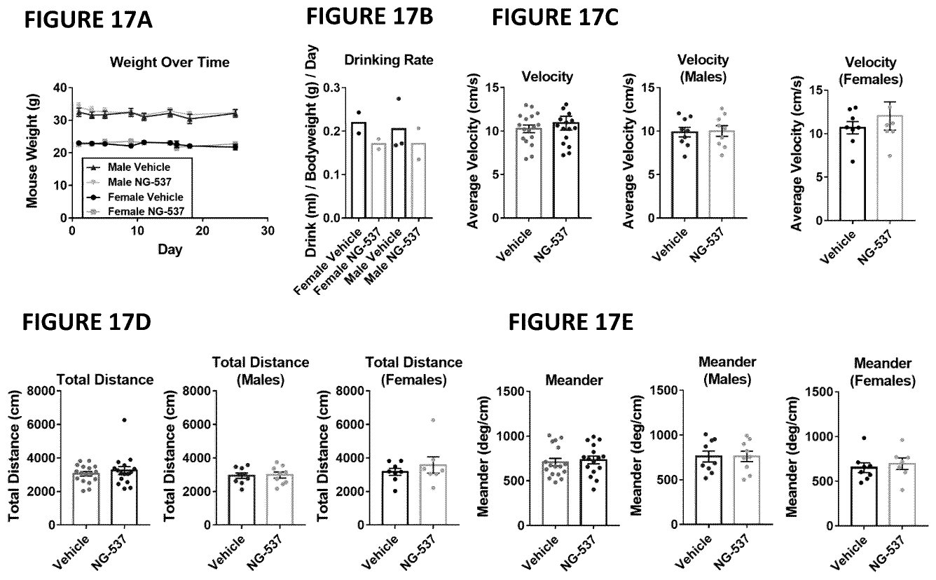

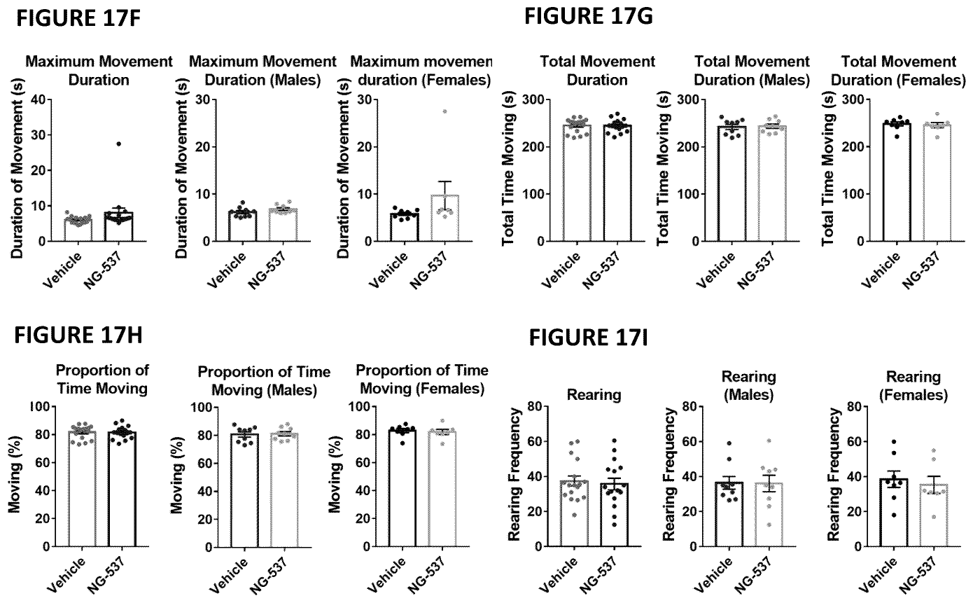

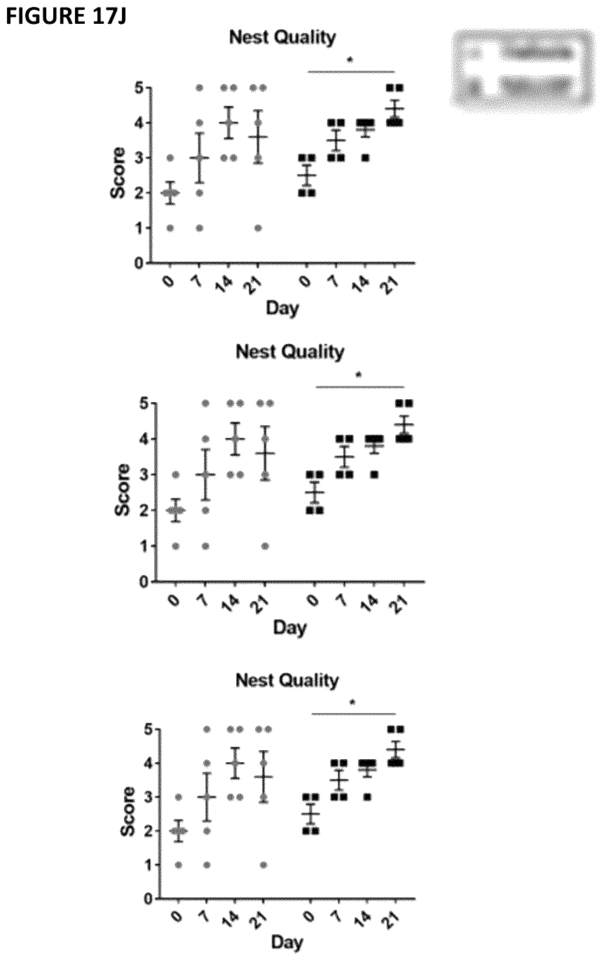

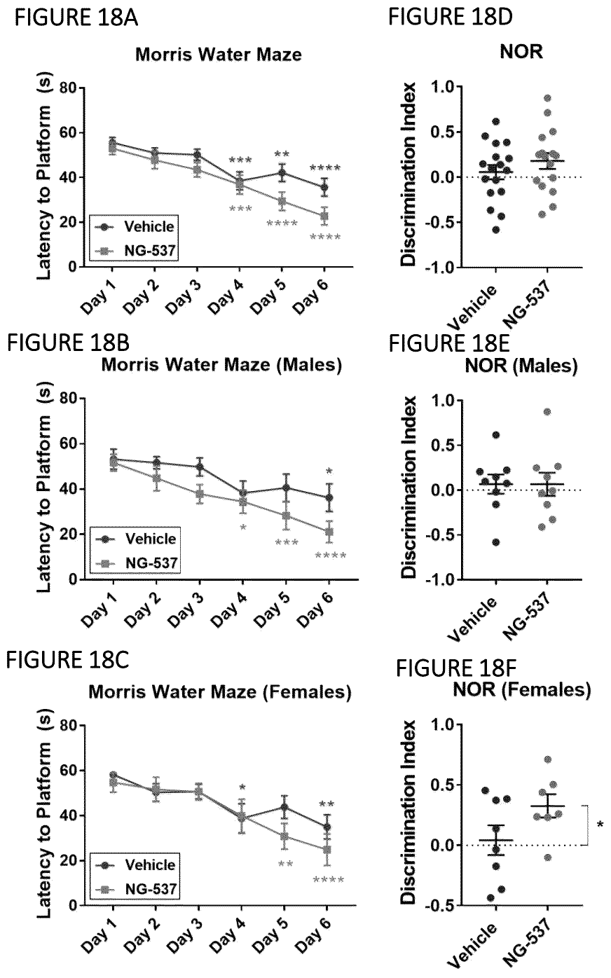

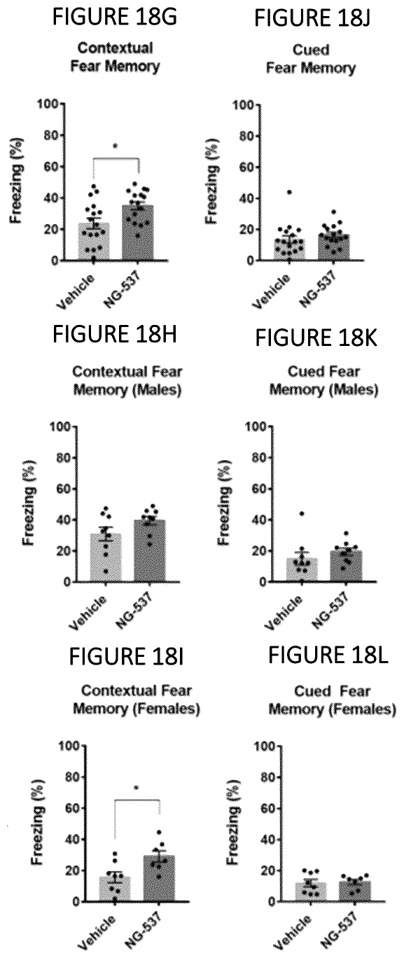

The inventors next aimed to test the effect of QR2 inhibition in AD model mice using YB-537, taking advantage of its extremely high specificity, solubility and lack of toxicity. First, the inventors determined YB-537 pharmacokinetics (PK) and oral bioavailability and assessed any acute observable toxicity in mice. It was found that YB-537 was well tolerated at 50 mg/kg p.o. or 10 mg/kg i.v. with no discernable adverse symptoms at any time point up to 24 h following administration. YB-537 was 82% bioavailable p.o., and peak concentrations of 203 ng/g (equivalent to ˜500 nM YB-537) were detected in the brain following ˜1 h when orally administered (FIG. 16). The inventors therefore opted to deliver YB-537 to AD model mice via their drinking water, so they may freely, and chronically, ingest the inhibitor at 50 mg/kg, with minimal intervention or trauma. The inventors chose to use 8-9 months old 5×FAD male and female mice in double-blind experiments, so that well progressed and strong symptoms and pathologies will be present in order to mimic clinically relevant cases in human patients. Animals were handled regularly for 2 weeks prior to the commencement of treatment and experimentation. Then, animals received the treatment for a week (beginning of experiment—week 1), and no adverse symptoms were seen or measured, and animals kept receiving the treatment throughout the month of experimentation. Other than an improvement in nest quality over time seen in animals receiving YB-537, no changes to general physical parameters or wellbeing were observed over the course of the month of treatment (FIG. 17). On the second week (week 2), behavioral experiments commenced, including Morris water maze (MWM) and novel object recognition (NOR), which were carried out in parallel, with one half of the animals (including both sexes and treatments) undergoing one of the paradigms in week 2, and the other in week 3. The inventors found that in MWM the escape latency of the group receiving YB-537 tended to be faster than the control group (FIG. 18A), though this was not statistically significant (RM two-way ANOVA, treatment: p=0.0694), and a similar pattern was seen both in male (FIG. 18B) and female (FIG. 18C) mice. Since learning in this paradigm was slow, and performance was poor even following 6 days of training (using 4 learning trials a day), a test day was not carried out and instead only the learning rate was measured. In the NOR task, mice receiving YB-537 tended to discern the novel object (FIG. 18D), though this was not statistically significant (one sample t test: p=0.0603), while no object discrimination was observed in any of the male mice groups (FIG. 18E). Contrastingly, female mice receiving YB-537 significantly preferred to investigate- and were able to discern the novel object, while females receiving vehicles did not (FIG. 18F). Finally, during week 4, all the mice underwent delay fear conditioning (DFC). Both control mice and those receiving YB-537 in drinking water showed similar baseline freezing in conditioning chambers, regardless of sex (both sexes combined—Vehicle 0.746±0.178%, n=17; YB-537 0.805±0.357%, n=16; Mann-Whitney test, p=0.3203; males—Vehicle 0.937±0.288%, n=9; YB-537 0.642±0.408, n=9; Mann-Whitney test, p=0.2073; females—Vehicle 0.531±0.188%, n=8; YB-537 1.016±0.657%, n=7; Mann-Whitney test, p=0.9747). Mice receiving YB-537 froze significantly more in response to the context (FIG. 18G), but not to the cue (FIG. 18J) compared to controls, while male mice did not significantly differ in response to context (FIG. 18H) or cue (FIG. 18K). However, female mice receiving YB-537 froze significantly more than controls in response to the conditioned context (FIG. 18I), but not to the cue (FIG. 18L). Overall, during a month-long experiment during which 8-9 month old 5×FAD mice of both sexes received 50 mg/kg of YB-537 in their drinking water, no adverse effects were seen and an improvement in cognitive function and nesting were measured.

### Example 9

Drinking YB-537 for 1 Month Significantly Reduces Brain Pathologies Associated with Dementia in 8-9 Months Old 5×FAD Mice

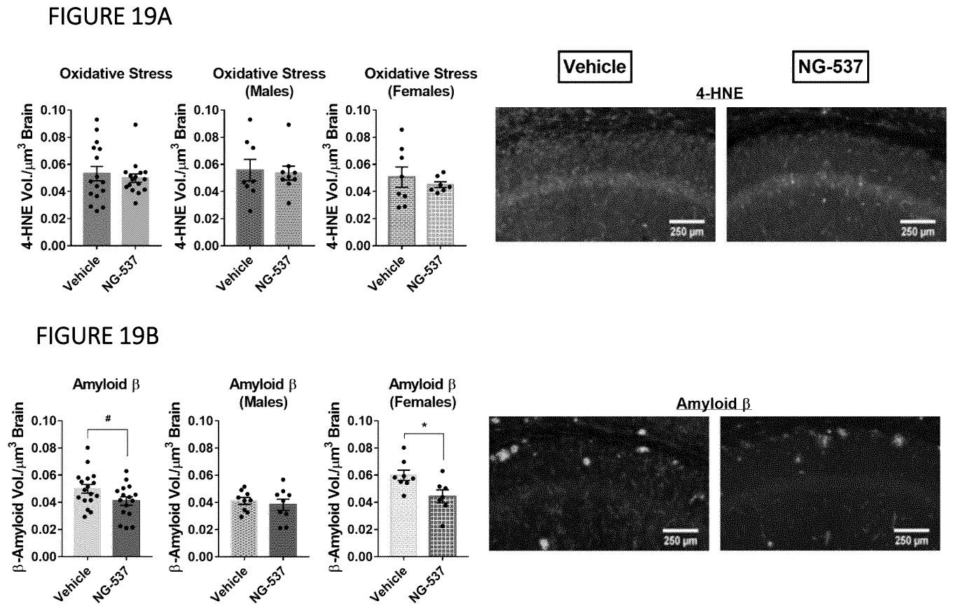

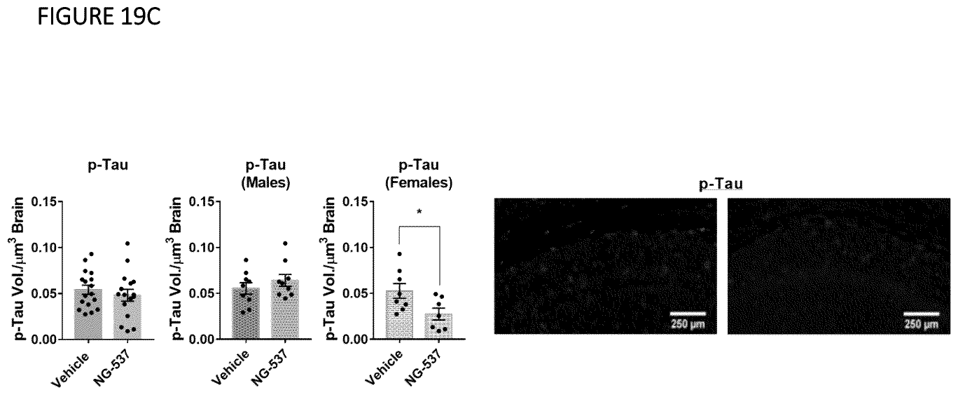

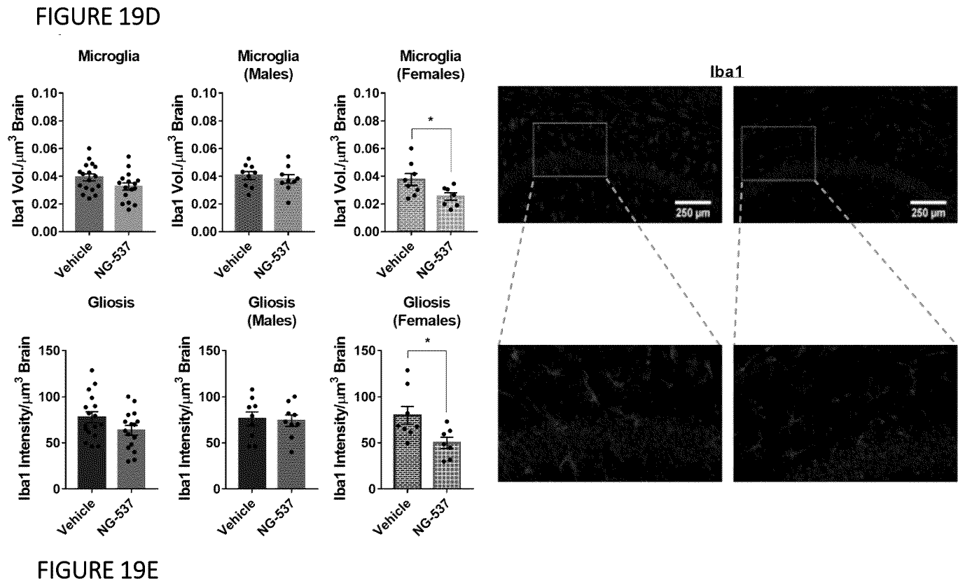

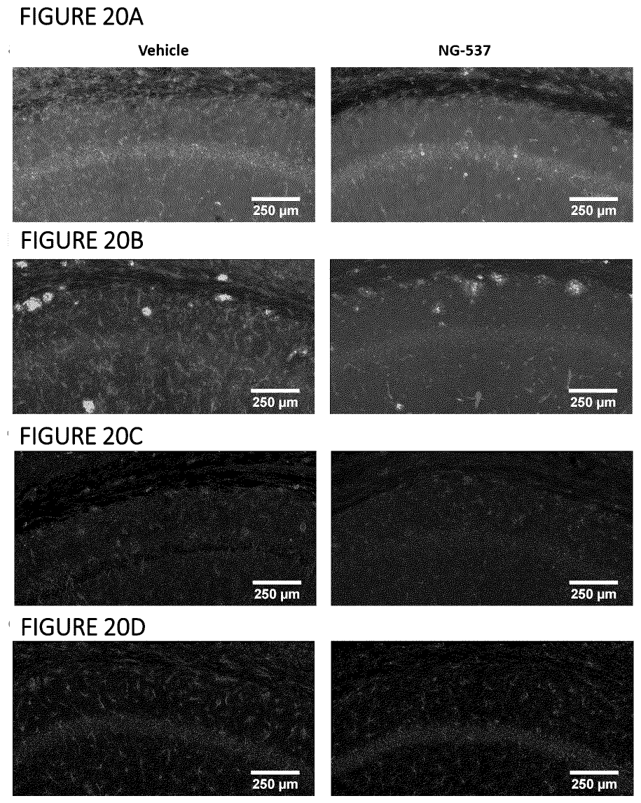

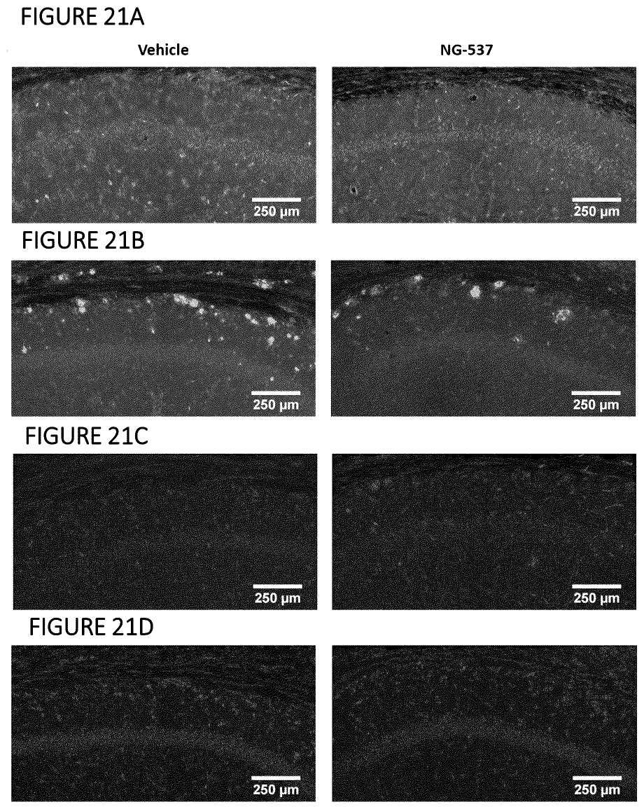

Following the completion of behavioral experiments, the inventors wished to assess brain pathologies associated with AD present in the 5×FAD mice used. Therefore, 5 days following the completion of the last behavioral experiment, and 1 month following the start of YB-537 consumption, all the mice were sacrificed and coronal sections of their fixed brains were made and used for immunohistochemistry. Since the CA1 region of the hippocampus is affected early on in the pathogenesis of AD, and this brain region is involved in the behavioral paradigms used here, the inventors acquired images of CA1. This was done with an Olympus IX83 confocal microscope using the same settings, antibodies and exposures (all slices and samples were prepared by the same experimenter and all images taken by the same experimenter) in all mice and a separate blind experimenter, unaware of the experimental conditions or details, measured and analyzed the signal detected using Imaris (Bitplane) software (see methods). For each mouse, three coronal sections were used per antibody. In each section, the same region of CA1 was acquired, using the same image frame size and resolution, at all possible depths of the section (Z-stack of the whole section), to allow measurement of the fluorescent antibody marker volume, which was normalized to the brain volume from which it was taken. First, the inventors measured oxidative stress, as indicated by lipid peroxidation product, 4-Hydroxynonenal (4-HNE, antibody: AbCam, ab48506). No difference in average 4-HNE volume was seen between mice receiving YB-537 or controls, though YB-537 prevented high- and low percentile measurements, altering distribution (F test, p=0.0516), which was much more tightly centered around the mean compared to controls, which showed greater variability (FIG. 19A, left histogram). Males showed no difference between groups (FIG. 19A, middle histogram). Females did not show any changes in mean 4-HNE volume, but have a significantly different distribution (F test, p=0.0052), with all animals receiving YB-537 showing measurements centered around the mean, while controls showed far greater variability (FIG. 19A, right histogram, and image panels). Next, amyloid R volume was measured (antibody: AbCam, ab201060), and a reduction was seen in mice receiving YB-537 compared to controls (FIG. 19B, left histogram), though no changes were seen in males (FIG. 19B, middle histogram). In contrast, female mice receiving YB-537 showed a significant reduction in amyloid β volume (FIG. 19B, right histogram, and image panels). When measuring p-tau volume (phosphorylated on Ser202, antibody: Thermo, MN1020), no changes were seen in the total population (FIG. 19C, left histogram) or males (FIG. 19C, middle histogram), but a significant reduction in p-tau volume was seen in females (FIG. 19C, right histogram, and image panels). Next, microglia volume was measured in CA1 (antibody: Iba1, AbCam, ab5076), and no change in the total (FIG. 19D, left histogram) or male (FIG. 19D, middle histogram) populations were seen, but a significant reduction in females receiving YB-537 was measured (FIG. 19D, right histogram, and image panels). In order to assess the activation state of the microglia measured in CA1 of the mice, the signal intensity of the microglia was measured (antibody: Iba1, AbCam, ab5076). Neither the total (FIG. 19E, left histogram) or male (FIG. 19E, middle histogram) populations showed any significant changes to microglia signal intensity, but females receiving YB-537 showed a significant reduction compared to controls (FIG. 19D, right histogram, and image panels). Taken together, 8-9 months old 5×FAD mice show a reduction in AD related brain pathologies and hallmarks following 1 month of YB-537 ingestions, with females showing far greater effect than males (FIGS. 20-21).

### DISCUSSION

Here, the inventors show that there is a functional consequence to the removal of such chronically high baseline levels of QR2, which results in a proteome that opposes that seen in AD brains, where high QR2 expression levels are found, and in which QR2 polymorphisms adversely affect pathology. The resulting, AD-antagonistic changes to the proteome include mRNA translation and mitochondrial respiration, cell-cell communication and various signaling pathways that confer a distinctly divergent cellular phenotype to that seen in AD, and strengthens the case for QR2 inhibition as a novel therapeutic avenue with which to treat age-related metabolic stress in the brain.

The disclosed herein QR2i showed promising results in double blind behavioral experiments, enhancing cortical memory in rats and hippocampal memory in mice. Furthermore, inhibiting QR2 chronically in the hippocampus of mice led to an increase in CD73, similarly to that found in QR2Δ HCT116 cells in which QR2 was genetically removed, both validating the QR2i in vivo and pointing to consistent outcomes across models.

Critically, water-soluble YB-537 is 82% bioavailable by p.o. administration, and is able to enter the mouse brain at relevant concentrations with a clearance half-life of ˜1 h upon acute oral dosing, and is non-toxic and well tolerated, which enables long-term oral dosing studies.

Taking advantage of this, the inventors found that by providing YB-537 in the drinking water of aged 5×FAD mice and thus chronically inhibiting QR2 for 1 month, a significant reduction in brain pathologies and improvement in memory was observed in these mice, in a double-blind experiment. Both male and female mice showed quicker learning in the MWM, while females demonstrated further significant improvement in NOR and DFC paradigms, compared to controls. Both p-tau and amyloid β were significantly diminished following QR2i in female mice, and the number- and activation of—microglia was also significantly reduced. Furthermore, an interesting outcome of prolonged QR2i in the aged AD mice, both in males and females, was the reduction in variance of oxidative stress in the brain. QR2i in the AD mice reduced extreme high- and low measures of oxidative stress product 4-HNE, suggesting a homogenization of redox state in the QR2i brains that was not possible with intact QR2 activity, in the context of the AD model rodent brain. New ways to help correct age-related deficits in the brains ability to maintain metabolic homeostasis, while acting as cognitive enhancers, are of key importance. Here, the inventors show that QR2i answer both demands, and cause no adverse side effects.

With the recent description of the QR2 pathway in the brain and its association with cognitive dysfunction in age, as well as with other metabolic and oxidative stress related pathologies, there is an exceptional opportunity to tackle neurodegenerative diseases such as AD via the subtle manipulation of QR2 mediated stress.

### Example 10

### Therapeutic Effects of QR2 Inhibition

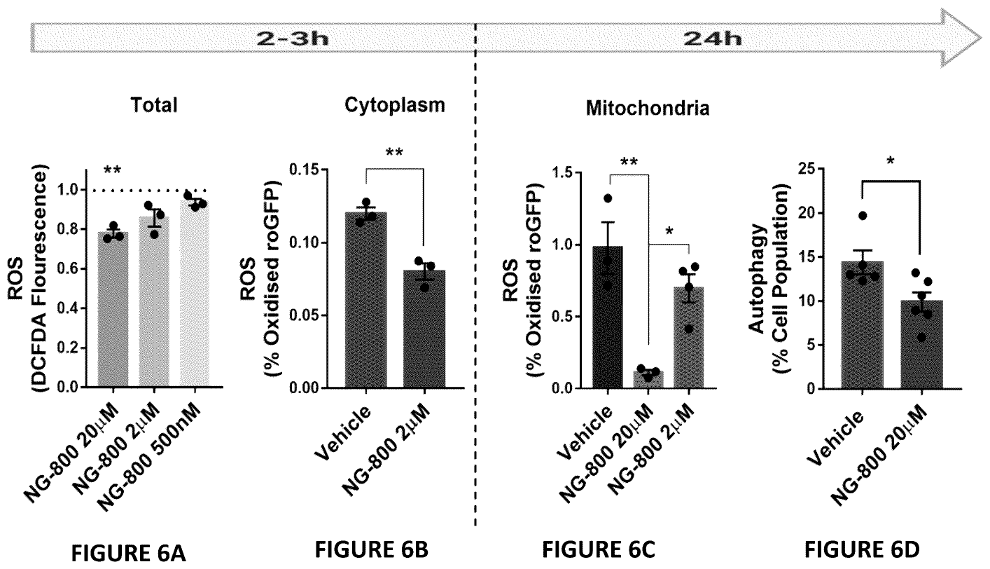

QR2 inhibition was also shown to be effective in reducing amount or levels of reactive oxygen species (ROS) and oxidative stress mediated autophagy (FIG. 6).

Further, QR2 inhibition was found to be effective in reducing inflammation (FIG. 7).

### Example 11

### Characterization and Optimization of QR2 Small Molecule Inhibitors

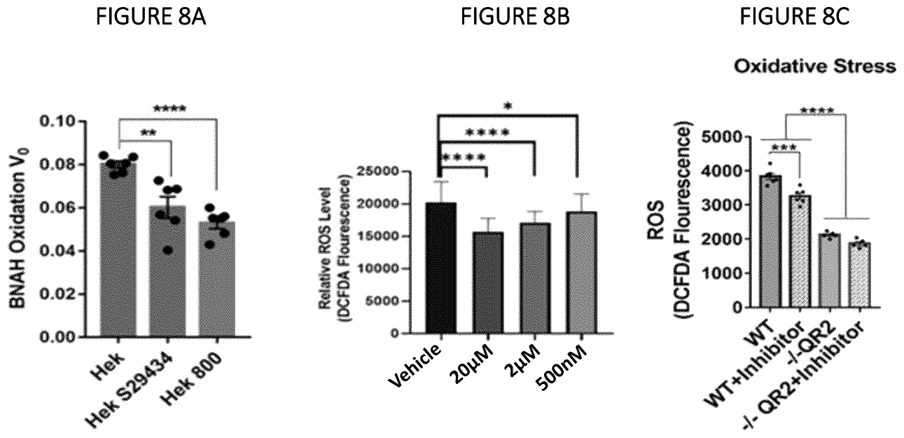

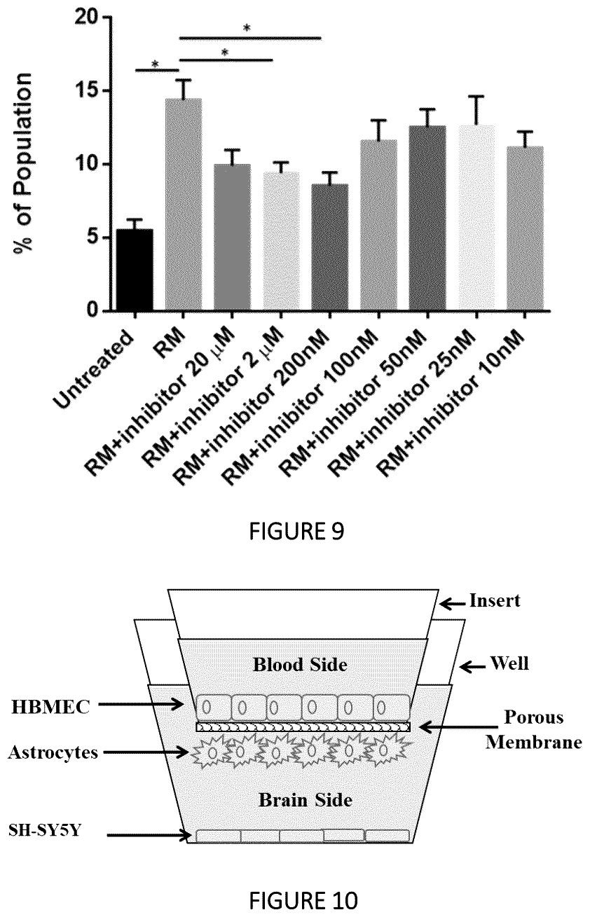

Preliminary results indicate that treatment of HEK293 cells, which endogenously express high levels of QR2, with QR2 inhibitors results in reduced activity of QR2 and reduced ROS levels in a dose-dependent manner (FIG. 8). Given that QR2 inhibition was shown to reduce autophagy in meiotic stress, the inventors also analyzed autophagy rates following the application of the inhibitors in HEK293 cells treated with rapamycin. The results indicated autophagy inhibition in QR2 attenuated cells (FIG. 9). Similar results were obtained using additional cells lines such as HCT 116 and VeroE6 cell lines (data not shown).

### Example 12

Blocking Cytokine-Induced Neuronal Damage with QR2 Inhibitors

COVID-19 infection leads to a robust inflammatory oxidative response that culminates in excessive production of immune-stimulating cytokines; a phenomenon also termed ‘cytokine storm’. Accordingly, the inventors test the responses of brain cells (primary neurons and microglia) to physiologically relevant stimuli, such as cytokines. Specifically, the inventors generate primary neuronal and microglia cultures as well as co-cultures, which are treated with pro-inflammatory cytokines such as IFNγ, TNFα, IL-6, and IL-10. The inventors treat primary cultures at two-time points (3 h and 24 h) and doses/combinations of cytokines and then, based on the above findings, and examine the following outcomes: cytotoxicity: (1) cell viability, tested using the Cell Proliferation Kit (XTT) (biological industries, Israel); (2) cell morphology, examine morphology filamentous (F) actin being visualized by Acti-stain 488 phalloidin; (3) ROS levels are measured as described above, by DCFDA/roGFP; (4) autophagy, as described above, and (5) Lysosomal pH, as follows. The LysoSensor™ pH Indicator (ThermoFisher) is used. This probe can be used singly (or potentially in combination with other markers, e.g., autophagy) to investigate the acidification of lysosomes and alterations of lysosomal function or trafficking that occur in cells. Published data indicate that compounds such as CQ and hydroxychloroquine (HCQ) affect lysosomal acidity, thus are used as controls. The inventors also use Tamoxifen and BafA1, which were shown to induce lysosomal pH alkalization, as additional controls.

### Example 13

### Synergism of QR2 Inhibitors and Known Anti-Inflammatory Drugs

The inventors test the synergy between dexamethasone and QR2 inhibition in primary brain cells (neurons and glia). As described above, the inventors study the blockage of the altered phenotype/s in cytokine-treated cultures. Further, the inventors examine the synergy between protein kinase R (PKR) inhibitors and QR2 inhibitor, in primary brain cells as described above.

### Example 14

### In-Vivo Delivery of QR2-Specific Small Molecules Inhibitors (QR2i)

The inventors establish a pharmacokinetic/pharmacodynamic profile and assess the ability of QR2 inhibiting compounds to cross the blood brain barrier (BBB) in mice after oral administration. SARS-CoV-2-mediated brain pathology is studied in an ACE2 mouse model. To assess pharmacokinetics and BBB permeability, mice receive the inhibitors as mentioned above, and distribution in blood and brain is measured over several time points. The identification of the compounds given is performed by Liquid chromatography-Mass spectrometry (LC-MS).

### Example 15

### The Effect of SARS-CoV-2 on the Integrity of Human BBB (In Vitro)

Human Brain Microvascular Cells (HBMEC), Human neuronal cells (SH-SY5Y), and human astrocytes (HA) are purchased from ATCC to develop in vitro trans-well BBB model explained in FIG. 10. Human coronavirus 229E (ATCC® VR-740™) is also purchased from ATCC and maintained in the laboratory condition in human lung fibroblast cells MRC-5 (ATCC® CCL-171™). The viral dose of infection is determined based on the viral nucleocapsid protein through Nucleocapsid (N) Protein ELISA Kit (RayBiotech, Inc. Cat #ELV-COVID19N). The cell infected with a multiplicity of infection (MOI) >5, is washed after adsorption, and subsequently harvested 24 hours post-infection for viral titer, gene, and protein estimation. To quantify the integrity of the endothelial monolayer, TEER values across monolayers of endothelial cultures is measured with an Endohm-12 electrode chamber connected to an EVOMX Epithelial Voltohmmeter (World Precision Instruments, Sarasota, FL, USA) once per day for up to three days after exposure. Following exposure to the cells, a detailed cytotoxicity study is conducted at The University of Texas Rio Grande Valley (UTRGV), USA.

Since it has been found that SARS-CoV-2 can infect human brain cells through ACE2 receptors, finding the level of infection in BBB is important to observe. Therefore, HBMEC, HA, and SH-SY5Y cells are exposed to SARS-CoV-2 for 1 h and washed, to remove all unbound viruses. Following that, these cells are incubated for 24, 48, and 72 h post-infection. After the incubation, HBMEC, HA, and SH-SY5Y cells are harvested, and the supernatant is used to measure the Nucleocapsid protein (N-protein) production using the SARS-CoV-2N Protein ELISA Kit (RayBiotech, Inc.). The time of SARS-CoV-2 infection is extended based on the N-protein level using enzyme linked immunosorbent assay (ELISA). The N-protein expression is used to cross-validate the gene expression through PCR of the infected cells as per published protocol 2019-nCoV RUO Kit (Integrated DNA Technologies, #10006713).

Cytotoxicity of the SARS-CoV-2 is determined by using the alamarBlue™ assay (Invitrogen) and 3-(4,5-dimethylthiazol-2-yl)-2,5-diphenyltetrazolium bromide MTT assay (SIGMA). In vitro, the BBB model is exposed to SARS-CoV-2 for 24, 48, and 72 h. After treatment, cells are incubated with MTS reagent in 100 μl cell media. After incubation for 1 h at 37° C., the absorbance at 490 nm is measured with a BioTek plate reader (BioTek, USA). Cells without treatment are considered as blank and control is used as a 100% viability value. The cell viability is calculated as sample/control×100%. Based on the MTS assay, an optimized concentration of virus and exposure time is selected for further studies.

Some evidence has suggested that SARS-CoV-2 can infect brain cells and affect the integrity of BBB. In this regard, the expression and localization of zonula occludens protein (ZO-1) at the BBB is critical for characterizing the integrity of BBB. The ZO-1 protein expression is observed after 48 and 72 h of post-exposure to the virus with rabbit anti-ZO-1 polyclonal antibody (Life Technologies, Carlsbad, CA, USA; #61-7300) and donkey anti-rabbit Alexa Fluor 488 secondary antibody (Life Technologies). Cultures are also incubated with 4′,6-diamidino-2-phenylindole (DAPI; Life Technologies) to detect cell nuclei to determine cell counts for each sample. Endothelial monolayers are imaged using an Olympus IX81 (Olympus America, Center Valley, PA, USA) fluorescence microscope and analyzed using MetaMorph software (Molecular Devices, Sunnyvale, CA, USA).

It is important to observe the effect of SARS-CoV-2 exposure on neuroinflammation in BBB and neuronal cells. Since ROS is a direct indicator of neuroinflammation, this observation establishes the direct correlation between SARS-CoV-2 infection and neuroinflammation. Therefore, the inventors measure ROS produced from HBMEC, SH-SY5Y, HA cells following exposure to the virus for 72 h. The ROS assay is erformed using dichlorofluorescein diacetate assay (DCF-DA; Molecular Probes, Eugene, OR, USA). At the end of the experiment, fluorescence is visualized (485 excitations and at 528 emission spectra) in an Olympus IX51 microscope (Olympus America Inc., Center Valley, PA, USA) and the result are expressed as mean relative fluorescence units (RFU) with respect to different treatments. Further characterization is done concerning the cyto/chemokine profile of the cell after QR2 as per the manufacturer's protocol (RayBiotech, GA). The QR2 gene expression is measured by PCR.

Considering the importance of the QR2 inhibitors in reducing neuroinflammation, the inventors perform autophagosome-based small molecule retention and release in the human brain and neuronal cells through the IL-1b ELISA kit (eBiosciences). The changes in IL-1b level in the supernatant indicate the autophagy-based small molecule retention in the cells. In this regard, an optimized concentration of QR2 is introduced to this in vitro model to observe the effect of these molecules on reducing neuroinflammation and normalization of ROS.

### Example 16

### The Effect of QR2 Inhibition on the Neuropathology Induced by SARS-CoV-2 in a Mouse Model

SARS-CoV-2 is maintained in MRC-5 as mentioned in the previous section. The virus is intraperitoneally (i.p) infected with 4×10⁵ PFU of SARS-CoV-2. Three groups of B6.Cg-Tg(K18-ACE2)2Prlmn/J transgenic mice are used. Mock-infected mice receive a similar volume of phosphate-buffered saline (PBS). Mice are sacrificed by CO₂ anoxia 72 h post-infection (p.i.). The brain and liver are collected and frozen at −80° C. for further analyses.

Gene expression and qRT-PCR—Total RNA from frozen brain samples is extracted using TRIzol reagent (Invitrogen), and residual genomic DNA is removed with the Turbo DNA-free kit (Ambion, Austin, TX). RNA is extracted using a NucleoSpin RNA II kit (Macherey-Nagel, Bethlehem, PA) according to the manufacturer's instructions. Real-time PCR amplification is carried out using the HotStart-IT SYBR green qPCR master mix (USB Corporation, Cleveland, OH) on an ABI 7300 system (Applied Biosystems, Foster City, CA). The gene expression that is characterized includes inflammatory markers IL-6, TNFα, MCP-1, and COVID-N protein using publicly disclosed primers and protocol. Gene expression is normalized to the expression of the GAPDH gene as endogenous control and expressed as a ratio of gene expression in PBS-treated mice.

ELISA of SARS-CoV-2 induced neuroinflammatory factors—Determination of the levels of IFN-β (PBL, Piscataway, NJ), IL-6, tumor necrosis factor-alpha (TNF-α) (BD, Canada), CXC chemokine ligand 10 (CXCL10), CC chemokine ligand 2 (CCL2) (eBiosciences, CA), and CXCL1 (R&D Systems, MN) in blood plasma samples is carried out by assay kit according to the manufacturer's instructions (Ray Biotech, GA).

Viral titration in the brain tissue of SARS-CoV-2 infected mice—Viral particles are measured on brain lysates from infected mice in 96-well plates. Cytopathic effects, characterized by syncytia and cell lysis, are recorded at 72 h post-infection, and virus titers (TCID50) are determined according to the Reed-Muench method.

In vivo assessment of BBB integritySARS-CoV-2 infected mice. Changes in blood-brain barrier permeability are assessed using sodium fluorescein (NaF) (Sigma-Aldrich). Briefly, mice are infected for 72 h and intraperitoneally (i.p) injected with 100 μl of 10% NaF in PBS for 1 h before euthanasia. Cardiac blood are collected, transcardial perfusion with PBS is performed, and brains are removed, weighed, and homogenized in PBS (1:10 [w/v]). NaF content is measured on a Synergy 4 microplate fluorometer (Biotek, VT) with excitation at 485 nm and emission at 530 nm using standards ranging from 0.78 μg/ml to 5 g/ml. The NaF concentration in the brain is normalized to serum NaF concentrations for each mouse to allow comparisons among mice and is calculated as follows: (microgram of NaF in the brain/milligram of the brain)/(microgram of NaF in sera/microliter of blood). Data is expressed as a fold increase in fluorescence compared to the levels in uninfected mice.

In vivo expression of ZO1 in SARS-CoV-2 infected mice BBB. SARS-CoV-2 infected mice brain are snap-frozen in Tissue-Tek OCT Compound (Sakura Finetex), for immunohistochemical analysis. The brain tissue slice is incubated with primary antibodies (ZO-1, occludin, and VE-cadherin antibodies diluted 1:100; all antibodies from LSBio, Seattle, WA) overnight at 4° C., then it is incubated with fluorescein isothiocyanate (FITC)-labeled goat anti-rabbit secondary antibody (1:500) (1 hr at room temperature), and mounted in antifade medium containing 4′,6′-diamidino-2-phenylindole (DAPI) counterstain. Fixed tissue is imaged with a Zeiss LSM700 static Observer Z1 confocal microscope (63×) (Zeiss, Canada) and analyzed using ZEN 2009 software. For brain immunolocalization of ZO-1, VE-cadherin, and occludin, mouse brain sections are fixed in paraformaldehyde and embedded in paraffin and the antigens are retrieved by incubating the sections with primary antibodies (1, 2.5, and 10 μg/ml, respectively; LSBio, WA) for 1 h in a Ventana automated machine (Ventana Medical Systems, AZ), and OmniMap anti-rabbit secondary antibodies conjugated to horseradish peroxidase (HRP) for 16 min. The sections are then counterstained with standard hematoxylin protocol.

Therapeutic efficacy of the QR2i in SARS-CoV-2 infection in K18-hACE2 transgenic mice. Briefly, once the infection is established, the animals are challenged with QR2i and viral replication is monitored in the infected blood serum at different time points after drug treatment. Mice are injected i.p. with SARS-CoV-2 (10⁴ TCID₅₀/mouse). Three days of the following infection, 5 experimental groups of mice (n=16/group) are set up, as follows: Group-1 serves as the negative control; Group-2 serves as infected and untreated control; Group-3 is infected and treated with one optimized dose of QR2i; Group-4 is infected and treated with an optimized dose of dexamethasone; and Group-5 is infected and treated with an optimized dose of QR2i and dexamethasone. The exact dosage of drugs and dosing frequency is determined by the in vivo study and toxicity profiles as described above. Animals are sacrificed on day 7 after drug treatment and the tissue and serum are collected with full-body perfusion. The levels of viral RNA copies/ml in the plasma are collected on days 0, 1, 3, 5, and 7 after QR2i administration and measured by automated Abbott RealTime SARS-CoV-2 Amplification Reagent Kit. The plasma sample obtained from these mice is also tested for liver enzyme levels and renal functions as mentioned. In this regard, mice brain tissue is used to observe the ROS and neuroinflammation-related markers.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1.-12. (canceled)

13. A method for preventing or treating a QR2-related disease or disorder in a subject in need thereof, the method comprising administering to the subject a therapeutically effective amount of a pharmaceutical composition comprising the compound or a pharmaceutically acceptable salt thereof, wherein the compound is represented by or comprising Formula 1: 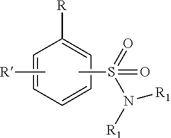
wherein: [symbol]
represents a single or a double bond;
R′ is any one of: 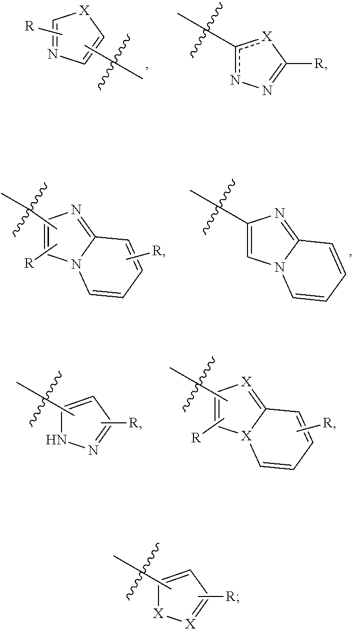
each R independently represents hydrogen, or a substituent comprising halogen, —NO₂, —CN, —OH, —CONH₂, —CONR″₂, —CNNR″₂, —CSNR″₂, —CONH—OH, —CONH—NH₂, —NHCOR″, —NHCSR″, —NHCNR″, —NC(═O)OR″, —NC(═O)NR″, —NC(═S)OR″, —NC(═S)NR″, —SO₂R″, —SOR″, —SR″, —SO₂OR″, —SO₂N(R″)₂, —NHNR″₂, —NNR″, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —C(═O)N(C₁-C₆ alkyl)₂, —CO₂H, —CO₂R″, —OCOR″, —OCOR″, —OC(═O)OR″, —OC(═O)NR″, —OC(═S)OR″, —OC(═S)NR″, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, wherein R″ represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof;
each X independently comprises C, CH, S, O, N or NH as allowed by valency;
each R₁ independently represents H, or a substituent comprising optionally substituted C₁-C₆ alkyl, hydroxy(C₁-C₆ alkyl), C₁-C₆ haloalkyl, optionally substituted C₃-C₈ cycloalkyl, optionally substituted C₁-C₆ alkyl-cycloalkyl, optionally substituted C₁-C₆ alkyl-aryl, optionally substituted C₁-C₆ alkyl-heteroaryl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or both R1 are interconnected, so as to form a cyclic ring, thereby preventing or treating a QR2-related disease or disorder in the subject.

14. The method of claim 13, wherein said QR2-related disease or disorder is selected from the group consisting of: a neurodegenerative disease or disorder, a cell-proliferative disease or disorder, malaria, drug direct or indirect toxicity, NAD-related metabolic toxicity, epilepsy, ischemia, depression and/or anxiety, restenosis, glaucoma, immune-response related disease, and any combination thereof.

15. The method of claim 14, wherein said neurodegenerative disease or disorder is selected from the group consisting of: Alzheimer's disease, dementia, Parkinson's disease, Huntington's disease, Down syndrome, amyotrophic lateral sclerosis (ALS), prion disease, and any combination thereof.

16. The method of 13, wherein said preventing or treating comprises inhibiting QR2 function or activity in a cell of said subject.

17. The method of claim 16, wherein said cell is a nerve cell, a glia cell, or both, of said subject.

18. The method of 13, wherein said administering comprises: oral administration, topical administration, nasal administration, sublingual administration, buccal administration, a systemic administration, or any combination thereof.

19. The method of 13, further comprising diagnosing said QR2-related disease or disorder in said subject.

20. The method of claim 19, wherein a subject diagnosed with QR2-related disease or disorder is characterized by increased QR2 function or activity compared to a control subject.

21. The method of claim 19, wherein said diagnosing comprises determining the function or activity of QR2 in said subject or in a sample derived therefrom.

22. The method of 13, wherein said preventing or treating comprises: reducing the amount or level of reactive oxygen species (ROS), modulating autophagy, reducing or inhibiting inflammation, or any combination thereof, in said subject.

23. The method of claim 22, wherein said modulating autophagy comprises increasing autophagy or reducing autophagy.

24. A compound represented by any one of Formulae: 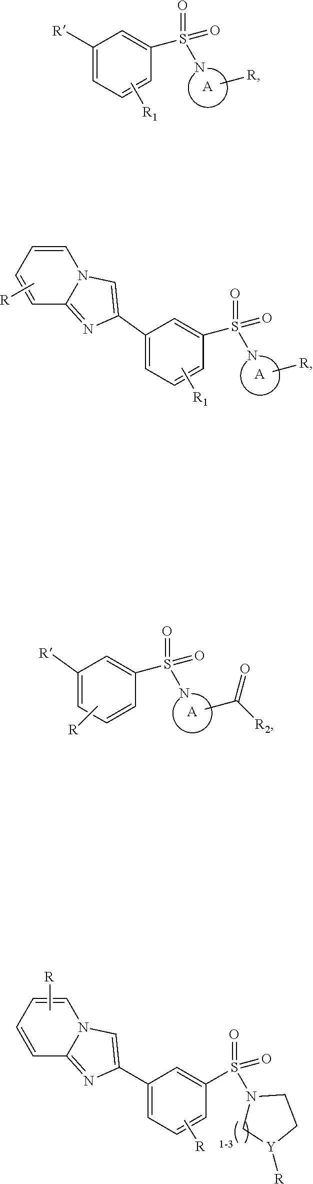 [symbol]
represents a single or a double bond;
R′ is any one of: 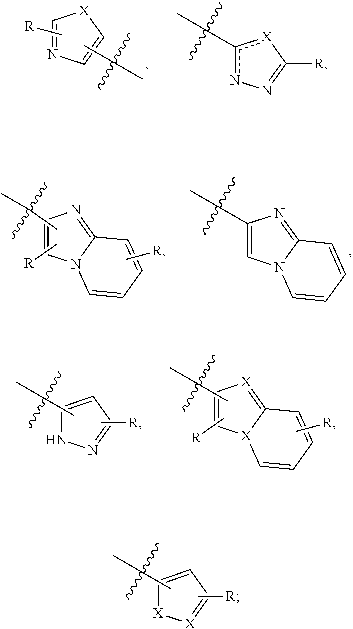
each R independently represents hydrogen, or a substituent comprising halogen, —NO₂, —CN, —OH, —CONH₂, —CONR″₂, —CNNR″₂, —CSNR″₂, —CONH—OH, —CONH—NH₂, —NHCOR″, —NHCSR″, —NHCNR″, —NC(═O)OR″, —NC(═O)NR″, —NC(═S)OR″, —NC(═S)NR″, —SO₂R″, —SOR″, —SR″, —SO₂OR″, —SO₂N(R″)₂, —NHNR″₂, —NNR″, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —C(═O)N(C₁-C₆ alkyl)₂, —CO₂H, —CO₂R″, —OCOR″, —OCOR″, —OC(═O)OR″, —OC(═O)NR″, —OC(═S)OR″, —OC(═S)NR″, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof;
R″ represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof;
each X independently comprises C, CH, S, O, N or NH as allowed by valency;
Y comprises N or NH as allowed by valency;
each R1 independently represents a substituent comprising optionally substituted C₁-C₆ alkyl, hydroxy(C₁-C₆ alkyl), C₁-C₆ haloalkyl, optionally substituted C₃-C₈ cycloalkyl, optionally substituted C₁-C₆ alkyl-cycloalkyl, optionally substituted C₁-C₆ alkyl-aryl, optionally substituted C₁-C₆ alkyl-heteroaryl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or both R1 are interconnected, so as to form a cyclic ring;
A comprises optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof.

25. The compound of claim 24, wherein R′ is

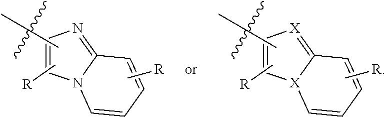

26. The compound of claim 24, wherein the compound is represented by Formula: [chemical structure]
